(19) United States
(12) Patent Application Publication
(10) Pub. No.: US 2026/0317382 A1
(43) Pub. Date: Oct. 15, 2026

(54) METHOD FOR DESIGNING RNA-BINDING PROTEIN UTILIZING PPR MOTIF, AND USE THEREOF

(71) Applicant: KYUSHU UNIVERSITY, NATIONAL UNIVERSITY CORPORATION, Fukuoka-shi (JP)

(72) Inventors: Takahiro Nakamura, Fukuoka-shi (JP); Yusuke Yagi, Fukuoka-shi (JP); Keiko Kobayashi, Fukuoka-shi (JP)

(73) Assignee: KKYUSHU UNIVERSITY, NATIONAL UNIVERSITY CORPORATION, Fukuoka-shi (JP)

(21) Appl. No.: 19/653,752

(22) Filed: Apr. 21, 2026

(30) Foreign Application Priority Data
Oct. 21, 2011 (JP) 2011-231346

(51) Int. Cl. C07K 14/415; C12N 15/10

(52) U.S. Cl. CPC C07K 14/415; C12N 15/102

(57) ABSTRACT

A method for designing a protein capable of binding in an RNA base selective manner or RNA base sequence specific manner is provided. The protein of the present invention is a protein containing one or more of PPR motifs (preferably 2 to 14 PPR motifs) each consisting of a polypeptide of 30- to 38-amino acid length represented by the formula 1 (wherein Helix A is a moiety of 12-amino acid length capable of forming an α-helix structure, and is represented by the formula 2, wherein, in the formula 2, A₁ to A₁₂ independently represent an amino acid; X does not exist, or is a moiety of 1- to 9-amino acid length; Helix B is a moiety of 11- to 13-amino acid length capable of forming an α-helix structure; and L is a moiety of 2- to 7-amino acid length represented by the formula 3, wherein, in the formula 3, the amino acids are numbered “i” (−1), “ii” (−2), and so on from the C-terminus side, provided that Lᵢᵢᵢ to Lᵥᵢᵢ may not exist), and combination of three amino acids A₁, A₄ and Lᵢᵢ, or combination of two amino acids A₄, and Lᵢᵢ is a combination corresponding to a target RNA base or base sequence.

### CROSS-REFERENCE TO RELATED APPLICATION

This application is a Divisional of U.S. patent application Ser. No. 18/221,440, filed on Jul. 13, 2023, which is a Divisional of U.S. patent application Ser. No. 17/195,449, filed on Mar. 8, 2021 (issued as U.S. Pat. No. 11,742,056), which is a Divisional of U.S. patent application Ser. No. 16/894,295, filed on Jun. 5, 2020 (issued as U.S. Pat. No. 10,943,671), which is a Divisional of U.S. patent application Ser. No. 16/431,429, filed on Jun. 4, 2019 (issued as U.S. Pat. No. 10,679,731), which is a Divisional of U.S. patent application Ser. No. 15/962,127, filed on Apr. 25, 2018 (issued as U.S. Pat. No. 10,340,028), which is a Divisional of U.S. patent application Ser. No. 15/335,243, filed on Oct. 26, 2016 (issued as U.S. Pat. No. 9,984,202), which is a Divisional of U.S. patent application Ser. No. 14/352,697, filed on Jul. 22, 2014 (issued as U.S. Pat. No. 9,513,283), which is a U.S. National Stage entry of International Application No. PCT/JP2012/077274, filed on Oct. 22, 2012, which claims priority to Japanese Patent Application No. 2011-231346, filed on Oct. 21, 2011. The entirety of each of the foregoing is incorporated herein by reference.

### SEQUENCE LISTING

The sequence listing that is contained in the file named is “P24436US07_Sequence_Listing.xml” having a size of 570,786 bytes and created on May 13, 2026, is incorporated herein by reference in its entirety.

### TECHNICAL FIELD

The present invention relates to a protein capable of selectively or specifically binding to an intended RNA base or RNA sequence. According to the present invention, a pentatricopeptide repeat (PPR) motif is used. The present invention can be used for identification and design of an RNA binding protein, identification of a target RNA of a PPR protein, as well as functional control of RNA. The present invention is useful in the medical field, agricultural field, and so forth.

### BACKGROUND ART

In recent years, techniques of binding nucleic acid binding protein factors elucidated by various analyses to an intended sequence have been established and utilized. By using such a sequence-specific binding, it is becoming possible to analyze intracellular localization of a target nucleic acid (DNA or RNA), eliminate a target DNA sequence, or control (activate or inactivate) expression of a gene coding for a protein existing downstream from such a target sequence.

Although there are being conducted research and development utilizing zinc finger proteins (Non-patent document 1) and TAL effectors (Non-patent document 2, Patent document 1), which are protein factors that act on DNA, as protein engineering materials, development of protein factors that specifically act on RNA is still extremely limited. This is because any general correspondence between affinity to RNA of amino acid sequences constituting proteins and bindable RNA sequences has been scarcely elucidated, or there is no such correspondence. Concerning the pumilio protein constituted by repetition of two or more puf motifs each consisting of 38 amino acids, it has been exceptionally demonstrated that one puf motif binds to one RNA base (Non-patent document 3), and it is being attempted to develop a novel protein having an RNA binding property and a technique of modifying RNA binding property by using the pumilio proteins (Non-patent document 4). However, the puf motifs are highly conserved, and exist only in an extremely small number. Therefore, they are used only for creation of a protein factor that interacts with a limited RNA sequence.

The PPR proteins (proteins having the pentatricopeptide repeat (PPR) motif) have been identified on the basis of genome sequence information (Non-patent document 5), which proteins constitute such a large family consisting of about 500 members only for plants. Although the PPR proteins are nuclear-encoded, they chiefly act for control of organelles (chloroplasts and mitochondria) at the RNA level, cleavage, translation, splicing, editing, and stability of RNA in a gene-specific manner. The PPR proteins typically have a structure comprising about 10 contiguous poorly conserved 35-amino acid motifs, i.e., PPR motifs, and it is considered that the combination of the PPR motifs is responsible for the sequence-selective binding with RNA. Almost all the PPR proteins consist of only the repeats of about 10 PPR motifs, and in many cases, any domain required for expression of catalytic action cannot be found in them. Therefore, it is considered that the identity of the PPR proteins is an RNA adapter (Non-patent document 6).

The inventors of the present invention proposed a method for modifying an RNA-binding protein using this PPR motif (Patent document 2).

### PRIOR ART REFERENCES

### Patent Documents

Patent document 1: WO2011/072246
Patent document 2: WO2011/111829

### Non-Patent Documents

Non-patent document 1: Maeder, M. L., Thibodeau-Beganny, S., Osiak, A., Wright, D. A., Anthony, R. M., Eichtinger, M., Jiang, T., Foley, J. E., Winfrey, R. J., Townsend, J. A., et al. (2008), Rapid “open-source” engineering of customized zinc-finger nucleases for highly efficient gene modification, Mol. Cell, 31, 294-301
Non-patent document 2: Miller, J. C., Tan, S., Qiao, G., Barlow, K. A., Wang, J., Xia, D. F., Meng, X., Paschon, D. E., Leung, E., Hinkley, S. J., et al. (2011), A TALE nuclease architecture for efficient genome editing, Nature Biotech., 29, 143-148.
Non-patent document 3: Wang, X., McLachlan, J., Zamore, P. D., and Hall, T. M. (2002), Modular recognition of RNA by a human pumilio-homology domain, Cell, 110, 501-512
Non-patent document 4: Cheong, C. G., and Hall, T. M. (2006), Engineering RNA sequence specificity of Pumilio repeats, Proc. Natl. Acad. Sci. USA, 103, 13635-13639
Non-patent document 5: Small, I. D., and Peeters, N. (2000), The PPR motif-a TPR-related motif prevalent in plant organellar proteins, Trends Biochem. Sci., 25, 46-47
Non-patent document 6: Woodson, J. D., and Chory, J. (2008), Coordination of gene expression between organellar and nuclear genomes, Nature Rev. Genet., 9, 383-395

### SUMMARY OF THE INVENTION

### Object to be Achieved by the Invention

The properties of the PPR proteins as an RNA adapter are expected to be determined by the properties of the PPR motifs constituting the PPR proteins and combination of a plurality of the PPR motifs. However, correlation of the amino acid constitution and function thereof are scarcely clarified. If amino acids that function when the PPR motifs exhibit the RNA-binding property are identified, and relation between structure of a PPR motif and a target base is elucidated, a protein capable of binging to an RNA having arbitrary sequence and length may be constructed by artificially manipulating structure of a PPR motif or combination of a plurality of PPR motifs.

### Means for Achieving the Object

In order to achieve the aforementioned object, the inventors of the present invention examined genetically analyzed PPR proteins, especially such PPR proteins involved in the RNA editing (modification of genetic information at the RNA level, especially conversion from cytosine (henceforth abbreviated as C) to uracil (henceforth abbreviated as U)), and target RNA sequences thereof, and elucidated that three amino acids in the PPR motifs (amino acids 1, 4, and “ii” (−2)) comprise information responsible for binding to a specific RNA base by using computational scientific techniques. More precisely, the inventors of the present invention found that the binding RNA base selectivity (also referred to as specificity) of the PPR motif is determined by three amino acids, i.e., the first and fourth amino acids contained in the first helix among two of the α-helix structures constituting the motif, as well as the second (“ii” (−2)) amino acid from the end (C-terminus side) in the moiety that can form a loop structure after the second helix, and accomplished the present invention.

The present invention thus provides the followings.

[1] A method for designing a protein that can bind to an RNA molecule in an RNA base-selective or RNA base sequence-specific manner, wherein:
the protein is a protein containing one or more of PPR motifs (preferably 2 to 14 PPR motifs) each consisting of a polypeptide of 30- to 38-amino acid length represented by the formula 1:

[F1]

(HelixA)-X-(HelixB)-L (Formula 1)

(wherein:
Helix A is a moiety of 12-amino acid length capable of forming an α-helix structure, and is represented by the formula 2:

[F2]

A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid; X does not exist, or is a moiety of 1- to 9-amino acid length;
Helix B is a moiety of 11- to 13-amino acid length capable of forming an α-helix structure; and L is a moiety of 2- to 7-amino acid length represented by the formula 3;

[F3]

Lᵥᵢᵢ-Lᵥᵢ-Lᵥ-Lᵢᵥ-Lᵢᵢᵢ-Lᵢᵢ-Lᵢ (Formula 3)
wherein, in the formula 3, the amino acids are numbered “i” (−1), “ii” (−2), and so on from the C-terminus side,
provided that Lᵢᵢᵢ to Lᵥᵢᵢ may not exist), and
combination of three amino acids A₁, A₄ and Lᵢᵢ, or combination of two amino acids A₄, and Lᵢᵢ is a combination corresponding to a target RNA base or base sequence.

[2] The method according to [1], wherein the combination of the three amino acids A₁, A₄ and Lᵢᵢ is a combination corresponding to the target RNA base or base sequence, and the combination of the amino acids is determined according to any one of the following propositions:
(3-1) when the three amino acids A₁, A₄, and Lᵢᵢ are valine, asparagine, and aspartic acid, respectively, the PPR motif can selectively bind to U (uracil);
(3-2) when the three amino acids A₁, A₄, and Lᵢᵢ are valine, threonine, and asparagine, respectively, the PPR motif can selectively bind to A (adenine);
(3-3) when the three amino acids A₁, A₄, and Lᵢᵢ are valine, asparagine, and asparagine, respectively, the PPR motif can selectively bind to C (cytosine);
(3-4) when the three amino acids A₁, A₄, and Lᵢᵢ are glutamic acid, glycine, and aspartic acid, respectively, the PPR motif can selectively bind to G (guanine);
(3-5) when the three amino acids A₁, A₄, and Lᵢᵢ are isoleucine, asparagine, and asparagine, respectively, the PPR motif can selectively bind to C or U;
(3-6) when the three amino acids A₁, A₄, and Lᵢᵢ are valine, threonine, and aspartic acid, respectively, the PPR motif can selectively bind to G;
(3-7) when the three amino acids A₁, A₄, and Lᵢᵢ are lysine, threonine, and aspartic acid, respectively, the PPR motif can selectively bind to G;
(3-8) when the three amino acids A₁, A₄, and Lᵢᵢ are phenylalanine, serine, and asparagine, respectively, the PPR motif can selectively bind to A;
(3-9) when the three amino acids A₁, A₄, and Lᵢᵢ are valine, asparagine, and serine, respectively, the PPR motif can selectively bind to C;
(3-10) when the three amino acids A₁, A₄, and Lᵢᵢ are phenylalanine, threonine, and asparagine, respectively, the PPR motif can selectively bind to A;
(3-11) when the three amino acids A₁, A₄, and Lᵢᵢ are isoleucine, asparagine, and aspartic acid, respectively, the PPR motif can selectively bind to U or A;
(3-12) when the three amino acids A₁, A₄, and Lᵢᵢ are threonine, threonine, and asparagine, respectively, the PPR motif can selectively bind to A;
(3-13) when the three amino acids A₁, A₄, and Lᵢᵢ are isoleucine, methionine, and aspartic acid, respectively, the PPR motif can selectively bind to U or C;
(3-14) when the three amino acids A₁, A₄, and Lᵢᵢ are phenylalanine, proline, and aspartic acid, respectively, the PPR motif can selectively bind to U;
(3-15) when the three amino acids A₁, A₄, and Lᵢᵢ are tyrosine, proline, and aspartic acid, respectively, the PPR motif can selectively bind to U; and
(3-16) when the three amino acids A₁, A₄, and Lᵢᵢ are leucine, threonine, and aspartic acid, respectively, the PPR motif can selectively bind to G.

[3] The method according to [1], wherein the combination of the two amino acids A₄ and Lᵢᵢ is a combination corresponding to the target RNA base or base sequence, and the combination of the amino acids is determined according to any one of the following propositions:
(2-1) when A₄ and Lᵢᵢ are asparagine and aspartic acid, respectively, the motif can selectively bind to U;
(2-2) when A₄ and Lᵢᵢ are asparagine and asparagine, respectively, the motif can selectively bind to C;
(2-3) when A₄ and Lᵢᵢ are threonine and asparagine, respectively, the motif can selectively bind to A;
(2-4) when A₄ and Lᵢᵢ are threonine and aspartic acid, respectively, the motif can selectively bind to G;
(2-5) when A₄ and Lᵢᵢ are serine and asparagine, respectively, the motif can selectively bind to A;
(2-6) when A₄ and Lᵢᵢ are glycine and aspartic acid, respectively, the motif can selectively bind to G;
(2-7) when A₄ and Lᵢᵢ are asparagine and serine, respectively, the motif can selectively bind to C;
(2-8) when A₄ and Lᵢᵢ are proline and aspartic acid, respectively, the motif can selectively bind to U;
(2-9) when A₄ and Lᵢᵢ are glycine and asparagine, respectively, the motif can selectively bind to A;
(2-10) when A₄ and Lᵢᵢ are methionine and aspartic acid, respectively, the motif can selectively bind to U;
(2-11) when A₄ and Lᵢᵢ are leucine and aspartic acid, respectively, the motif can selectively bind to C; and
(2-12) when A₄ and Lᵢᵢ are valine and threonine, respectively, the motif can selectively bind to U.

[4] A method for identifying a target base or base sequence for an RNA-binding protein comprising one or more (preferably 2 to 14) of the PPR motifs defined in [1], wherein:
the base or base sequence is identified by determining presence or absence of a base corresponding to a combination of the three amino acids A₁, A₄ and Lᵢᵢ of the PPR motifs, or a combination of the two amino acids A₄ and Lᵢᵢ of the PPR motifs on the basis of any of the propositions (3-1) to (3-16) mentioned in [2], or any of the propositions (2-1) to (2-12) mentioned in [3].

[5] A method for identifying a PPR protein that comprises one or more (preferably 2 to 14) of the PPR motifs defined in [1], and can bind to a target RNA base or a target RNA having a specific base sequence, wherein:
the PPR protein is identified by determining presence or absence of a combination of the three amino acids A₁, A₄ and Lᵢᵢ of the PPR motifs corresponding to the target RNA base or a specific base constituting the target RNA on the basis of any of the propositions (3-1) to (3-16) mentioned in [2], or any of the propositions (2-1) to (2-12) mentioned in [3].

[6] A method for controlling a function of RNA, comprising using a protein designed by the method according to [1].

[7] A complex comprising a region consisting of a protein designed by the method according to [1] and a functional region, which have been linked together.

[8] A method for modifying a cellular genetic material, which comprises the following steps:
preparing a cell containing an RNA having a target sequence; and
introducing the complex according to [7] into the cell, so that the protein region of the complex binds to the RNA having the target sequence, and therefore the functional region modifies the target sequence.

[9] A method for judging fertility of a gene of a PPR protein, which comprises:
the step of detecting amino acid polymorphism observed among various varieties for a gene of a PPR protein that functions as a fertility restoration factor for cytoplasmic male sterility;
the step of specifying relation of the polymorphism and the fertility for the gene; and
the step of specifying a base sequence of a gene of a PPR protein obtained from a test sample, and determining fertility of the test sample.

[10] The method according to [9], wherein the PPR protein is a protein comprising one or more (preferably 2 to 16) of PPR motifs each consisting of a polypeptide of 30- to 38-amino acid length represented by the formula 1 defined in [1].

[11] The method according to [9] or [10], wherein the amino acid polymorphism is specified as polymorphism observed in units of the PPR motifs.

[12] The method according to any one of [9] to [11], wherein the polymorphism observed in the PPR motifs is identified by a combination of the three amino acids A₁, A₄ and Lᵢᵢ, or a combination of the two amino acids A₄ and Lᵢᵢ of the motif of the formula 1.

[13] The method according to [12], wherein the polymorphism observed in the PPR motifs is identified as polymorphism of amino acid 4 (A₄) in the motifs of the formula 1.

[14] The method according to [13], wherein the fertility is indicated by the fact that amino acids 4 in all of the PPR motifs in the PPR protein are the same as amino acids 4 in all of the corresponding PPR motifs of Enko B, or the fact that the amino acids “ii” in all of the PPR motifs in the PPR protein are the same as the amino acids “ii” in all of the corresponding PPR motifs of Enko B.

[15] The method according to any one of [9] to [14], wherein the gene of the PPR protein is a family gene carried at the same locus as that of the “ORF687 gene” coding for Enko B, a gene coding for a protein showing an amino acid identity of 90% or higher to Enko B, or a gene showing a nucleotide sequence identity of 90% or higher to the “ORF687 gene” coding for Enko B.

[16] The method according to any one of [9] to [15], wherein the proteins encoded by the orf687-like genes of various varieties are any of the proteins of SEQ ID NOS: 576 to 578 and 585 to 591.

### Effect of the Invention

According to the present invention, a PPR motif capable of binding to a target RNA base and a protein containing it can be provided. By using a plurality of PPR motifs, a protein capable of binding to a target RNA having an arbitrary sequence or length can be provided.

According to the present invention, a target RNA of an arbitrary PPR protein can be predicted and identified, and conversely, a PPR protein capable of binding to an arbitrary RNA can be predicted and identified. Prediction of such a target RNA sequence enhances the possibility of elucidating the genetic identity thereof and using it. For example, in the case of considering fertility as a function of the PPR protein according to the present invention, for an industrially useful gene of PPR protein such as those capable of functioning as a restoration factor for cytoplasmic male sterility, functionalities of various homologous genes thereof providing proteins that show amino acid polymorphism can be determined on the basis of the difference of the target RNA sequences thereof.

Further, a functional region can be bound to a PPR motif or PPR protein provided by the present invention to prepare a complex.

The present invention can further be utilized for a method of delivering the aforementioned complex to a living body and allowing it to function, preparation of a transformant using a nucleic acid sequence (DNA or RNA) coding for a protein obtained by the present invention, as well as specific modification, control, and impartation of a function in various scenes in organisms (cells, tissues, and individuals).

### BRIEF DESCRIPTION OF THE DRAWINGS

FIGS. 1A-1C show the conserved sequences and amino acid numbers of the PPR motif. FIG. 1A shows the amino acids constituting the PPR motif defined in the present invention, and the amino acid numbers thereof. FIG. 1B shows the positions of the three amino acids (1, 4, and “ii” (−2)) that control the binding base selecting property on the putative structure. FIG. 1C shows the positions of the amino acids on the putative structure. By using the total amino acid sequences of Arabidopsis thaliana CRR4 (SEQ ID NO: 6) and CRR21 (SEQ ID NO: 3) as the query sequences for the program PHYRE (http://www.sbg.bio.ic.ac.uk/phyre/), the putative structures were analyzed. As a result, the structures were predicted with high scores using O-GlucNAc transferase (1w3b) as the template (4.3e-17 and 4.7e-16, for CRR4 and CRR21). Among the structures, the 5th PPR motif of CRR4 (left figure), and the 8th PPR motif of CRR21 (right figure) are shown. The positions 1, 4, and “ii” (−2) are shown as sticks in magenta color (dark gray in monochromatic indication).

FIG. 2 shows the RNA-editing PPR proteins analyzed so far and the RNA-editing sites as targets thereof.

FIG. 3A shows the PPR motif sequences and amino acid numbers of Arabidopsis thaliana RNA-editing PPR proteins.

FIGS. 3B-3H each shows continuation of FIG. 3A.

FIGS. 4A-4E show the amino acids in the PPR motifs involved in the RNA recognition. FIG. 4A shows identification of amino acids having a binding nucleotide specifying capacity in the PPR motif. The PPR motifs of RNA-editing PPR protein are aligned with an RNA-editing site upstream sequence in various positions. The alignment was performed by arranging the sequences at a 1-motif to 1-nucleotide correspondence, in a contiguous linear manner. The alignment P1 was obtained by fitting the last PPR motif of the protein to the base 1 nucleotide before the editable C. The base sequence was then moved toward the right, 1 base at a time, to obtain the alignments P2 to P6. The squares represent PPR motifs, and the diamond represents additional motifs (E, E+, DYW) on the C terminus side. If amino acids at specific sites in the motif (for example, amino acids of the motifs indicated in green (dark gray in monochromatic indication)) are responsible for the RNA base recognition, low randomness can be expected for corresponding nucleotides in a specific alignment (lower figure on the right). Otherwise, high randomness is expected (upper figure on the right). FIG. 4B shows binding RNA base specifying capacities of amino acids 1, 4, and “ii” (−2). Low randomness between the amino acid and the base in each alignment is shown in terms of a P value. FIG. 4C shows binding RNA base specifying capacities of amino acids 1, 4, and “ii” (−2) for various classifications of nucleic acids. They are indicated in a similar manner to that of FIG. 4B. The nucleic acids are classified according to type of nucleobase, purine or pyrimidine (RY, A & G or U & C), and presence or absence of hydrogen bond groups (WS, A & U or G & C). FIG. 4D shows results of further detailed analysis of the binding base specifying capacities of the RNA recognition amino acids in the PPR motifs shown in FIG. 4C mentioned above. It was demonstrated that, in addition to that amino acid 4 mainly determines the type of the binding base, purine or pyrimidine (RY), the amino acid “ii” (−2) functions to determine the form of the nucleotide, amino form (A and C) or keto form (G and U) (MK) (FIG. 4D). FIG. 4E shows examples of RNA recognition codes (PPR codes) of several PPR motifs. The white letters indicates types of amino acids 1, 4, and “ii” (−2). The occurrence frequencies of the codes are indicated in the row of “No.”, and the occurrence frequencies of the corresponding nucleic acids are indicated in the rows of “Nucleotide frequency”.

FIG. 5 shows identification (examples) of the amino acids in the PPR motifs involved in the RNA recognition. The amino acids involved in the RNA recognition were searched for by using data sets of RNA bases corresponding the PPR motifs in each alignment. For example, by using data of RNA bases corresponding the PPR motifs in alignment P4, the binding RNA base specifying capacities of amino acids 4 and 5 were analyzed. For each alignment, data were first sorted according to the types of the amino acids, and the numbers of the RNA bases contained were calculated (upper left table). Then, theoretical values of the numbers were prepared on the basis of the medians of the occurrence frequencies of all the RNA bases contained in the data sets (upper right table). By the chi square test using these two kinds of data, P values were calculated. The upper tables show the analysis results for amino acid 4 in alignment P4, for which significant P values were obtained, and the lower tables show the analysis results for the amino acid 5 in alignment P4, for which significant P values were not obtained.

FIGS. 6A-6C show results of search for the amino acids responsible for the RNA base specifying capacity. FIGS. 6A and 6B show P values for low randomness between the type of amino acid and the occurrence frequency of base calculated for the amino acids of all the positions in the alignments P1 to P6. The amino acids that showed significant P values (P<0.01) are indicated in magenta color (dark gray in monochromatic indication). The lines (horizontal lines in the graphs) in cyan color (dark gray in monochromatic indication) indicate P value of 0.01.

FIG. 6C shows the summary of the low randomness for each alignment. A product of the P values of the amino acids of the positions shown in FIGS. 6A-6B for each alignment is shown as a total value of the low randomness for that alignment.

FIG. 7 shows the binding RNA base specifying capacities exerted by two amino acids. The binding RNA base specifying capacities exerted by different combinations of two amino acids (amino acids 1 and 4, 1 and “ii”, and 4 and “ii”) were analyzed on the basis of low randomness of amino acids and corresponding bases, and the results are shown in the same manner as that used in FIGS. 4A-4E.

FIG. 8 shows the RNA recognition codes of the PPR motifs extracted from Arabidopsis thaliana.

FIG. 9 shows the sequences of Physcomitrella patens subsp. patens RNA-editing PPR proteins and the RNA-editing sites on which the proteins act. Together with the motif structures of the proteins, the sequences of amino acids 1, 4, and “ii” (−2) in each PPR motif are shown. The letters in magenta and cyan colors (both are in dark gray in monochromatic indication) show the combinations of amino acids homologous to the triPPR or diPPR codes extracted from Arabidopsis thaliana. The additional motifs (E, E+, DYW) on the C terminus side are also shown. The sequences of the RNA-editing sites on which the proteins act (upstream sequences containing editable C) are shown in terms of the positions in alignment P4 shown in FIG. 4.

FIG. 10 shows a flowchart of a method for calculating matching score between a PPR protein and an RNA-editing site RNA sequence. From the Uniprot or PROSITE database, PPR models of proteins are obtained, and the amino acid numbers are given according to FIG. 1. Amino acids 1, 4, and “ii” are extracted. As an example, the moss PPR protein, PpPPR71, is shown. Then, the matching combinations of amino acids are converted into a triPPR code matrix. The motifs that could not be converted into the triPPR codes are then converted into a diPPR code matrix. In parallel, the RNA-editing site 30 nt (the last nucleotide is the editable C) are converted into an expression matrix. As an example, there is shown the ccmFCeU122SF sequence, on which the PpPPR71 protein acts. Then, products of numbers of corresponding grids of the protein code matrix and the RNA expression matrix are obtained, and matching scores are calculated from the sum of them. The last line of the protein code matrix should be matched to the line corresponding to the base 4 nucleotide before the editable C. This calculation is performed for protein code matrixes prepared from the triPPR codes and the diPPR codes. A provisional P value for each RNA sequence is calculated with each of the triPPR codes and diPPR codes using a normal distribution curve prepared from matching scores for a plurality of RNA sequences. The final matching score (P value) is calculated as a product of the provisional P values of the triPPR and diPPR codes.

FIGS. 11A and 11B show prediction of the target RNA sequences of the PPR proteins using the PPR codes. FIG. 11A shows the matching scores for the RNA-editing sites of the triPPR or the diPPR codes obtained by conversion of amino acids 1, 4, and “ii” (−2) extracted from the moss PPR proteins as shown in FIG. 10, which values are shown in terms of P values. As the RNA-editing sites, 13 RNA-editing sites of the moss were used, and as reference sequences, 34 RNA-editing sites of Arabidopsis thaliana chloroplast were used. In the drawing, only the matching scores for the 13 RNA-editing sites of the moss are shown. The diamonds indicate matching scores of the proteins for the respective editing sites. The correct editing sites are shown in magenta color (solid gray in monochromatic indication). FIG. 11B shows the P values shown in FIG. 11A in the form of table.

FIGS. 12A and 12B show verification of accuracy for prediction of RNA-editing sites using Arabidopsis thaliana RNA-editing proteins. The prediction accuracy was verified by using the Arabidopsis thaliana PPR proteins used for the code extraction. FIG. 12A shows prediction of RNA-editing sites of 13 known PPR proteins with respect to the total 34 chloroplast RNA-editing sites. The diamonds indicate the matching scores between the proteins and the RNA-editing site sequences. The correct RNA-editing sites are shown in magenta color (solid gray in monochromatic indication). FIG. 12B shows prediction of RNA-editing sites of 11 known PPR proteins with respect to the total 488 mitochondria RNA-editing sites.

FIGS. 13A-13D show prediction of the target RNA editing sites of Arabidopsis thaliana PPR protein AHG11, and experimental verification thereof. FIG. 13A shows the motif structure of AHG11. It has a typical structure of RNA-editing PPR protein comprising 12 PPR motifs and the additional motifs (E, E+, DYW) on the C terminus side. In Ahg11 mutants, there can be found a new translation stop codon in the coding region generated by the point mutation at the position indicated with the asterisk (295 Trp). FIG. 13B shows prediction of the target RNA-editing sites using all the RNA-editing sites contained in the chloroplasts and mitochondria of Arabidopsis thaliana. The top ten editing sites that showed the highest P values are shown. Presence or absence of the RNA editing in wild strain and mutant strain was experimentally verified, and the results are shown in the column of Editing status. The sites for which RNA editing was detected in both the wild strain and the mutant strain are indicated as E, and the site for which RNA editing could not observed only in the mutant strain is indicated as Un. FIG. 13C shows the results of the prediction in the form of graph. FIG. 13D shows experimental verification of the target RNA-editing sites of AHG11. There are shown the results of the sequence analysis of the region containing the mitochondria nad4. RNAs were extracted from the wild strain and the ahg11 mutant strain, cDNAs were prepared by reverse transcription, and nucleotide sequence analysis of them was conducted. There are two RNA-editing sites (nsd4_362 and _376) in this region. The edited sites are indicated with black arrows, and the non-edited site is indicated with a white arrow.

FIG. 14 shows prediction of the target sites in the chloroplast genome sequence. The target sites were predicted in the Arabidopsis thaliana chloroplast total genome sequence (154,478 bp) by using six PPR proteins. For the prediction, the codes extracted from Arabidopsis thaliana (At codes) or the codes extracted from Arabidopsis thaliana and the moss (At+Pp codes) were used.

FIG. 15 shows the RNA recognition codes of the PPR motifs extracted from Arabidopsis thaliana and Physcomitrella patens subsp. patens.

FIGS. 16A-16HH show amino acid sequences or nucleotide sequences that are referenced in the present disclosure and/or relevant to the present invention.

FIGS. 17A and 17B show analysis of the binding of the Enko B protein and RNA containing the cytoplasmic male sterility (CMS) gene. FIG. 17A shows a schematic diagram around the mitochondrial orf125, and also schematically shows the regions of RNAa, RNAbc, RNAb, and RNAc used in the binding experiment. FIG. 17B shows binding of the Enko B protein and RNA. Enko B protein (1.4 nmol) and ³²P-labeled RNAbc (0.1 ng) were reacted in the presence of non-labeled RNAa, RNAbc, RNAb, and RNAc (×5 and ×10 w/w with respect to RNAbc, used as a competitive inhibition substance) in 20 μL of a reaction mixture to perform the gel shift competition experiment.

FIGS. 18A-18C show binding of the ORF687-like proteins and RNA. FIG. 18A shows the results of analysis of RNA binding characteristics of ORF687-like proteins performed by gel shift assay for binding of Enko B (Rf), Kosena B (rf), and Enko A (rf) with RNAb. FIG. 18B is shows the results of FIG. 18A in the form of graph, and dissociation constants (KD) of the proteins representing the RNA binding capacities thereof were calculated on the basis of this graph. FIG. 18C shows the results of calculation of the matching scores of Enko B (Rf), Kosena B (rf), and Enko A (rf), and potential binding sites thereof performed in the same manner as that used for obtaining the results shown in FIGS. 19A-19B.

FIGS. 19A and 19B show prediction of binding sequence of the fertility restoration factor that acts on Ogura-type cytoplasm. FIG. 19A shows the results for prediction of binding of the Enko B protein using the PPR codes, and the structure of RNA containing the CMS gene orf125 is shown in the lower diagram of FIG. 19A. As for the regions from RNAa to RNAc shown in FIG. 19A, refer to FIGS. 17A-17B. In FIG. 19A, the regions of Nos. 208, 230, 316, 352 and 373 are focused on, among the regions that showed a significantly high P value (FIG. 19A).

FIGS. 20A-20C show the secondary structure and structural change of the candidate binding RNA region of ORF687-like protein. FIG. 20A shows the secondary structure of the region including the region of No. 306 and the predicted binding sites for the ORF687-like protein, and shows PPR motifs with boxes together with the corresponding bases. The 2nd and 3rd PPR motifs for which Enko B (Rf) and Kosena B (rf) show a remarkable difference are emphasized. FIG. 20B shows the secondary structure of the region including the regions of Nos. 352 and 373 and the predicted binding sites for the ORF687-like protein. FIG. 20C shows results indicating structural change of RNAb induced by Enko B, which were obtained by mixing RNAb and Enko B protein, and then adding a double-strand selective RNase (RNase V1).

FIG. 21A shows alignment of ORF687-like proteins.

FIG. 21B shows alignment of ORF687-like proteins.

FIG. 22 shows a list of the base specifying amino acids of ORF687-like proteins contained in various radish varieties.

### DESCRIPTION OF EMBODIMENTS

### [PPR Motif and PPR Protein]

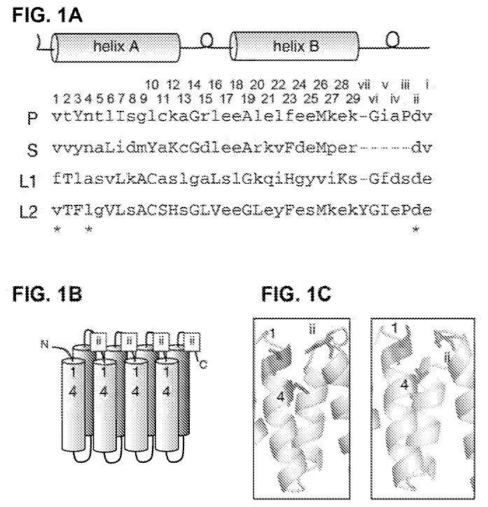

The term “PPR motif” used in the present invention refers to a polypeptide consisting of 30 to 38 amino acids and having an amino acid sequence showing an E value determined by amino acid sequence analysis using a protein domain search program on the Web, i.e., an E value obtained by using Pfam, PF01535, or Prosite, PS51375, not larger than a predetermined value (desirably E-03), unless especially indicated. The position numbers of amino acids constituting the PPR motif defined in the present invention are substantially synonymous with those obtainable with PF01535, but they correspond to those obtained by subtracting 2 from the numbers of the amino acid positions obtained with PS51375 (for example, the position 1 referred to in the present invention is the position 3 obtained with PS51375). Further, the amino acid “ii” (−2) is the second amino acid from the end (C-terminus side) of the amino acids constituting the PPR motif, or the second amino acid towards the N-terminus side from the first amino acid of the following PPR motif, i.e., -2nd amino acid (FIG. 1). When the following PPR motif is not definitely identified, the amino acid 2 amino acids before the first amino acid of the following helical structure is the amino acid “ii”. For Pfam, http://pfam.sanger.ac.uk/ can be referred to, and for Prosite, http://www.expasy.org/prosite/ can be referred to.

Although the conservativeness of the conserved amino acid sequence of the PPR motif is low at the amino acid level, two of the α-helixes as the secondary structure are well conserved. Although a typical PPR motif is constituted by 35 amino acids, the length thereof is as variable as 30 to 38 amino acids.

More specifically, the PPR motif referred to in the present invention consists of a polypeptide of a 30- to 38-amino acid length represented by the formula 1.

[F4]

(HelixA)-X-(HelixB)-L (Formula 1)

In the formula:
Helix A is a moiety of 12-amino acid length capable of forming an α-helix structure, and is represented by the formula 2;

[F5]

A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid;
X does not exist, or is a moiety of 1- to 9-amino acid length;
Helix B is a moiety of 11- to 13-amino acid length capable of forming an α-helix structure; and
L is a moiety of 2- to 7-amino acid length represented by the formula 3;

[F6]

Lᵥᵢᵢᵢ-Lᵥᵢ-Lᵥ-Lᵢᵥ-Lᵢᵢᵢ-Lᵢᵢ-Lᵢ (Formula 3)
wherein, in the formula 3, the amino acids are numbered “i” (−1), “ii” (−2), and so on from the C-terminus side,
provided that Lᵢᵢᵢ to Lᵥᵢᵢ may not exist.
   The term “PPR protein” used in the present invention refers to a PPR protein comprising one or more, preferably two or more, of the above-mentioned PPR motifs, unless especially indicated. The term “protein” used in this specification refers to any substance consisting of a polypeptide (chain consisting of a plurality of amino acids bound via peptide bonds), unless especially indicated, and includes those consisting of a polypeptide of a comparatively low molecular weight. The term “amino acid” used in the present invention refers to a usual amino acid molecule, and also refers to an amino acid residue constituting a peptide chain. Which one is referred to shall be clear to those skilled in the art from the context.

Many kinds of PPR proteins exist in plants, and in the case of Arabidopsis thaliana, about 500 kinds of proteins and about 5000 kinds of the motifs can be found. Also in many land plants, such as rice plant, poplar, and selaginella, PPR motifs and PPR proteins of various amino acid sequences exist. It is known that some PPR proteins are important factors for obtaining F1 seeds for hybrid vigor as a fertility restoration factor that works for pollen (male gamete) formation. As an action analogous to the fertility restoration, it has been clarified that some PPR proteins work for speciation. It has also been clarified that most of PPR proteins act on RNA in mitochondria or chloroplasts.

For animals, it is known that anomaly of the PPR protein identified as LRPPRC causes Leigh syndrome French Canadian type (LSFC, Leigh's syndrome, subacute necrotizing encephalomyelopathy).

The term “selectively” used in the present invention concerning the binding property of the PPR motif with RNA base means that the binding activity for one base among the RNA bases is higher than the binding activities for the other bases, unless otherwise indicated. Concerning this selectivity, those skilled in the art can plan and conduct an experiment for confirming it, and it can also be obtained by calculation as disclosed in the examples described in this specification. The term RNA base used in the present invention refers to a base of a ribonucleotide constituting RNA, specifically, any one of adenine (A), guanine (G), cytosine (C), and uracil (U). The PPR protein may have selectivity for a base in RNA, but it does not bind to a nucleic acid monomer. Although the sequence searching method for the conserved amino acids as the PPR motif had been established before the present invention was accomplished, the correspondence between the amino acid and the selective binding with RNA base was not discovered at all.

The present invention provides the following findings.

(I) Information concerning positions of amino acids important for the selective binding: Specifically, combination of the three amino acids, amino acids 1, 4, and “ii” (−1) (A₁, A₄, Lᵢᵢ), or combination of the two amino acids, amino acids 4 and “ii” (−1) (A₄, Lᵢᵢ), is important for the selective binding with an RNA base, and to which RNA base the motif binds is determined by such a combination.

The present invention is based on the findings concerning combination of the three amino acids A₁, A₄, and Lᵢᵢ, and/or combination of the two amino acids A₄, and Lᵢᵢ found by the inventors of the present invention.

(II) Information concerning the correspondence of combination of the three amino acids of A₁, A₄, and Lᵢᵢ and RNA base: Specifically, the followings are mentioned.
(3-1) When the combination of the three amino acids of A₁, A₄, and Lᵢᵢ is a combination of valine, asparagine, and aspartic acid as A₁, A₄, and Lᵢᵢ, respectively, the PPR motif has a selective RNA base binding capacity that it strongly binds to U, less strongly binds to C, and still less strongly binds to A or G.
(3-2) When the combination of the three amino acids of A₁, A₄, and Lᵢᵢ is a combination of valine, threonine, and asparagine as A₁, A₄, and Lᵢᵢ, respectively, the PPR motif has a selective RNA base binding capacity that it strongly binds to A, less strongly binds to G, and still less strongly binds to C, but dose not binds to U.
(3-3) When the combination of the three amino acids of A₁, A₄, and Lᵢᵢ is a combination of valine, asparagine, and asparagine as A₁, A₄, and Lᵢᵢ, respectively, the PPR motif has a selective RNA base binding capacity that it strongly binds to C, and less strongly binds to A or U, but does not bind to G.
(3-4) When the combination of the three amino acids of A₁, A₄, and Lᵢᵢ is a combination of glutamic acid, glycine, and aspartic acid as A₁, A₄, and Lᵢᵢ, respectively, the PPR motif has a selective RNA base binding capacity that it strongly binds to G, but does not bind to A, U, and C.
(3-5) When the combination of the three amino acids of A₁, A₄, and Lᵢᵢ is a combination of isoleucine, asparagine, and asparagine as A₁, A₄, and Lᵢᵢ, respectively, the PPR motif has a selective RNA base binding capacity that it strongly binds to C, less strongly binds to U, and still less strongly binds to A, but does not bind to G.
(3-6) When the combination of the three amino acids of A₁, A₄, and Lᵢᵢ is a combination of valine, threonine, and aspartic acid as A₁, A₄, and Lᵢᵢ, respectively, the PPR motif has a selective RNA base binding capacity that it strongly binds to G, and less strongly binds to U, but does not bind to A and C.
(3-7) When the combination of the three amino acids of A₁, A₄, and Lᵢᵢ is a combination of lysine, threonine, and aspartic acid as A₁, A₄, and Lᵢᵢ, respectively, the PPR motif has a selective RNA base binding capacity that it strongly binds to G, and less strongly binds to A, but does not bind to U and C.
(3-8) When the combination of the three amino acids of A₁, A₄, and Lᵢᵢ is a combination of phenylalanine, serine, and asparagine as A₁, A₄, and Lᵢᵢ, respectively, the PPR motif has a selective RNA base binding capacity that it strongly binds to A, less strongly binds to C, and still less strongly binds to G and U.
(3-9) When the combination of the three amino acids of A₁, A₄, and Lᵢᵢ is a combination of valine, asparagine, and serine as A₁, A₄, and Lᵢᵢ, respectively, the PPR motif has a selective RNA base binding capacity that it strongly binds to C, and less strongly binds to U, but does not bind to A and G.
(3-10) When the combination of the three amino acids of A₁, A₄, and Lᵢᵢ is a combination of phenylalanine, threonine, and asparagine as A₁, A₄, and Lᵢᵢ, respectively, the PPR motif has a selective RNA base binding capacity that it strongly binds to A, but does not bind to G, U, and C.
(3-11) When the combination of the three amino acids of A₁, A₄, and Lᵢᵢ is a combination of isoleucine, asparagine, and aspartic acid as A₁, A₄, and Lᵢᵢ, respectively, the PPR motif has a selective RNA base binding capacity that it strongly binds to U, and less strongly binds to A, but does not bind to G and C.
(3-12) When the combination of the three amino acids of A₁, A₄, and Lᵢᵢ is a combination of threonine, threonine, and asparagine as A₁, A₄, and Lᵢᵢ, respectively, the PPR motif has a selective RNA base binding capacity that it strongly binds to A, but does not bind to G, U, and C.
(3-13) When the combination of the three amino acids of A₁, A₄, and Lᵢᵢ is a combination of isoleucine, methionine, and aspartic acid as A₁, A₄, and Lᵢᵢ, respectively, the PPR motif has a selective RNA base binding capacity that it strongly binds to U, and less strongly binds to C, but does not bind to A and G.
(3-14) When the combination of the three amino acids of A₁, A₄, and Lᵢᵢ is a combination of phenylalanine, proline, and aspartic acid as A₁, A₄, and Lᵢᵢ, respectively, the PPR motif has a selective RNA base binding capacity that it strongly binds to U, and less strongly binds to C, but does not bind to A and G.
(3-15) When the combination of the three amino acids of A₁, A₄, and Lᵢᵢ is a combination of tyrosine, proline, and aspartic acid as A₁, A₄, and Lᵢᵢ, respectively, the PPR motif has a selective RNA base binding capacity that it strongly binds to U, but does not bind to A, G, and C.
(3-16) When the combination of the three amino acids of A₁, A₄, and Lᵢᵢ is a combination of leucine, threonine, and aspartic acid as A₁, A₄, and Lᵢᵢ, respectively, the PPR motif has a selective RNA base binding capacity that it strongly binds to G, but does not bind to A, U, and C.

(II) Information concerning the correspondence of combination of the two amino acids of A₄, and Lᵢᵢ and RNA base: Specifically, the followings are mentioned.
(2-1) When A₄ and Lᵢᵢ are asparagine and aspartic acid, respectively, the PPR motif has a selective RNA base binding capacity that it strongly binds to U, less strongly binds to C, and still less strongly binds to A and G.
(2-2) When A₄ and Lᵢᵢ are asparagine and asparagine, respectively, the PPR motif has a selective RNA base binding capacity that it strongly binds to C, less strongly binds to U, and still less strongly binds to A and G.
(2-3) When A₄ and Lᵢᵢ are threonine and asparagine, respectively, the PPR motif has a selective RNA base binding capacity that it strongly binds to A, and weakly binds to G, U, and C.
(2-4) When A₄ and Lᵢᵢ are threonine and aspartic acid, respectively, the PPR motif has a selective RNA base binding capacity that it strongly binds to G, and weakly binds to A, U, and C.
(2-5) When A₄ and Lᵢᵢ are serine and asparagine, respectively, the PPR motif has a selective RNA base binding capacity that it strongly binds to A, and less strongly binds to G, U, and C.
(2-6) When A₄ and Lᵢᵢ are glycine and aspartic acid, respectively, the PPR motif has a selective RNA base binding capacity that it strongly binds to G, less strongly binds to U, and still less strongly binds to A, but does not bind to C.
(2-7) When A₄ and Lᵢᵢ are asparagine and serine, respectively, the PPR motif has a selective RNA base binding capacity that it strongly binds to C, less strongly binds to U, and still less strongly binds to A and G.
(2-8) When A₄ and Lᵢᵢ are proline and aspartic acid, respectively, the PPR motif has a selective RNA base binding capacity that it strongly binds to U, and less strongly binds to G and C, but does not bind to A.
(2-9) When A₄ and Lᵢᵢ are glycine and asparagine, respectively, the PPR motif has a selective RNA base binding capacity that it strongly binds to A, and less strongly binds to G, but does not bind to C and U.
(2-10) When A₄ and Lᵢᵢ are methionine and aspartic acid, respectively, the PPR motif has a selective RNA base binding capacity that it strongly binds to U, and weakly binds to A, G, and C.
(2-11) When A₄ and Lᵢᵢ are leucine and aspartic acid, respectively, the PPR motif has a selective RNA base binding capacity that it strongly binds to C, and less strongly binds to U, but does not bind to A and G.
(2-12) When A₄ and Lᵢᵢ are valine and threonine, respectively, the PPR motif has a selective RNA base binding capacity that it strongly binds to U, and less strongly binds to A, but does not bind to G and C.

In the examples described in this specification, binding of proteins partially analyzed genetically or molecular biologically and potential RNA target sequences thereof are further analyzed by computational science techniques to obtain the aforementioned findings. More precisely, binding or selective binding of the proteins and RNA is analyzed on the basis of P value (probability) as an index. According to the present invention, when the P value is 0.05 or smaller (contingency of 5% or less), which means a generally significant level, preferably when the P value is 0.01 or smaller (contingency of 1% or less), more preferably when a more significant P value compared with the foregoing levels is calculated, it is evaluated that the probability for binding of the protein and RNA is sufficiently high. Such judgment based on the P value can fully be understood by those skilled in the art.

Binding property of a specific combination of amino acids at specific positions for an RNA base can be experimentally confirmed. Experiments for such a purpose include preparation of a PPR motif or a protein containing a plurality of PPR motifs, preparation of a substrate RNA, and test for the binding property (for example, gel shift assay). These experiments are well known to those skilled in the art, and for specific procedures and conditions for them, Patent document 2, for example, can be referred to.

### [Use of PPR Motif and PPR Protein]

### Identification and Design:

One PPR motif can recognize a specific base of RNA. Further, according to the present invention, by choosing amino acids of specific positions, PPR motifs that selectively recognize each of A, U, G, and C can be selected or designed, and a protein containing an appropriate series of such PPR motifs can recognize a corresponding specific sequence. Therefore, according to the present invention, a natural PPR protein that selectively binds to RNA having a specific base sequence can be predicted and identified, and conversely, RNA that serves as a target of binding of a PPR protein can be predicted and identified. The prediction and identification of such a target is useful for elucidating genetic identity thereof, and expands availability of the target. Further, according to the present invention, a PPR motif that can selectively bind to a desired RNA base, and a protein comprising a plurality of PPR motifs that can sequence-specifically bind to a desired RNA can be designed. For designing moieties other than the amino acids of the important positions in the PPR motif, sequence information of natural PPR motifs can be referred to. Further, such a PPR motif or protein as mentioned above can also be designed by replacing only the amino acids of the positions of interest in the whole sequence of a natural PPR motif or protein. Although the number of repetition times of the PPR motif can be appropriately chosen depending on the target sequence, it may be, for example, 2 or more, or 2 to 20.

At the time of the designing, types of amino acids other than those of the combination of amino acids 1, 4, and “ii” or amino acids 4, and “ii” may be taken into consideration. For example, types of the 8th and 12th amino acids described in Patent document 2 mentioned above may be important for expression of the RNA binding activity. According to the study of the inventors of the present invention, A₈ of a certain PPR motif and A₁₂ of the same PPR motif may cooperate for binding to RNA. As may be a basic amino acid, preferably lysine, or an acidic amino acid, preferably aspartic acid, and A₁₂ may be a basic amino acid, a neutral amino acid, or a hydrophobic amino acid.

The designed motif or protein can be prepared by the methods well known to those skilled in the art. That is, the present invention provides a PPR motif that selectively binds to a specific RNA base, and a PPR protein that specifically binds to RNA having a specific sequence, which are designed by paying attention to the combination of amino acids 1, 4, and “ii” or the combination of amino acids 4 and “ii”. In particular, it was found that, for the action on fertility as a function of the PPR protein, amino acid 4 (A₄) and the amino acid “ii” are effective for both the cases of the aforementioned combination of three amino acids and combination of two amino acids. Such a motif and protein can be prepared by the methods well known to those skilled in the art, even in a relatively large amount, and such methods may comprise determining a nucleic acid sequence coding for an amino acid sequence of an objective motif or protein from that amino acid sequence, cloning it, and preparing a transformant that produces the objective motif or protein.

### Preparation of Complex and Use Thereof:

The PPR motif or PPR protein provided by the present invention can be made into a complex by binding a functional region. The functional region means a moiety having a specific biological function such as enzymatic function, catalytic function, inhibition function, and promotion function exerted in living bodies or cells, or a moiety having a function as a marker. Such a region consists of, for example, a protein, peptide, nucleic acid, physiologically active substance, or drug. Examples of protein as the functional region include ribonuclease (RNase). Examples of RNase include RNase A (for example, bovine pancreatic ribonuclease A, PDB 2AAS) and RNase H. Such a complex does not exist in the nature, and it is a novel substance.

Further, the complex provided by the present invention may be able to deliver the functional region to a living body or cell in an RNA sequence-specific manner, and allow it to function. It may be therefore able to modify or disrupt RNA, or impart a novel function to RNA, in a living body or cell in an RNA sequence-specific manner, like the zinc finger proteins (Non-patent document 1 mentioned above) or TAL effector (Non-patent document 2 and Patent document 1 mentioned above). Furthermore, it may be able to deliver a drug to RNA in an RNA sequence-specific manner. Therefore, the present invention provides a method for delivering a functional material in an RNA sequence-specific manner.

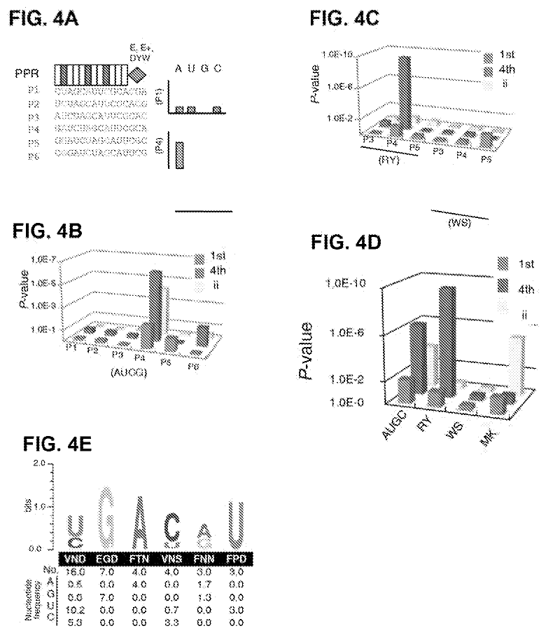

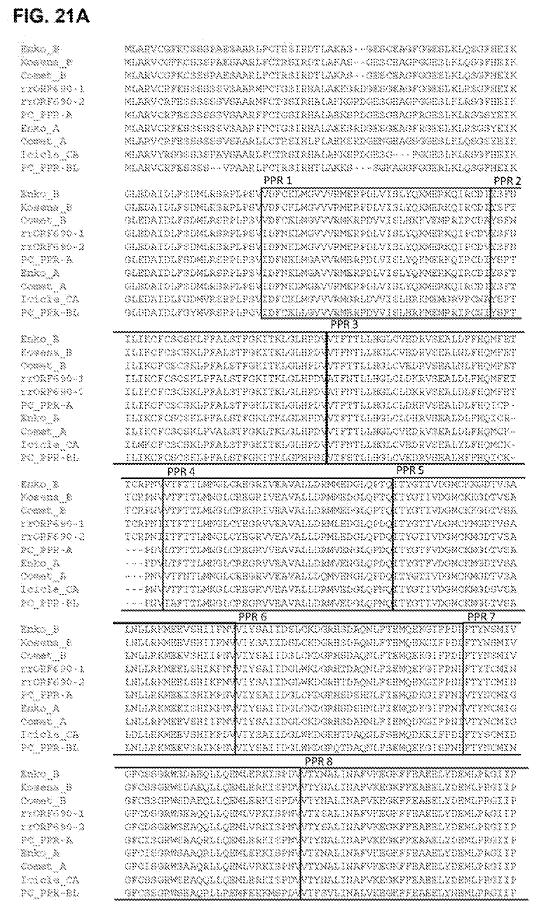

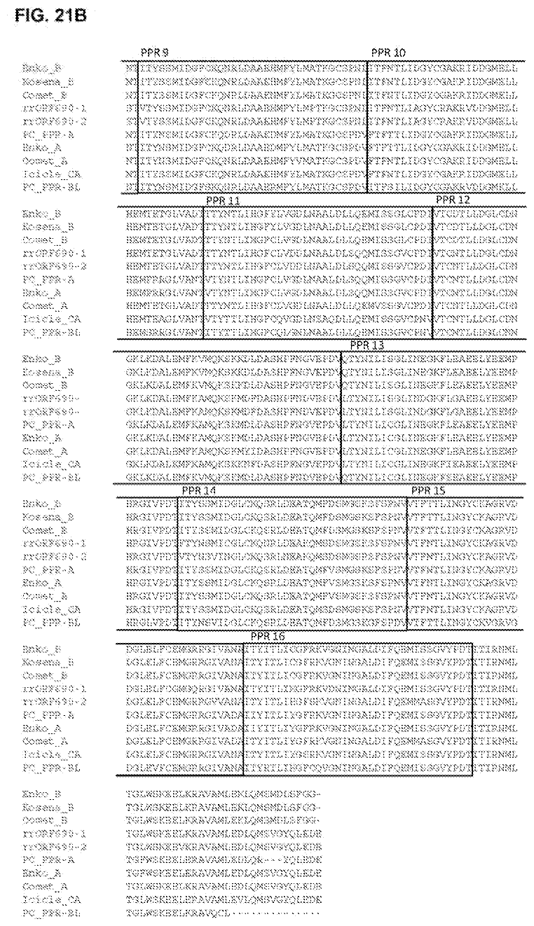

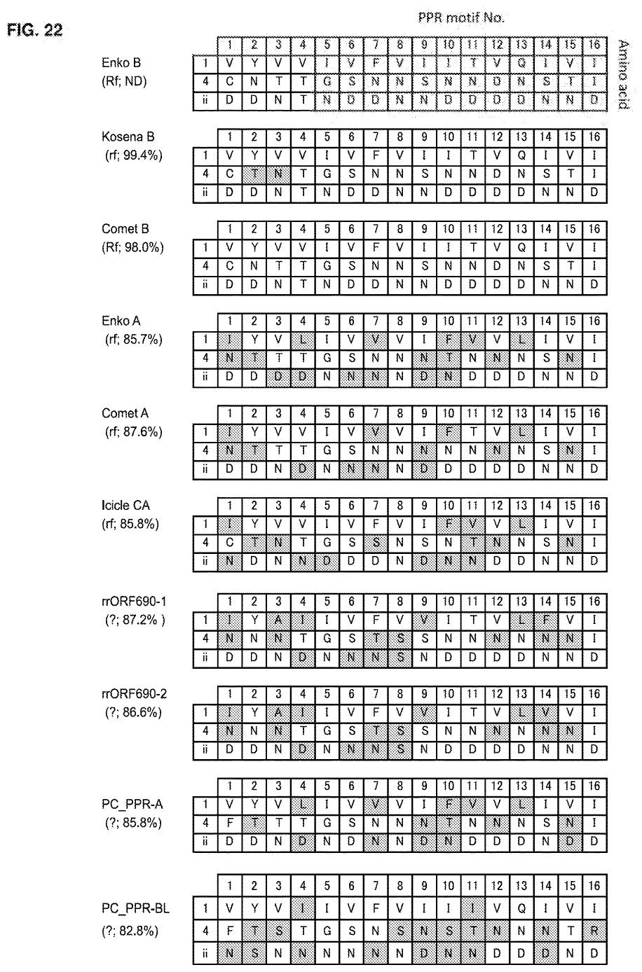

It is known that some PPR proteins are important for obtaining F1 seeds for hybrid vigor as a fertility restoration factor that works for pollen (male gamete) formation. It is expected that a fertility restoration factor not identified yet can be identified, and a technique for highly utilize such a factor can be developed by the present invention. For example, as elucidated in the examples described in this specification, if amino acid polymorphism is detected for a gene for a specific PPR motif in a PPR protein that works as a fertility restoration factor for cytoplasmic male sterility, and relation of the polymorphism and fertility is established for the gene, it can be judged whether the gene of the PPR protein in a test sample has a genotype relating to fertility or a genotype relating to sterility. Examples of the gene of the PPR protein in which the polymorphism is detected in such a case as mentioned above include, for example, in the case of radish, a family gene locating at the same locus as that of the “OFR687 gene” coding for the OFR687 protein of Enko (named Enko B), a gene coding for a protein showing an amino acid identity of 90% or higher to Enko B, and a gene showing a nucleotide sequence identity of 90% or higher to the “ORF687 gene” coding for Enko B. The family gene locating at the same locus as that of the “OFR687 gene” coding for the OFR687 protein of Enko (named Enko B) includes all the genes shown in FIGS. 21A-21B and 22 (Kosena B, Comet B, Enko A, Comet A, Icicle CA, rrORF690-1, rrORF690-2, PC_PPR-A, PC_PPR-BL), but it is not limited to these. The gene coding for a protein showing an amino acid identity of 90% or higher to Enko B, and the gene showing a nucleotide sequence identity of 90% or higher to the “ORF687 gene” coding for Enko B can be obtained by searching gene databases, and the species as the origin thereof is not limited to those of radish. The PPR motif is a PPR motif consisting of a polypeptide of 30- to 38-amino acid length represented by the formula 1 mentioned above, and the PPR protein may comprise one or more of such PPR motifs (preferably 2 to 16 motifs). As the polymorphism in the PPR motif, there can be used polymorphism of the combination of amino acids 1, 4, and “ii” or the combination of amino acids 4 and “ii”, which was elucidated to be responsible for the binding of PPR motif to RNA by the present invention. As seen from the P values shown in FIG. 4B or 4D, among the amino acids of the combinations responsible for the binding of the PPR motif to RNA, amino acid 4 plays the most important role, and the amino acid “ii” plays the secondarily important role. It was further elucidated that, in comparison with the PPR protein of Enko B, the fact that amino acids 4 of all the PPR motifs in a protein encoded by a gene as a test subject are the same as those of Enko B, or the fact that the amino acids “ii” in all the corresponding PPR motifs are the same as those of Enko B is important for the function as a fertility restoration factor. Further, it was also elucidated that, similarly to the fertility restoration, some PPR proteins act on speciation. It is expected that identification and modification of a target RNA of the PPR protein enable mating of species, of which mating has so far been impossible. Further, since most of the PPR proteins act on RNA in mitochondria and chloroplasts, the novel PPR proteins provided by the present invention will contribute to modification and improvement of the functions concerning photosynthesis, respiration, and synthesis of useful metabolites.

Further, for animals, it is known that anomaly of the PPR protein identified as LRPPRC causes Leigh syndrome French Canadian type (LSFC, Leigh's syndrome, subacute necrotizing encephalomyelopathy). The present invention can contribute to the treatment (prophylactic treatment, therapeutic treatment, suppression of advance) of LSFC.

Further, the PPR proteins are involved in all the steps of RNA processing seen in organelles, digestion, RNA editing, translation, splicing, and RNA stability. According to the present invention, it can be expected that, by modifying the binding base selectivity of a PPR motif, expression of a desired RNA can be modified.

The PPR proteins used in the present invention as materials mainly function for specification of the editing site of RNA editing (conversion of genetic information on RNA, C to U in many cases) (refer to References 2 and 3 mentioned later). The PPR proteins of this type have an additional motif suggested to interact with an RNA editing enzyme existing on the C-terminus side. It can be expected that, by using a PPR protein having such a structure, nucleotide polymorphism can be introduced, and a disease or condition induced by nucleotide polymorphism can be treated.

Further, a part of PPR proteins have an RNA cleavage enzyme on the C-terminus side. By modifying the binding RNA base selectivity of the PPR motif on the N terminus side of such a PPR protein, an RNA sequence-specific RNA cleaving enzyme can be constituted. Furthermore, a complex having a marker moiety such as GFP bound to a PPR protein can be used for visualizing a desired RNA in a living body.

Further, the existing PPR proteins include those that act on DNA. It has been reported that one of them is the transcription activator of a mitochondrial gene, and another one is a transcription activator localizing in the nucleus. Therefore, it may also be possible to design a protein factor that binds to a desired DNA sequence on the basis of the findings obtained by the present invention.

### EXAMPLES

### Example 1: Collection of PPR Proteins Involved in RNA Editing and Target Sequences Thereof

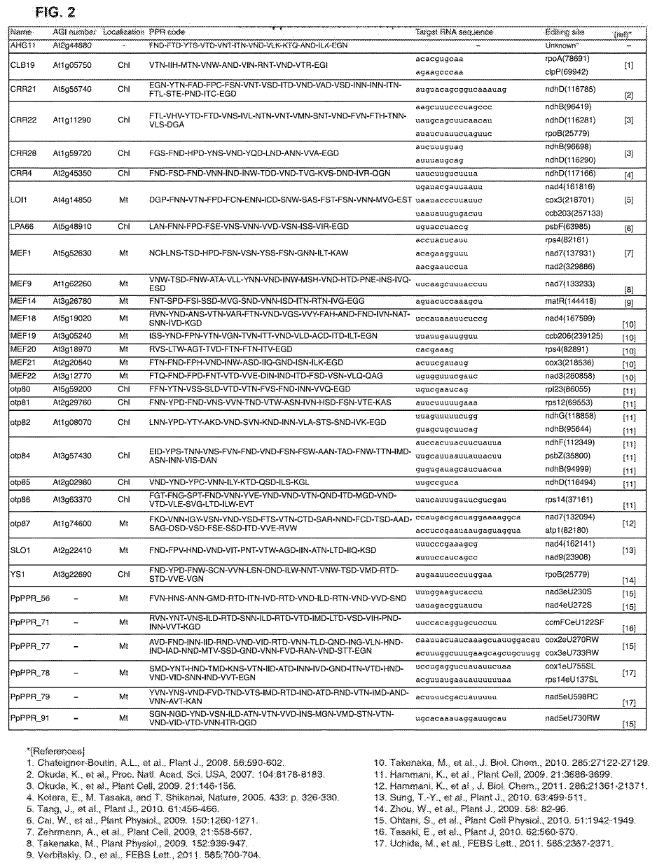

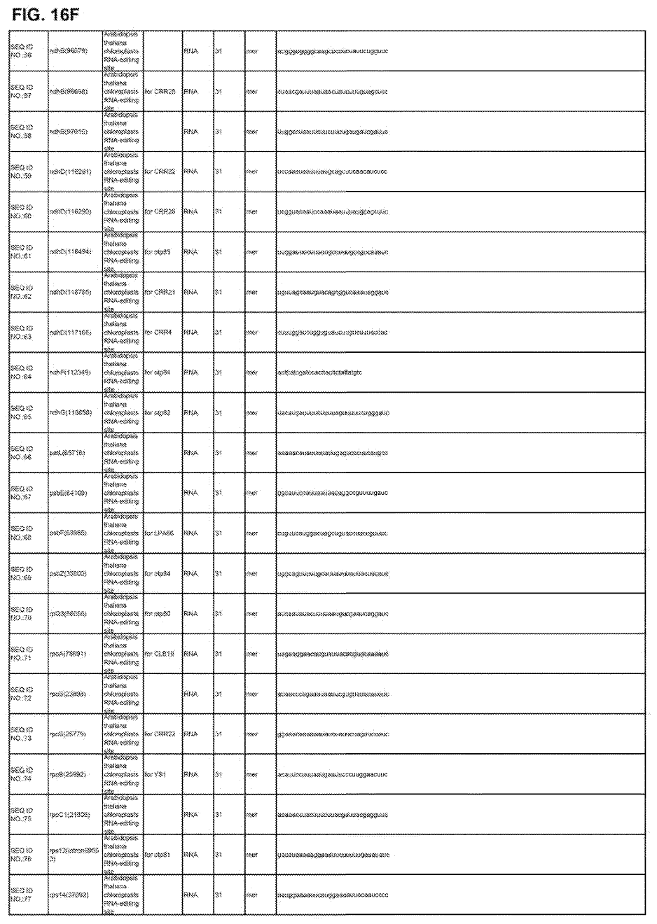

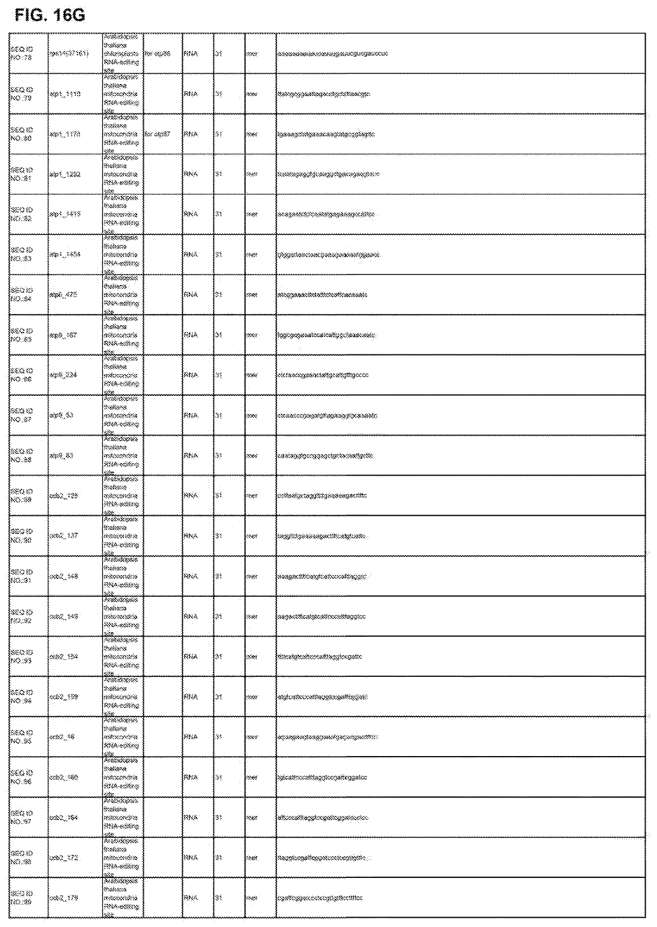

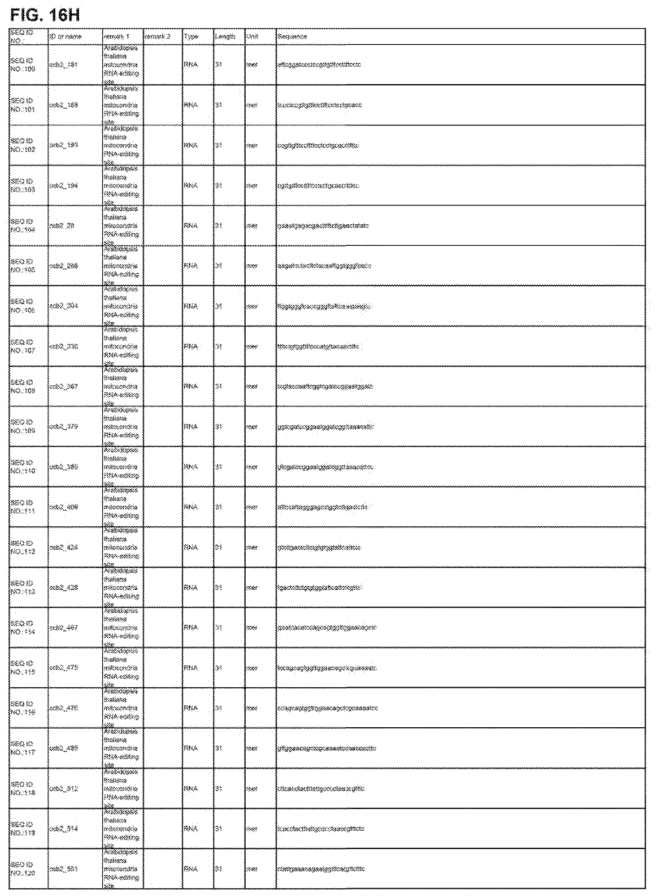

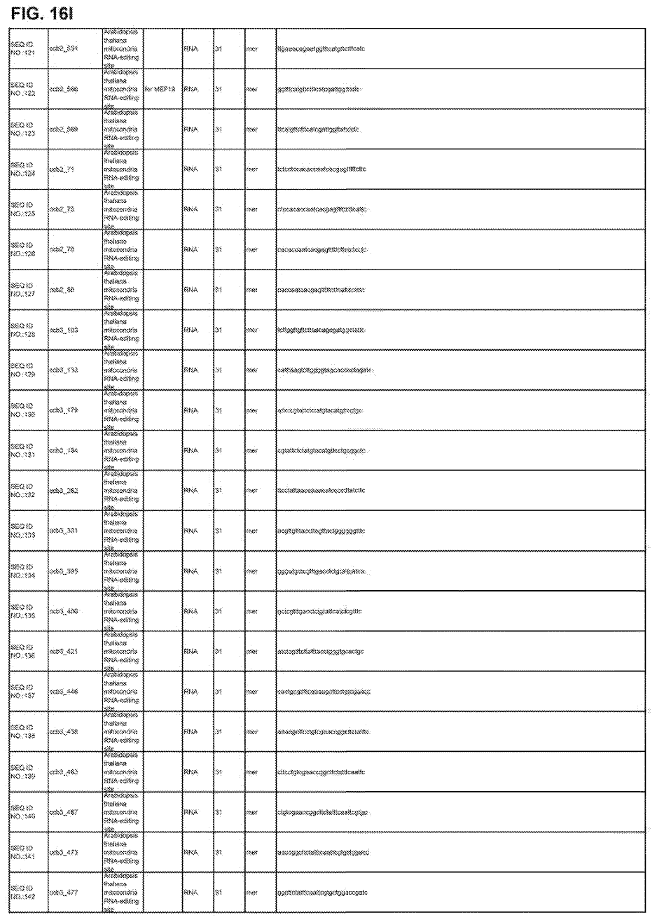

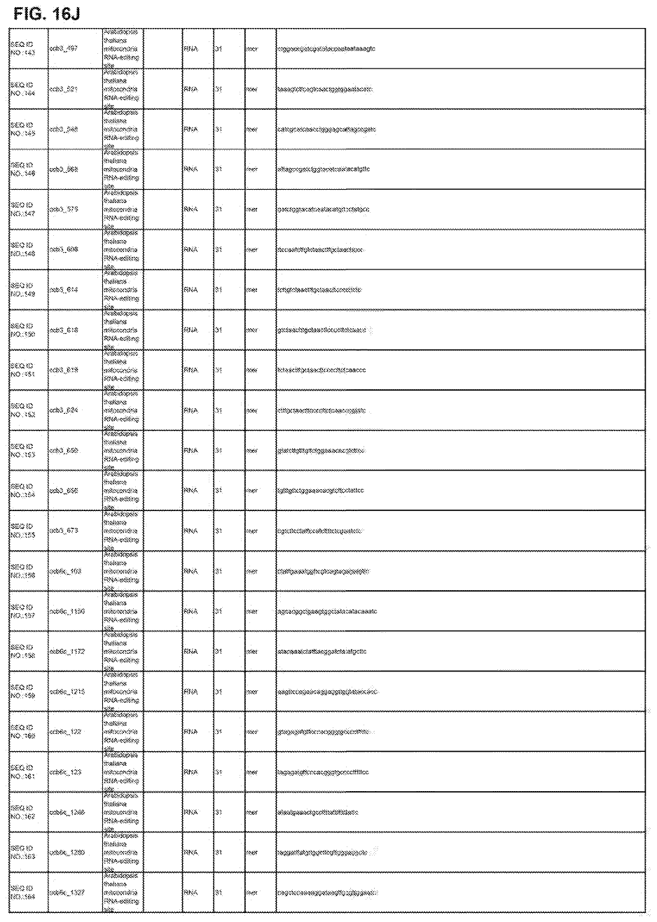

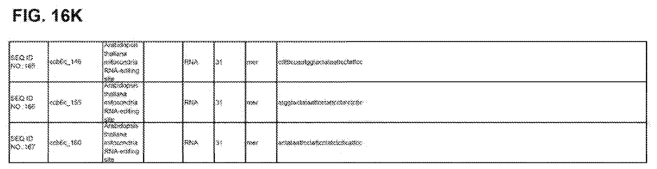

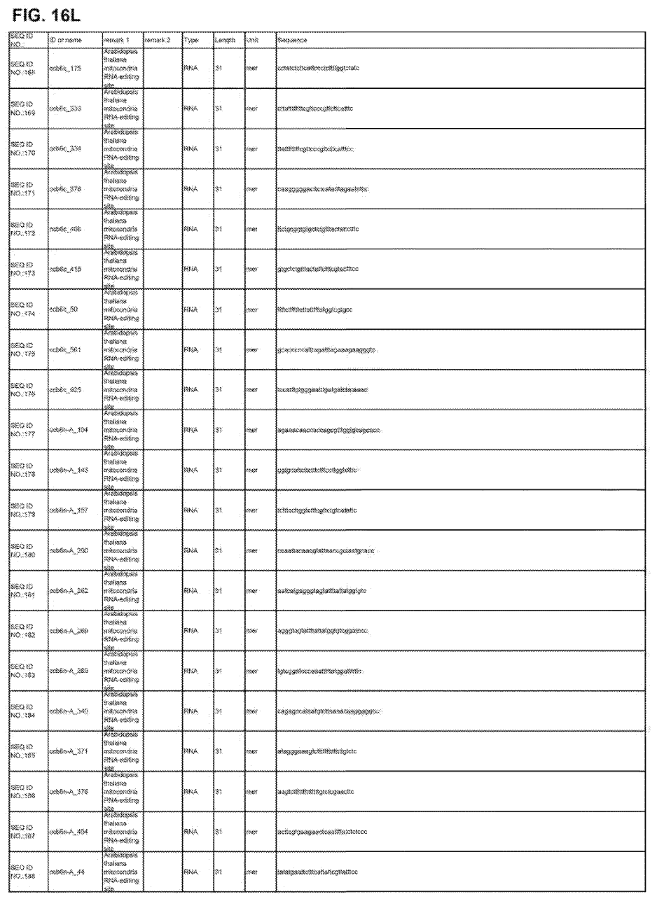

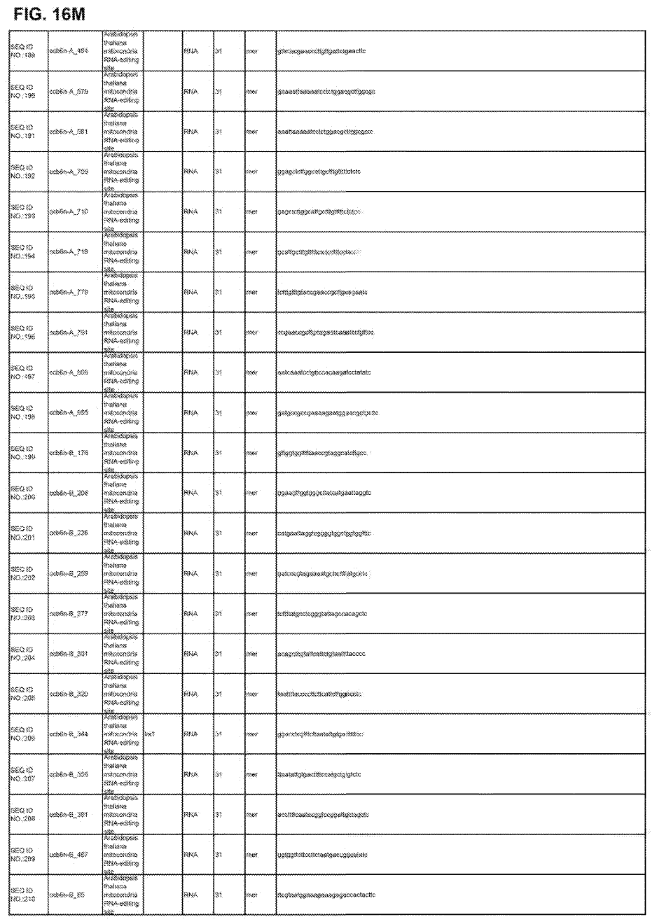

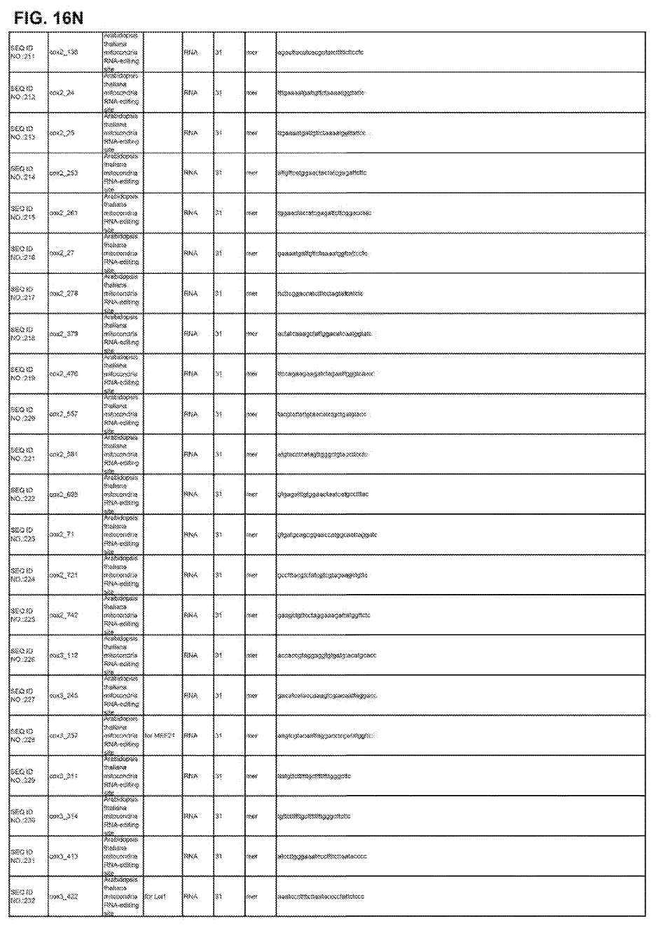

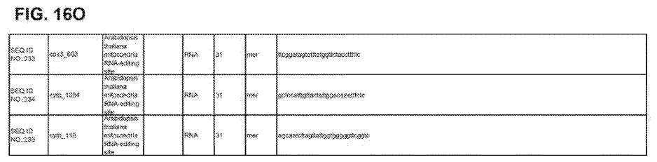

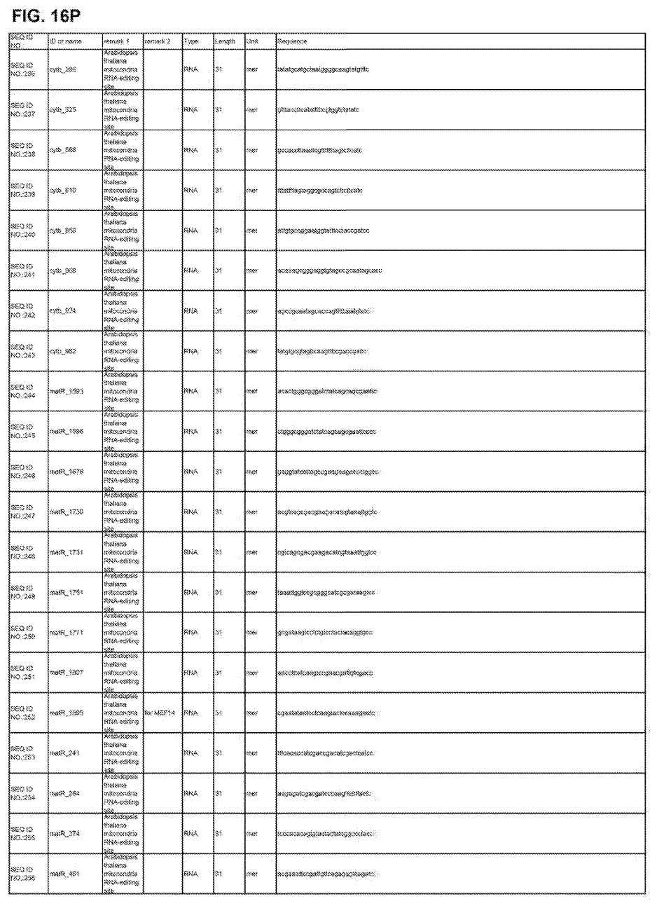

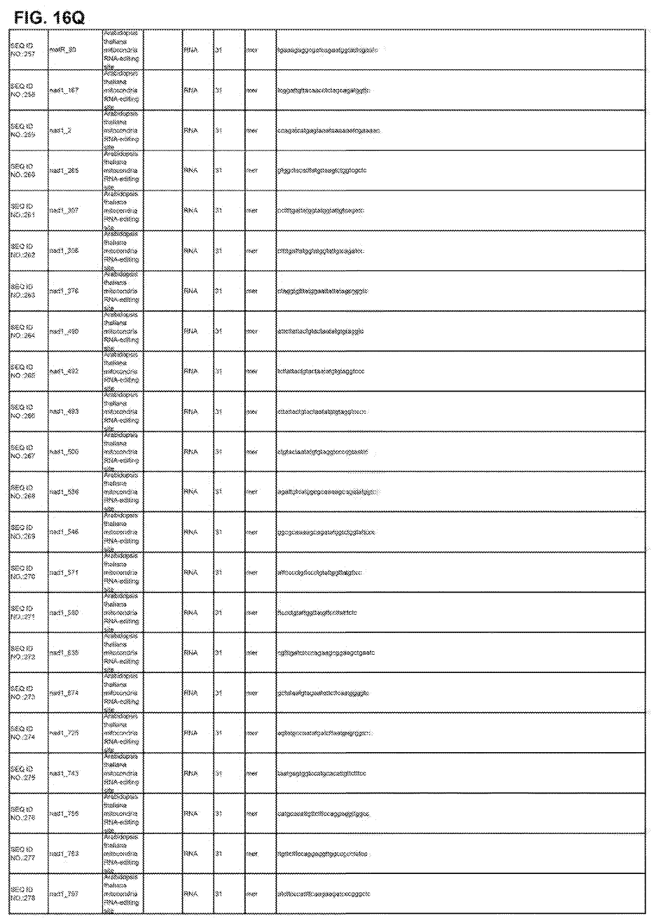

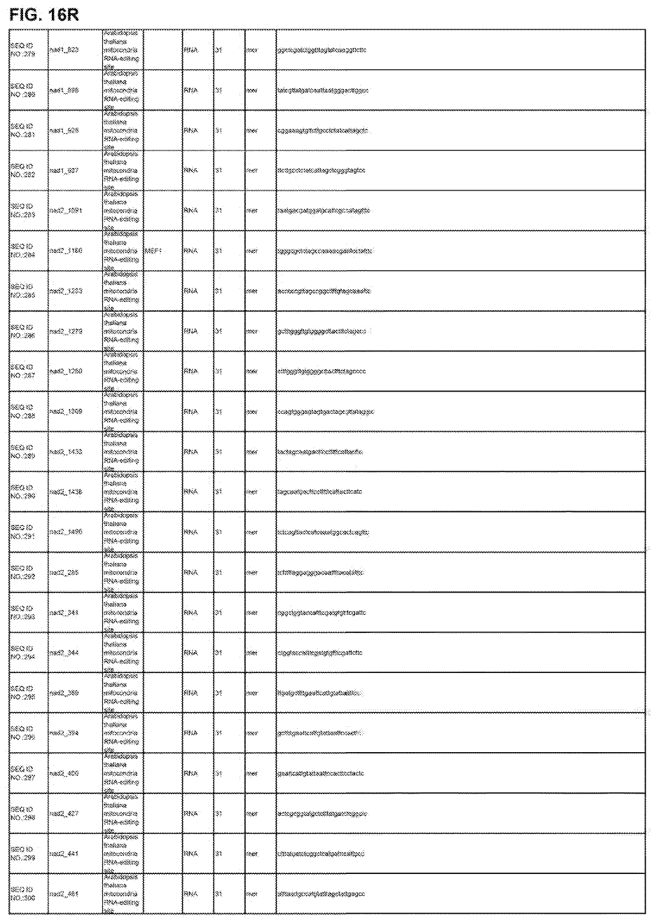

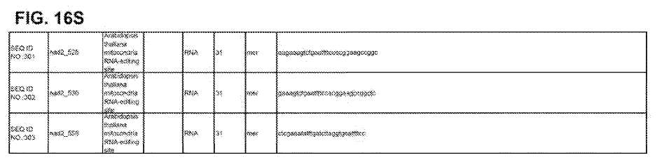

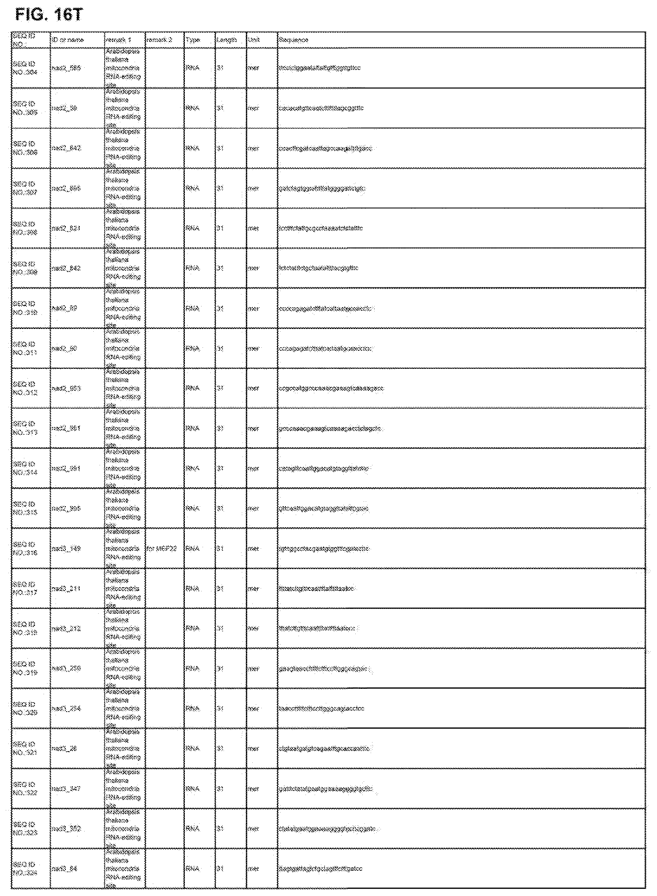

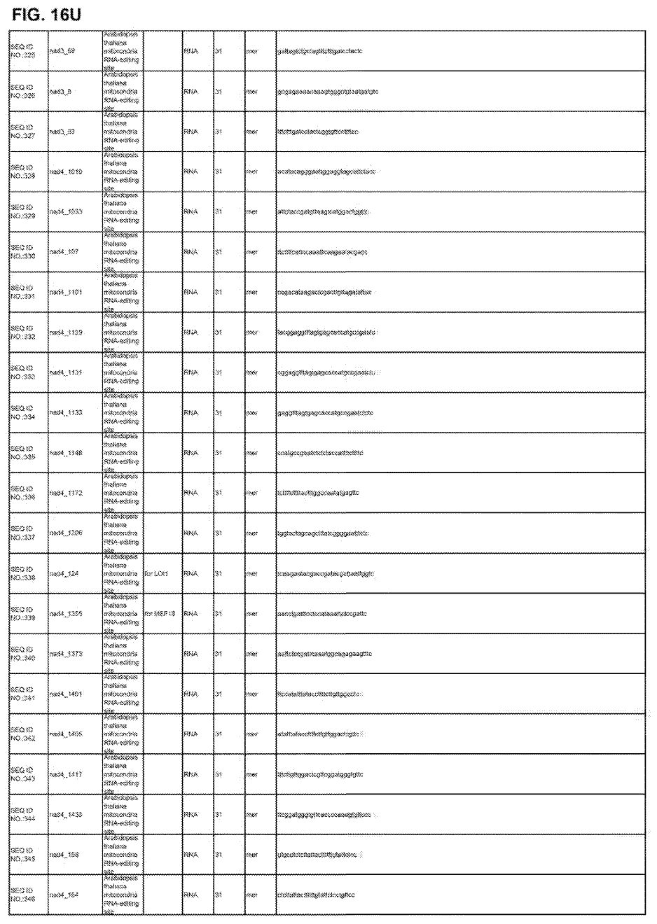

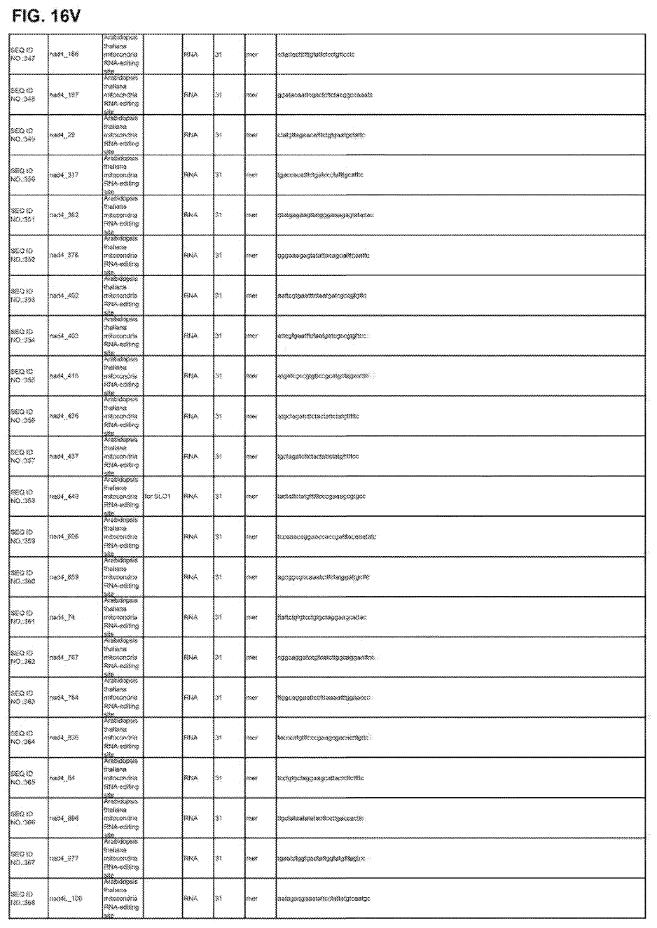

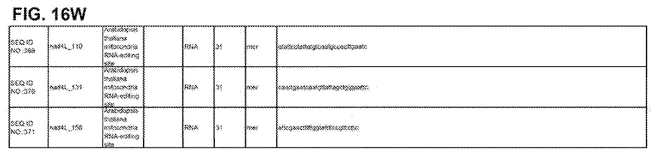

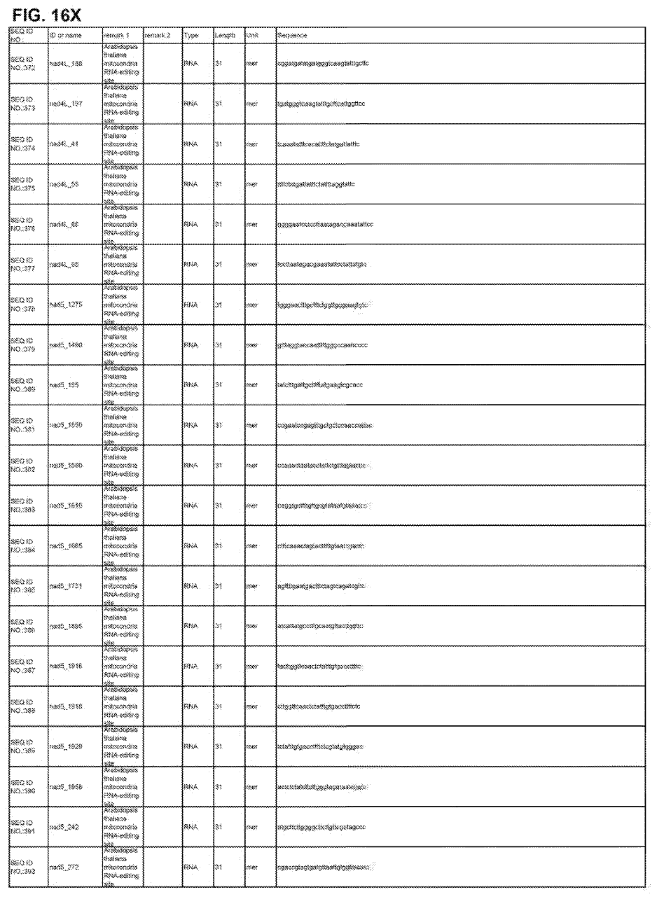

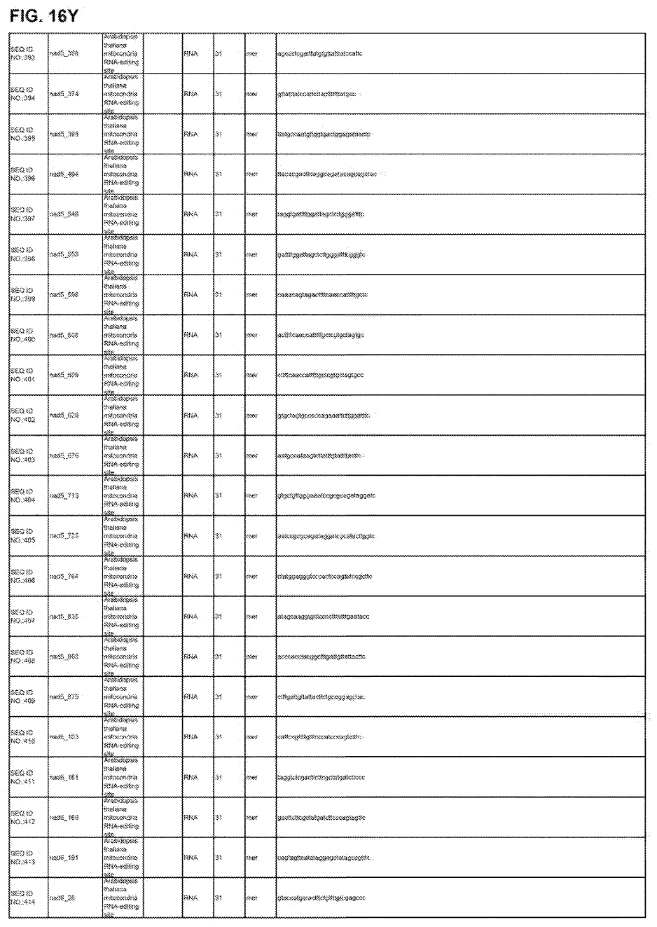

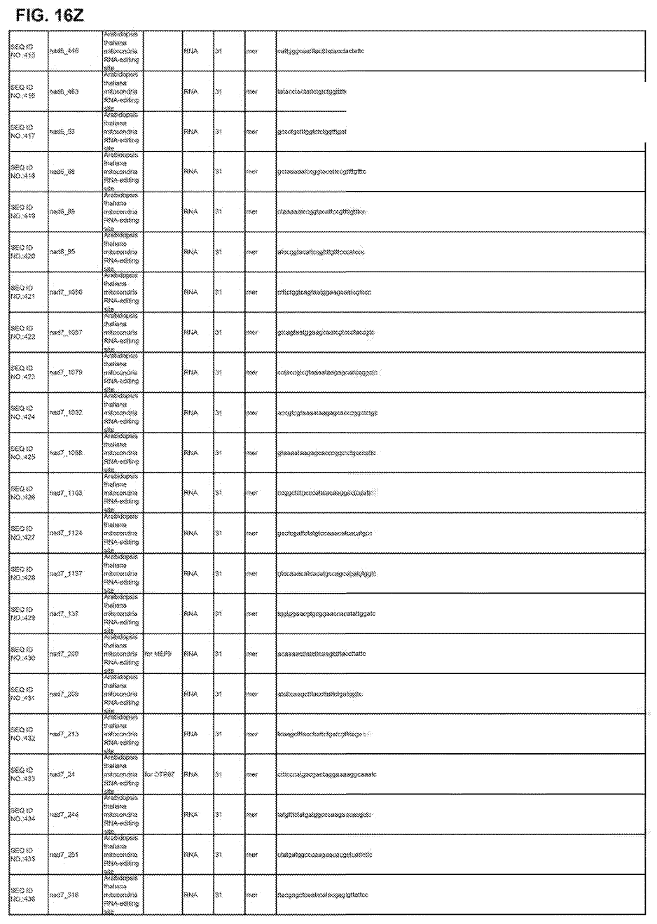

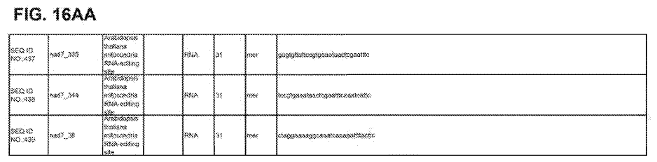

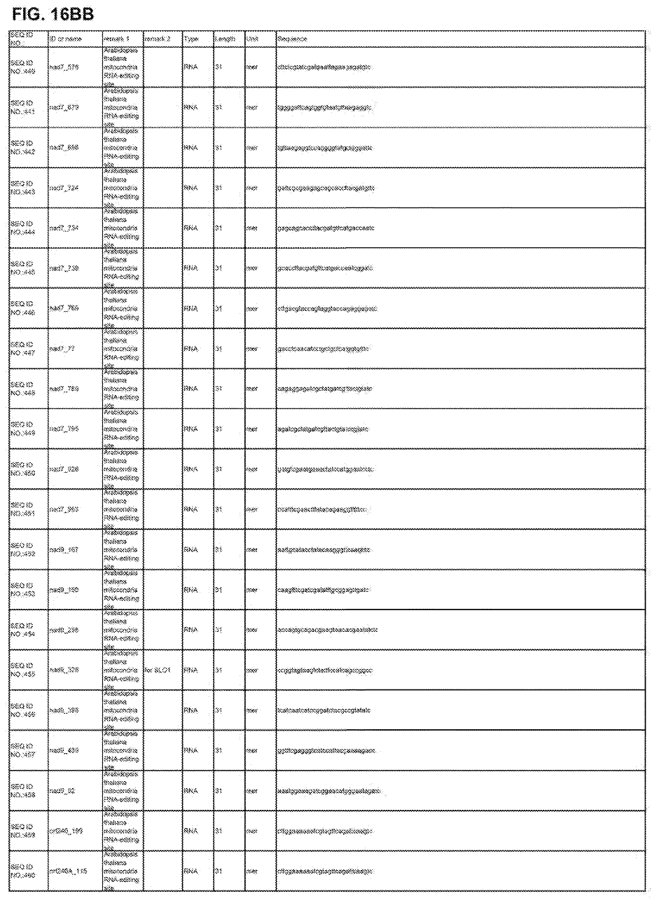

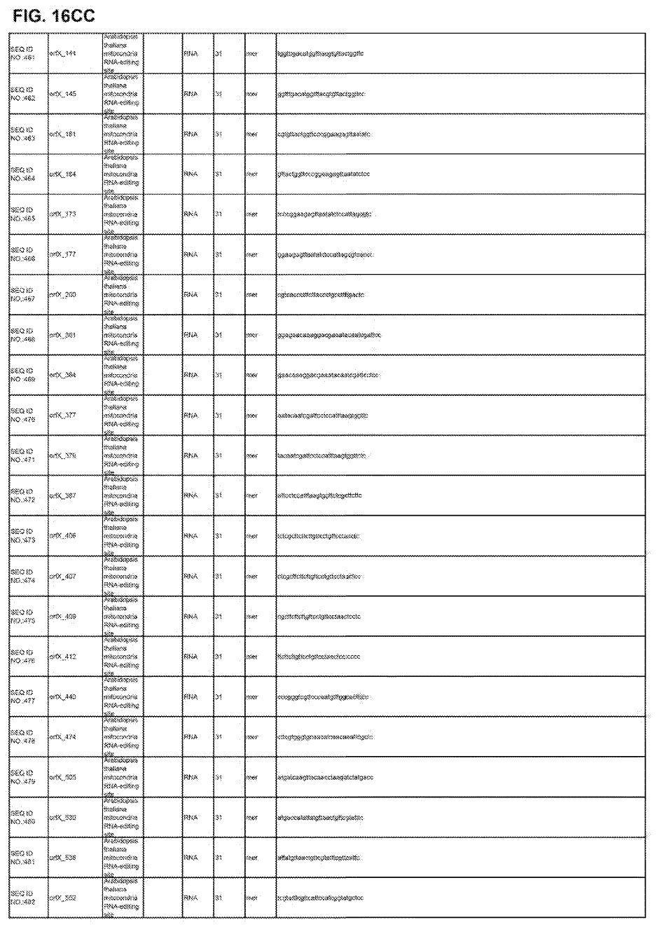

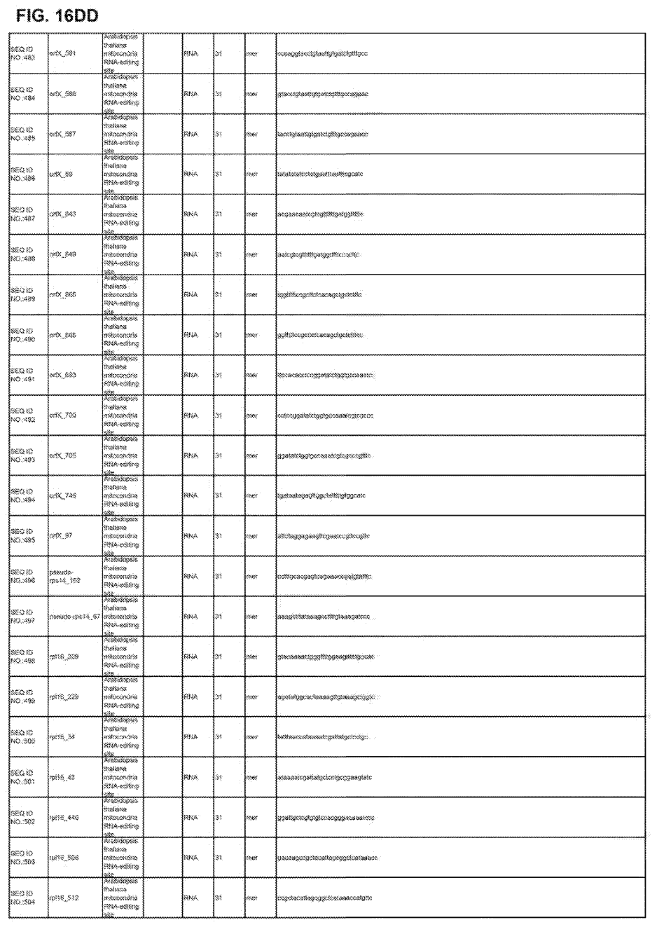

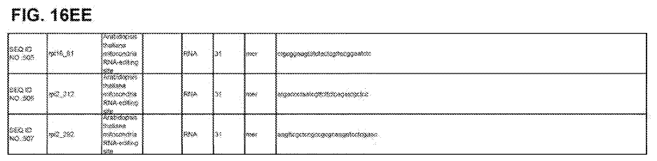

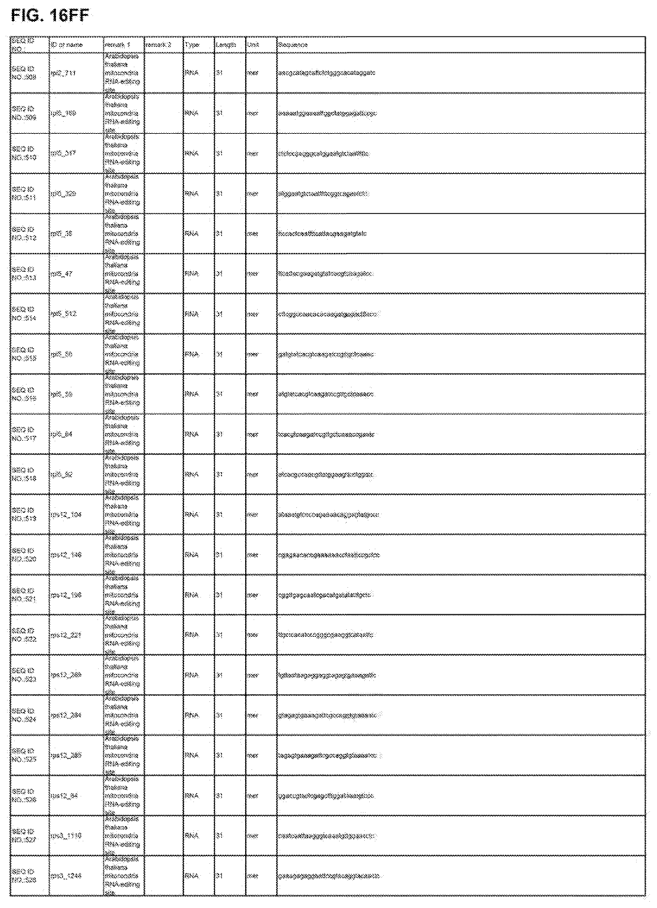

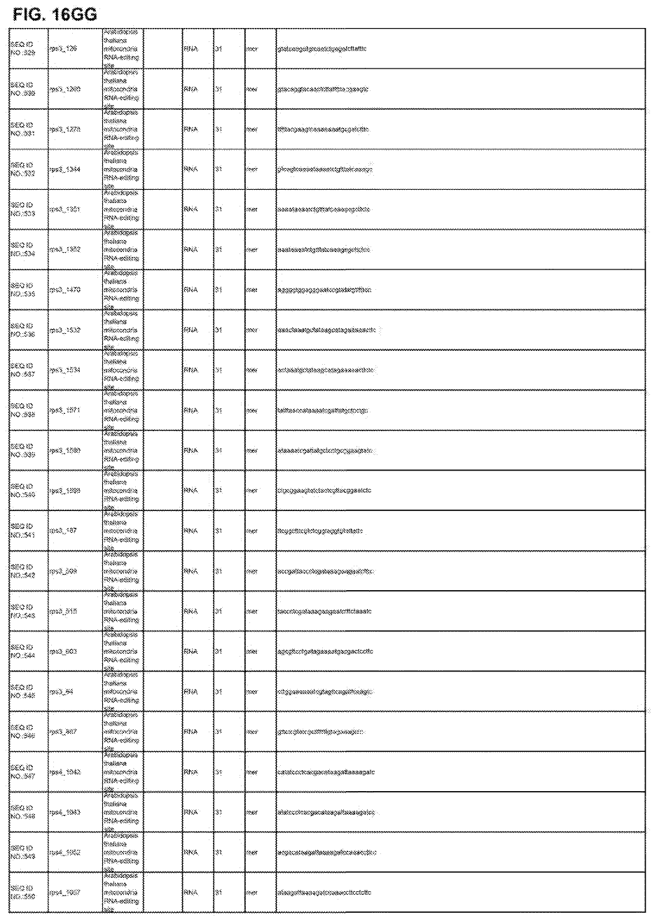

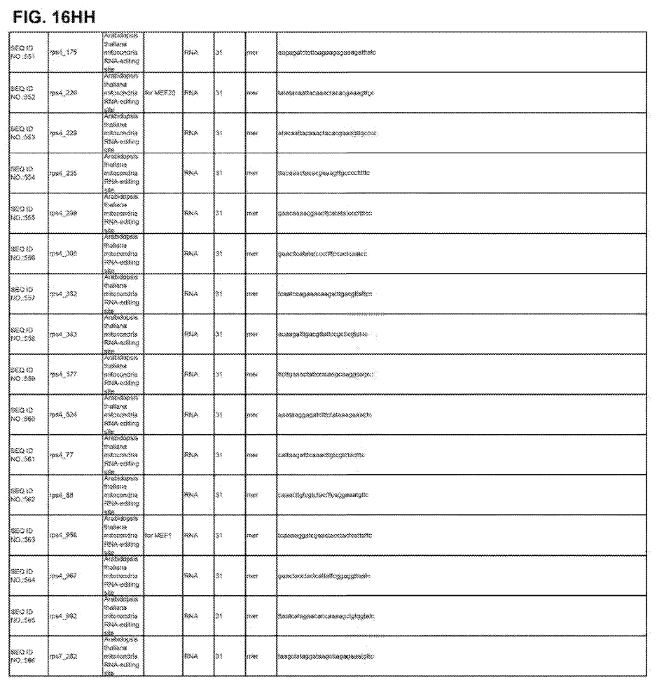

With reference to the information shown in FIG. 2, the PPR proteins of Arabidopsis thaliana involved in RNA editing so far analyzed (SEQ ID NOS: 2 to 24, see FIGS. 16A-16HH) were collected from the Arabidopsis thaliana genome information database (MATDB: http://mips.gsf.de/proj/thal/db/index.html), and sequences around RNA-editing sites that serve as a target (SEQ ID NOS: 48, 50, 53, 55, 57, 59, 60, 61, 62, 63, 64, 65, 68, 69, 70, 71, 73, 74, 76, 78, 80, 122, 206, 228, 232, 252, 284, 316, 338, 339, 358, 430, 433, 455, 552 and 563 (see FIGS. 16A-16HH)) were collected from the RNA-editing database (http://biologia.unical.it/py_script/overview.html). As the RNA sequences, those of 31 nucleotides upstream from the editable C (cytosine) residue including that C were collected. All the collected proteins and RNA-editing sites corresponding to the proteins are shown in FIG. 2.

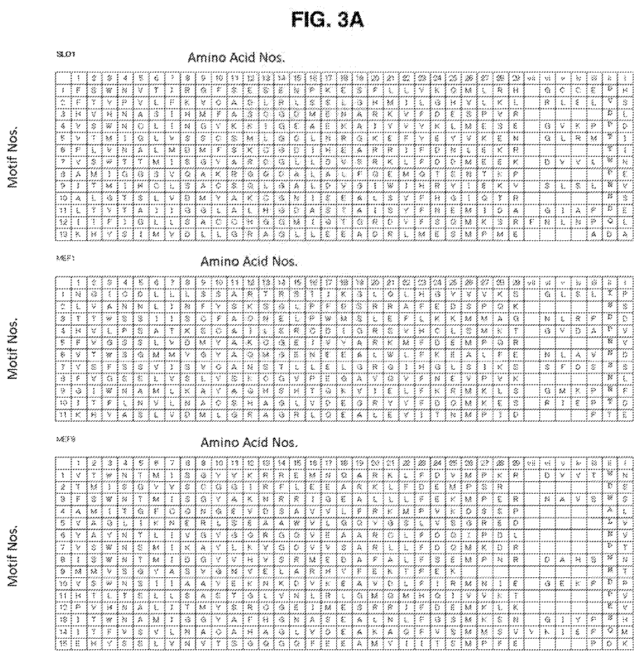

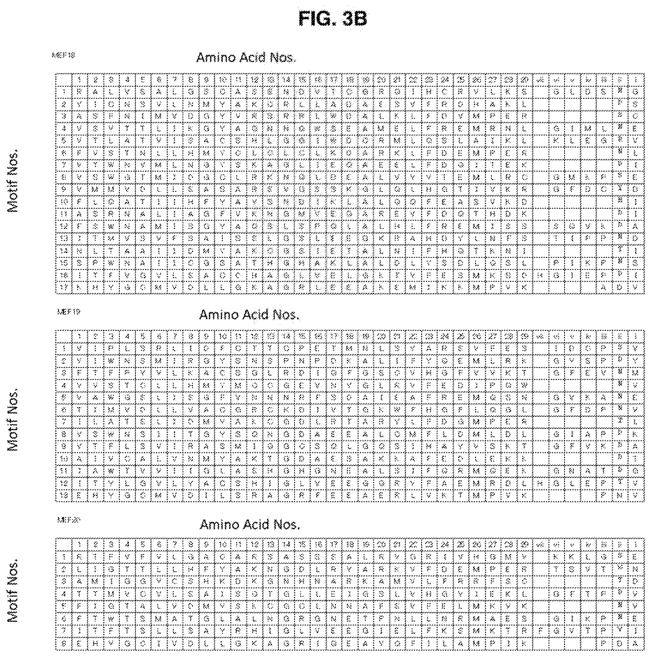

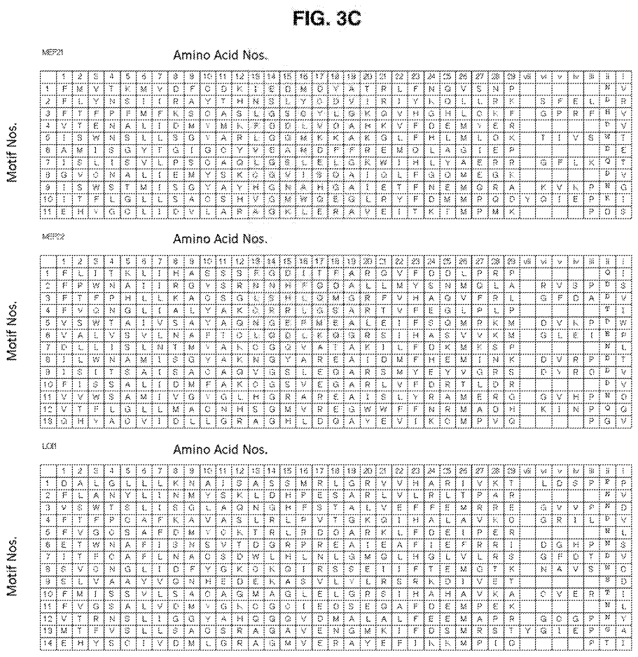

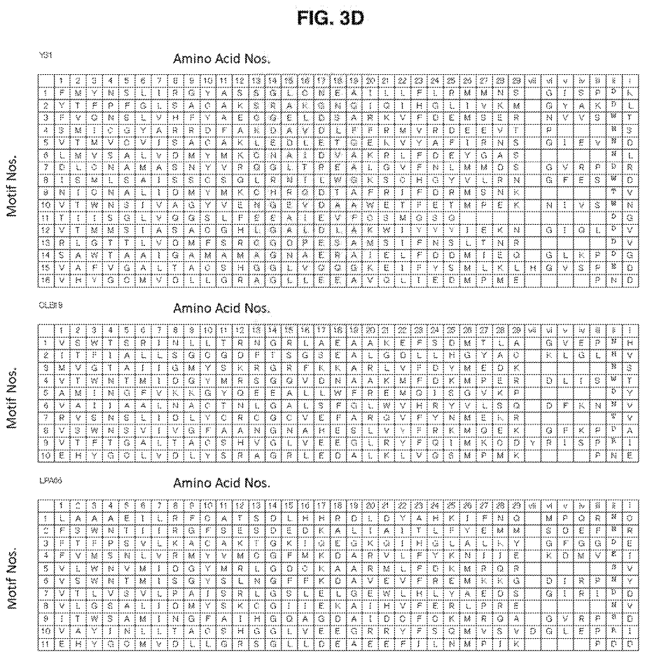

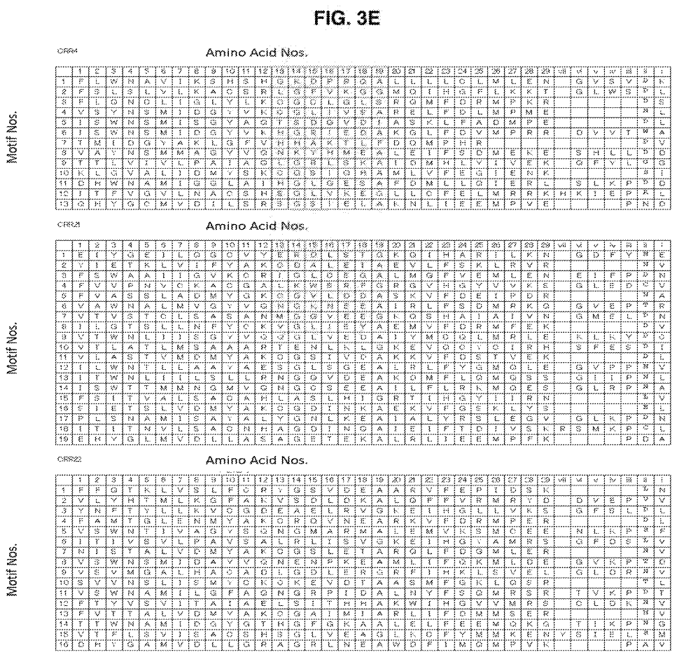

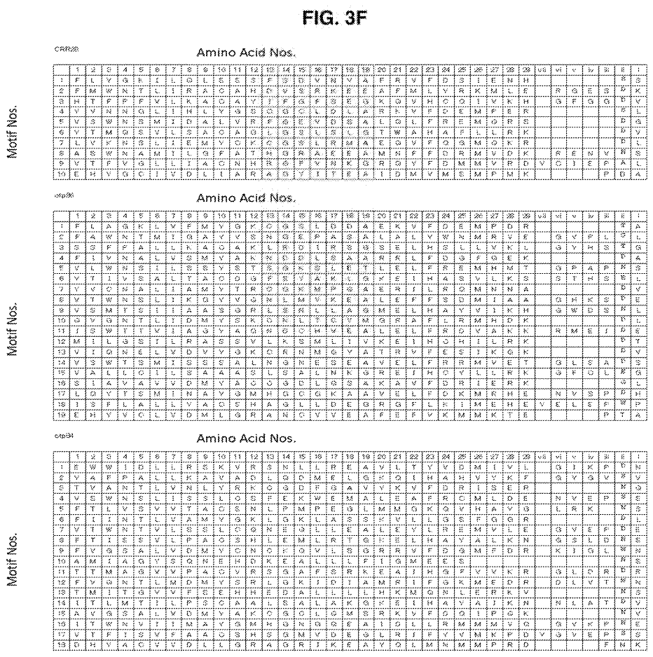

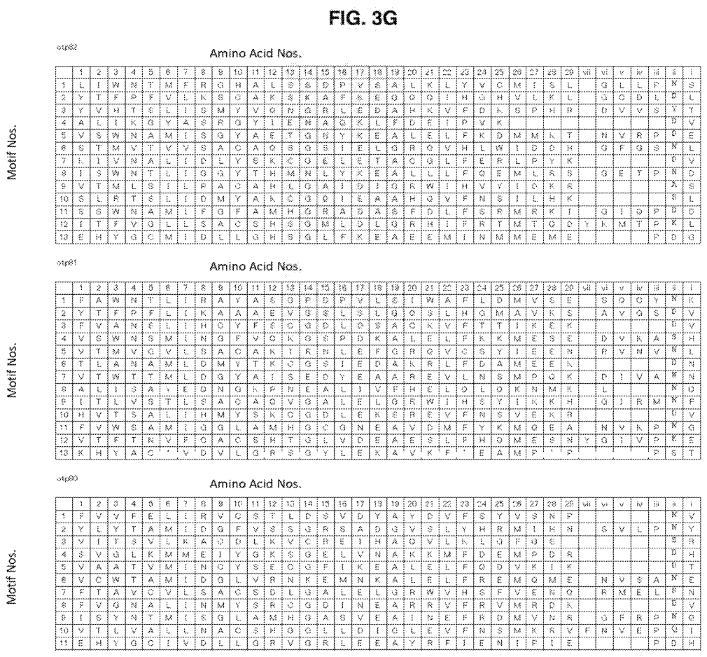

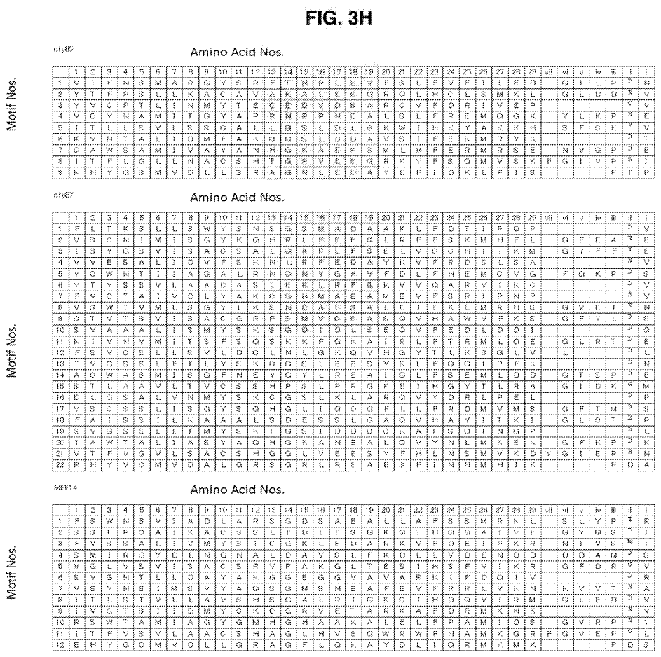

To the PPR motif structures in the proteins, the amino acid numbers defined in the present invention, as well as the information of the Uniprot database (http://www.uniprot.org/) are imparted. The PPR motifs contained in 24 of the Arabidopsis thalianas PPR proteins (SEQ ID NOS: 2 to 25, see FIGS. 16A-16HH) used for the experiments and amino acid numbers thereof are shown in FIGS. 3A-3H.

### Example 2: Identification of Amino Acids that Impart Binding Base Selectivity

The researches so far elucidated that the PPR proteins involved in RNA editing have a motif having a specific conserved amino acid sequence on the C-terminus side (E, E+ and DYW motifs, provided that DYW motif often does not exist). It has been suggested that more than ten amino acids in the E+ motif are required for the conversion from C (cytosine) to U (uracil), not for the selective binding to RNA (Reference 3). Further, it has also suggested in the past non-patent paper that the information required for recognition of the editable C is included in the 20 upstream nucleotides and 5 downstream nucleotides thereof. That is, it can be predicted that a plurality of PPR motifs in the PPR protein recognize “somewhere” of the upstream sequence of the editable C, and the E+ motif locates near the editable C. Furthermore, there is considered a possibility that specific amino acids in the PPR motif may recognize the RNA residue of the upstream sequence to which they bind (FIG. 4A).

This possibility was verified by using the 24 RNA-editing PPR proteins of Arabidopsis thaliana and target RNA sequences thereof described in Example 1. First, all the PPR motifs of the PPR protein were aligned with the corresponding RNA residues by arranging the last PPR motif in the protein at the first nucleotide from the editable C with 1-motif to 1-nucleotide correspondence in linear contiguity (FIG. 4A, alignment P1). Then, the RNA sequence was moved toward the right, 1 nucleotide at a time, to obtain the alignments P2 to P6. In the data set for each of these alignments P1 to P6, the information on the RNA residues corresponding to the PPR motifs was collected.

For a PPR protein that works for a single editing site, a score of 1 was given to each occurrence of the RNA nucleotide (A, U, G or C). For PPR proteins that work for 2 and 3 editing sites, scores of 0.5 and 0.3 were given to each occurrence of the RNA nucleotide, respectively. Then, the sets of PPR motifs and nucleotides were sorted according to types of amino acids for each of the amino acid numbers in the PPR motifs. It can generally be predicted that amino acids and RNA residues randomly appear for the types thereof (high-randomness or high-entropy) (an example is shown in the upper graph on the right side in FIG. 4A). However, if an amino acid of a specific position has binding RNA base selecting capacity, it is predicted that the corresponding RNA base is converged to one kind or limited kinds of them in correct alignments (P1 to P6 mentioned above) (low randomness or low entropy, an example is shown in the lower graph on the right side in FIG. 4A).

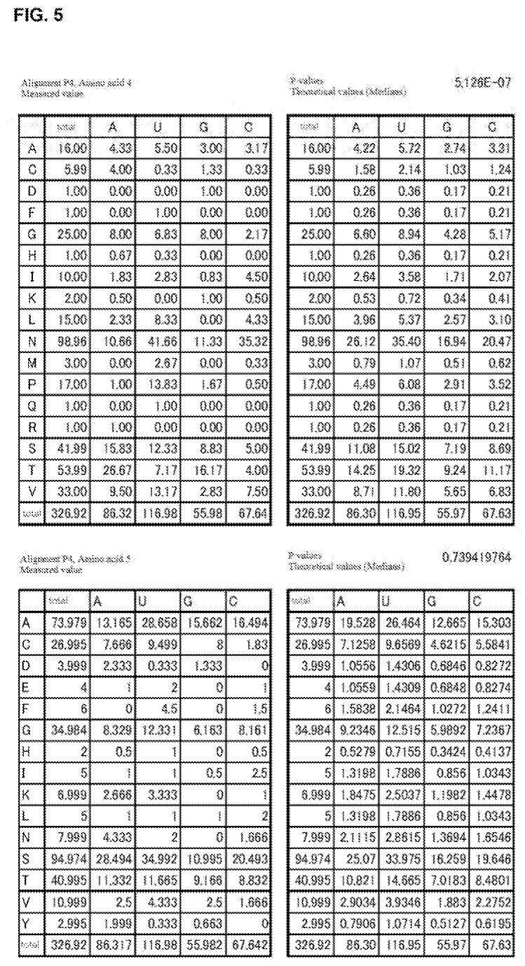

The aforementioned low randomness was calculated for all the amino acid numbers of the PPR motifs for the data sets of the alignments P1 to P6 created above. The low randomness was calculated by the chi square test based on a theoretical value (average of occurrence frequencies of all the nucleotides) (examples are shown in FIG. 5).

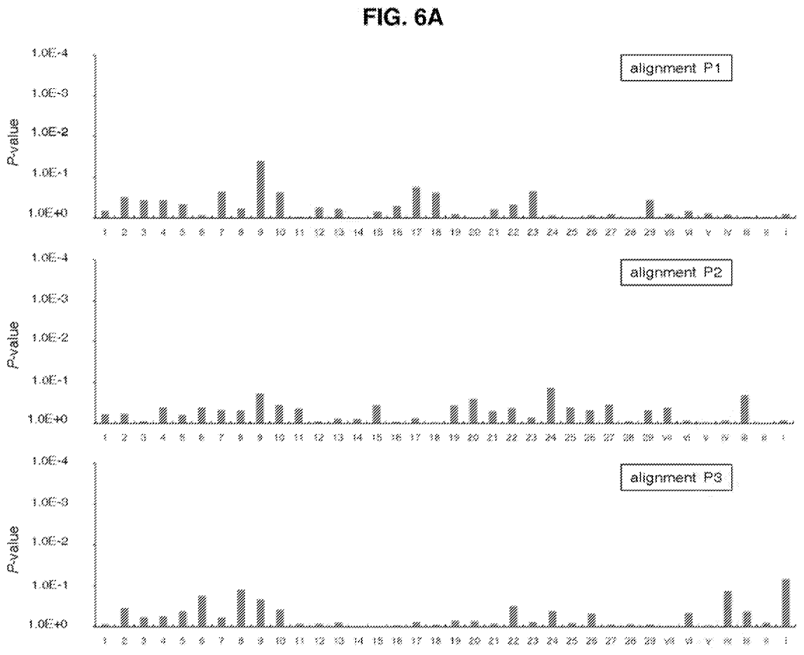

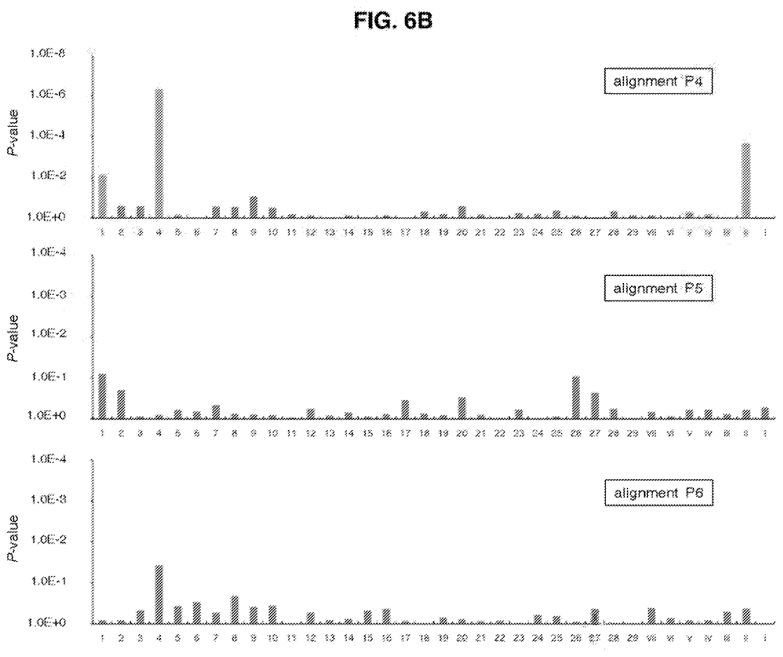

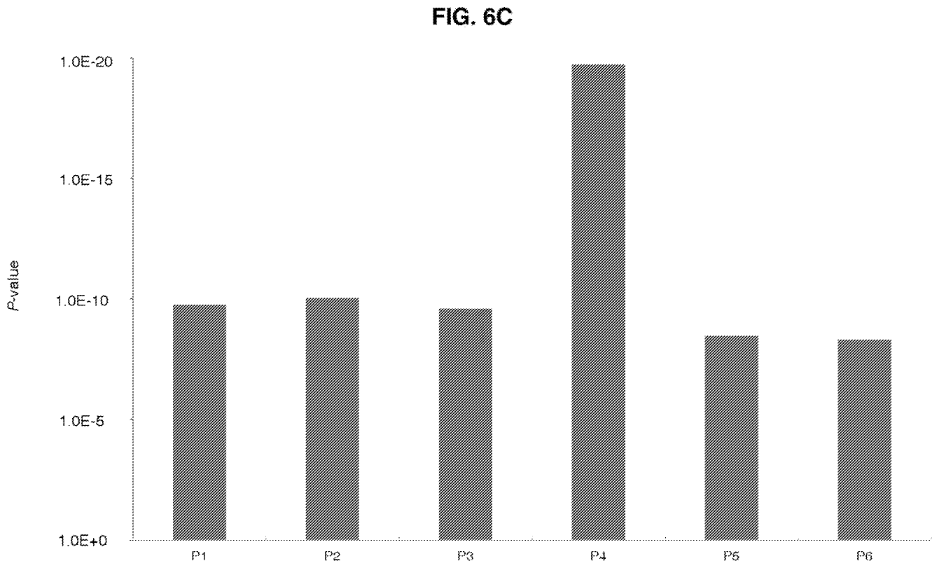

As a result, for amino acids 1, 4 and “ii” (−2) in alignment P4, it was determined that the significance value P is smaller than 0.01 (probability lower than 1%) (FIG. 4B). That is, it was revealed that the last PPR motif in the RNA-editing PPR protein is arranged at the base 4 nucleotides before the editable C, and the three amino acids (1, 4, and “ii”) are responsible for the binding RNA base selection. Further, because any significant P value was not calculated for the alignments P3 and P5, it was revealed that there is no interference from the PPR motifs of both sides, i.e., one PPR motif recognizes one RNA residue, and the binding does not depends on the constitution of the motifs. For the other amino acids in alignment P4, and all the amino acids of the other alignments, any significant P value was not obtained (FIGS. 6A-6C). Further, the RNA bases were classified into those of purine (A and G) or pyrimidine (C and U) (RY), and the same calculation was performed. As a result, an extremely significant P value (P<0.01) was obtained only for amino acid 4 (FIG. 4C). This indicates that amino acid 4 mainly determines which one of purine and pyrimidine is the RNA base to be bound. The binding base specifying capacity of the RNA recognition amino acids in the PPR motif shown in FIG. 4C was analyzed in more detail. As a result, in addition to that amino acid 4 mainly distinguishes the type of the base to which it binds, purine or pyrimidine (RY), it was found that the amino acid “ii” (−2) works to distinguish the form of the base, amino form (A and C) or keto form (G and U) (MK, FIG. 4D).

Combinations of the three amino acids (1, 4, and “ii”) used 3 times or more were defined as triPPR codes among the RNA recognition codes of the PPR motifs, and P value was calculated for each of them to calculate the binding RNA base specifying capacity thereof. A part of the identified triPPR codes are shown in FIG. 4E.

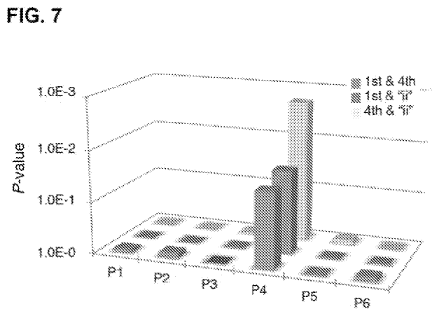

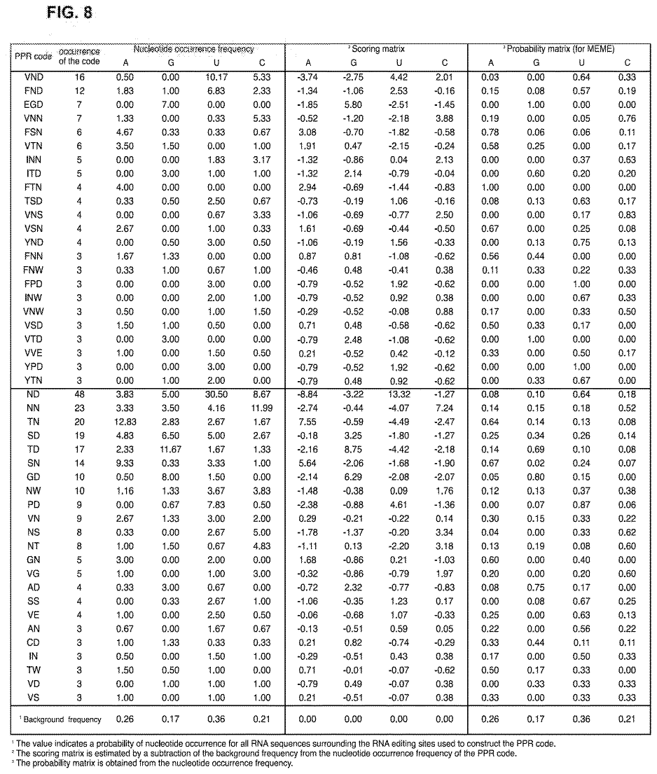

Since the amino acids of the three positions were extremely diverse, the binding RNA base specifying capacity was calculated for two amino acids (1 and 4, 1 and “ii”, or 4 and “ii”). As a result, a remarkable P value was calculated for the combination of amino acids 4 and “ii” (FIG. 7). Therefore, combinations of amino acids 4 and “ii” used 3 times or more were defined as diPPR codes among the RNA recognition codes of the PPR motifs. The identified triPPR codes and diPPR codes are shown in FIG. 8.

### Example 3: Verification of Identified RNA Recognition Codes

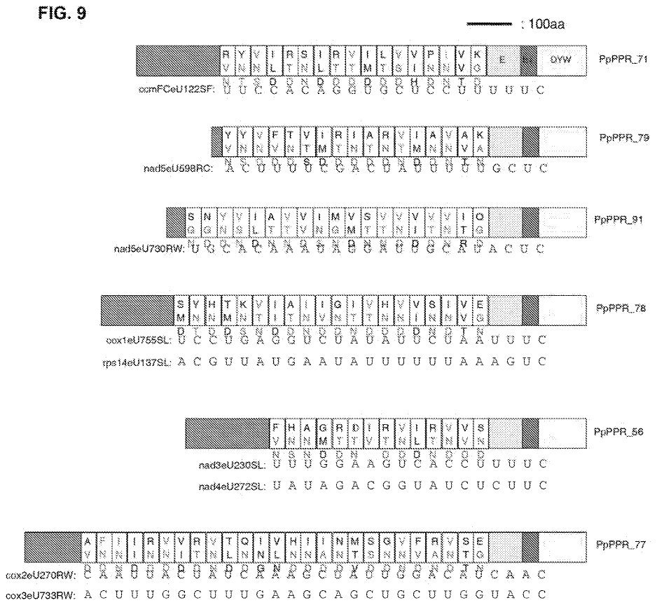

The RNA recognition codes for the PPR motifs identified by using the RNA-editing PPR proteins of Arabidopsis thaliana were verified. For the verification, the RNA-editing PPR proteins of Physcomitrella patens subsp. patens were used. It has already been elucidated that, in Physcomitrella patens subsp. patens (henceforth referred to as moss), RNA editing occurs at 13 sites in total (11 site in mitochondria, 2 sites in chloroplasts, SEQ ID NOS: 32 to 44 (see FIGS. 16A-16HH)). Further, it has also been elucidated that 6 PPR proteins (PpPPR_56, 71, 77, 78, 79, and 91) work for RNA editing at 9 sites, respectively. The proteins and corresponding RNA-editing sites are shown in FIG. 9.

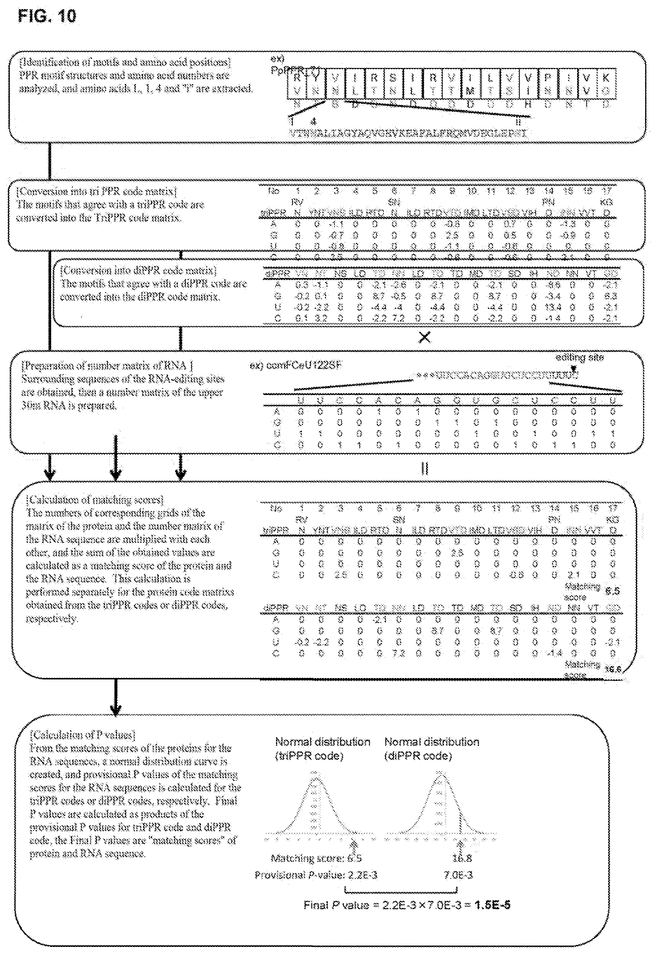

The verification was performed as shown in FIG. 10. First, the amino acid sequence information of the moss PPR proteins was obtained from a non-patent paper (SEQ ID NOS: 26 to 31, FIGS. 2, 9, and 16A-16HH), and the three amino acids (1, 4, and “ii”) were extracted from each PPR motif according to the PPR motif model defined as shown in FIGS. 1A-1C. When the combination of the extracted three amino acids agreed with any one of the triPPR codes identified from Arabidopsis thaliana, it was converted into a binding base scoring matrix represented by that code. Then, a PPR motif that could not be converted with any of the triPPR codes, but agreed with any one of the diPPR codes was converted into the binding nucleotide scoring matrix of diPPR code. In parallel, surrounding sequences of the RNA-editing sites (31-mer sequences having the editable C at the 3′ end) were obtained from a non-patent paper (SEQ ID NOS: 32 to 44, FIGS. 2, 9 and 16), and converted into such a number matrix of the RNA sequence as shown in FIG. 10. The numbers of corresponding grids of the binding base scoring matrix of the protein and the number matrix of the RNA sequence were multiplied with each other, so as not to contradict to the above-mentioned alignment P4 (the last PPR motif corresponds to the base 4 nucleotides before the editable C), and the sum of the obtained values was calculated as a matching score of the protein and the RNA sequence. This calculation was performed for the triPPR codes, diPPR codes, and the PPR binding base scoring matrixes (PPR scoring matrixes) thereof.

For one kind of protein, this calculation was performed for all the RNA-editing sites of the moss (13 sites). Further, the same calculation was also performed for 34 RNA sequences of the RNA-editing sites of Arabidopsis thaliana chloroplast (FIG. 16, SEQ ID NOS: 45 to 78) as reference sequences of RNA-editing site surrounding sequences.

Then, from the matching scores of the proteins for the RNA sequences, a normal distribution curve was created, and provisional P values of the matching scores for the RNA sequences were calculated for the triPPR codes and diPPR codes, respectively.

Final P values (matching scores of protein and RNA sequence) were calculated as products of the provisional P values for triPPR code and diPPR code.

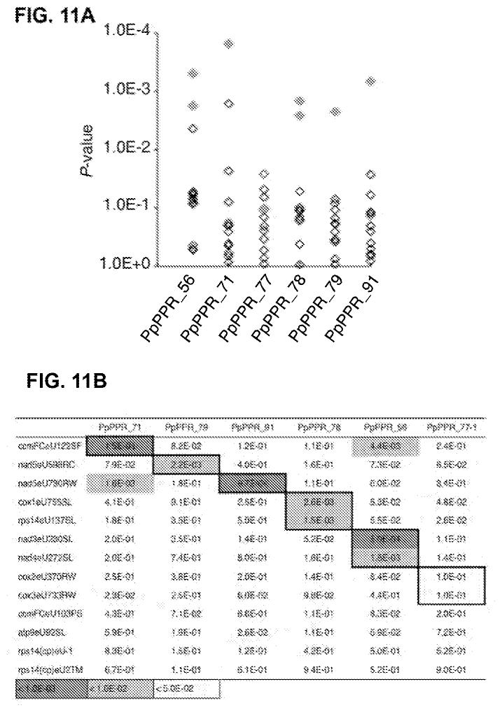

The matching scores of the moss PPR proteins and 13 moss RNA-editing sites are shown in FIGS. 11A and 11B. As a result of the analysis, 6 kinds of the proteins were computationally specified for the correct RNA-editing sites out of the 7 kinds of the proteins. That is, this analysis revealed that all the information for the binding RNA base specification performed by the PPR motif is contained in the three amino acids (1, 4, and “ii”). In other words, it was revealed that a PPR protein that binds to an intended RNA sequence can be searched for by referring to the information on the combinations of the two or three amino acids shown in FIG. 8 (triPPR and diPPR codes). At the same time, it was also shown that an artificial protein that binds to an intended RNA sequence can be synthesized by using or binding a PPR motif having such amino acid information.

### Example 4: Identification of Target Molecules of Unanalyzed RNA-Editing PPR Proteins

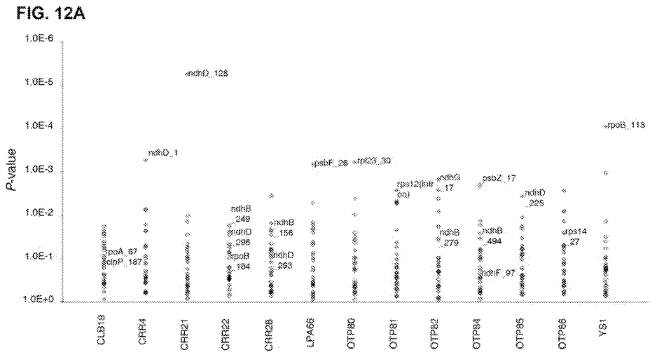

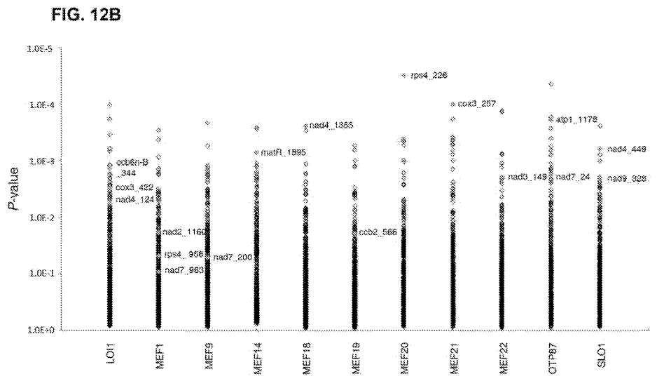

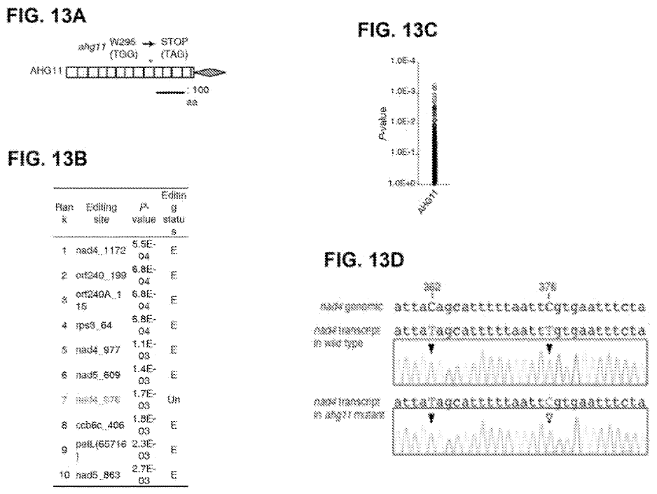

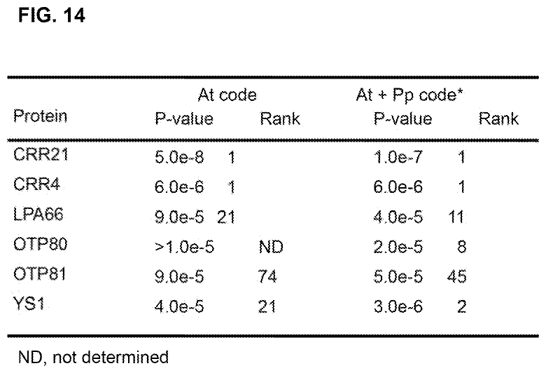

Then, analysis was performed by using Arabidopsis thaliana, which has a larger number of RNA-editing sites compared with the moss (34 sites in chloroplastic genome (SEQ ID NOS: 45 to 78 (see FIGS. 16A-16HH)), and 488 sites in mitochondrial genome (SEQ ID NOS: 79 to 566 (see FIGS. 16A-16HH)), see FIGS. 6A-6C). In order to verify prediction accuracy, RNA-editing sites of 24 kinds of PPR proteins used for the code extraction were predicted. As a result, for the chloroplast-localized PPR proteins, at least one correct RNA-editing site was predicted with the highest P value for 10 kinds of proteins out of 13 kinds of the proteins. For mitochondria-localized PPR proteins, a correct RNA-editing site was predicted with a value within top 20 thereof for 8 kinds of proteins out of 11 kinds of the proteins (FIGS. 12A-12B). On the basis of the results of this verification of prediction accuracy, target RNA-editing sites of the PPR proteins of which function was unknown were predicted. An AHG11 mutant is a mutant having anomaly in the abscisic acid pathway, and the proteins encoded by the genes thereof (ahg11, at2g44880) have a typical RNA-editing PPR protein-like motif structure (FIG. 13, SEQ ID NO: 1 (see FIGS. 16A-16HH)). RNA-editing sites were predicted, and 405 sites for mitochondria and 30 sites for chloroplasts including those of values within the top 20 thereof were experimentally verified. As a result, it was revealed that only the RNA editing of mitochondria nad4_376 predicted with the 7th highest P value had anomaly in the mutant (FIGS. 13A-13D).

Then, it was attempted to identify target RNA sequences in the total genomes of the organelles i.e., a data set of about 3×10⁵ RNA sequences. For this analysis, the probability matrix of PPR codes shown in FIG. 8 was used. Further, for the motifs having a combination of amino acids not agreeing with any of the diPPR and triPPR codes, background frequency was applied. The probability matrixes of the produced proteins were subjected to the FIMO analysis in MEME suite (http://meme.nbcr.net/meme4_6_1/fimo-intro.html) together with the chloroplast total nucleotide sequence of Arabidopsis thaliana (AP000423).

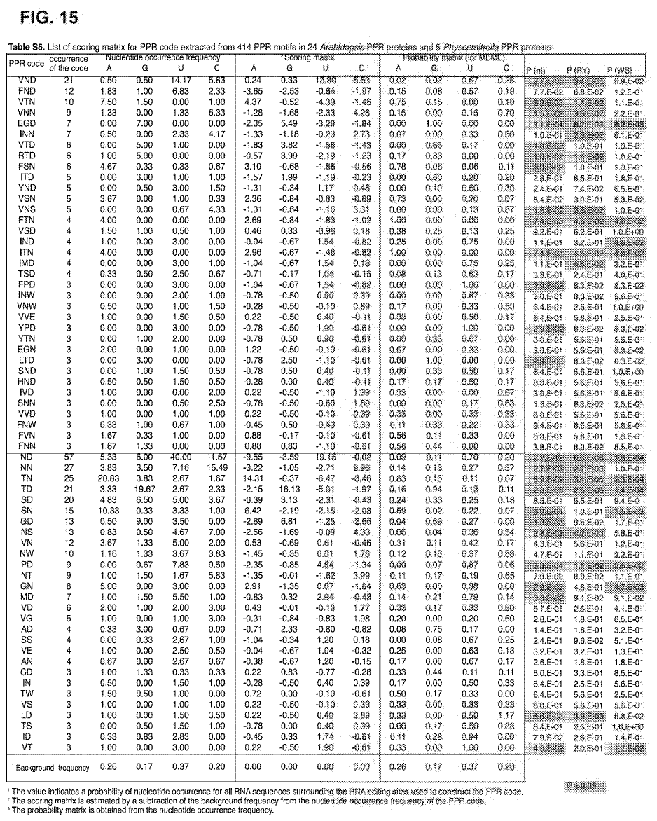

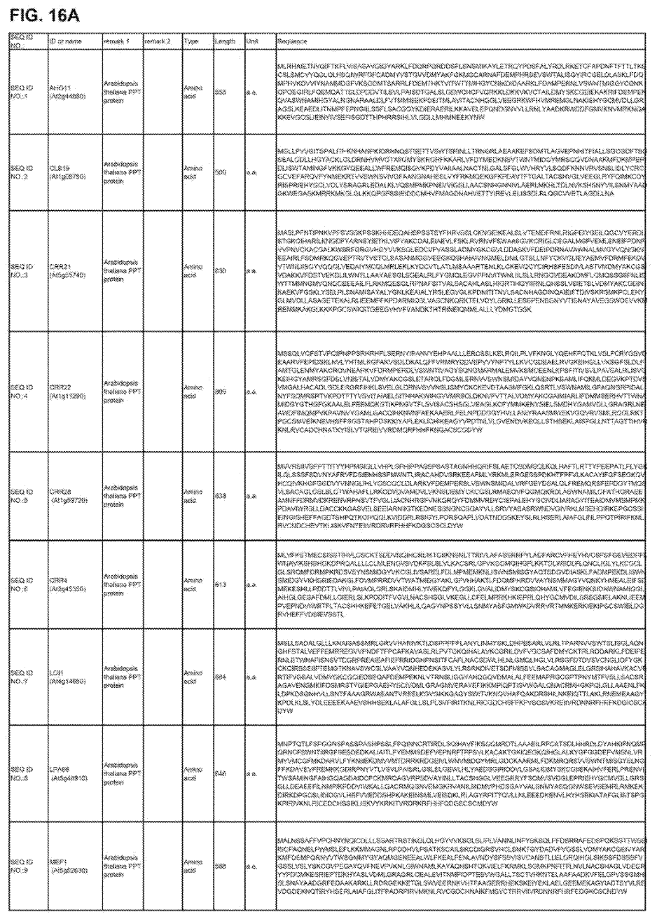

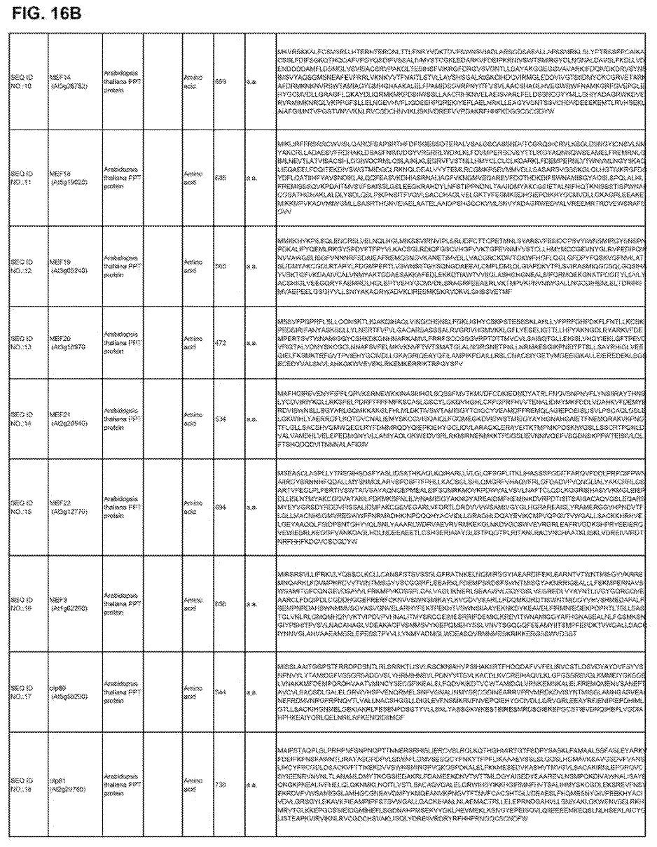

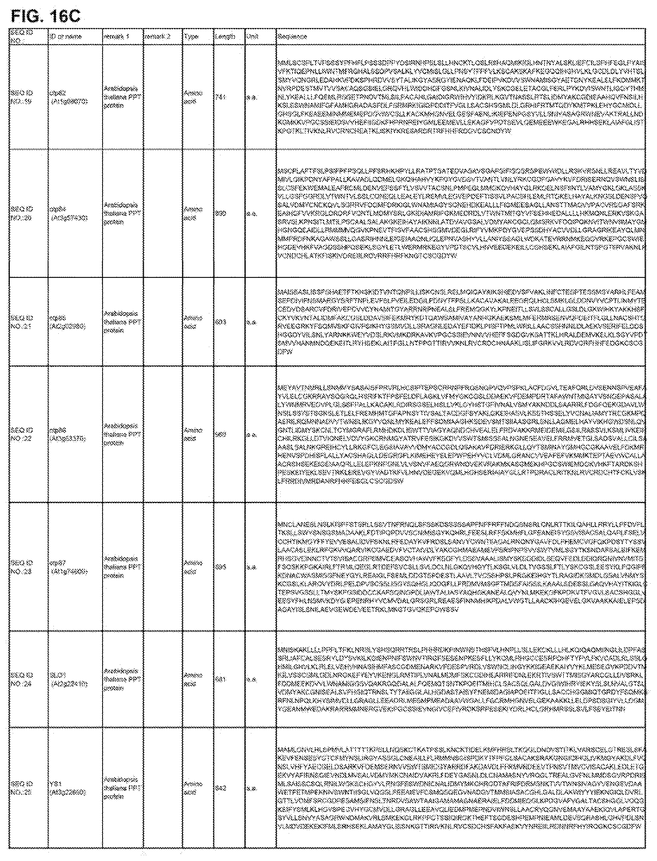

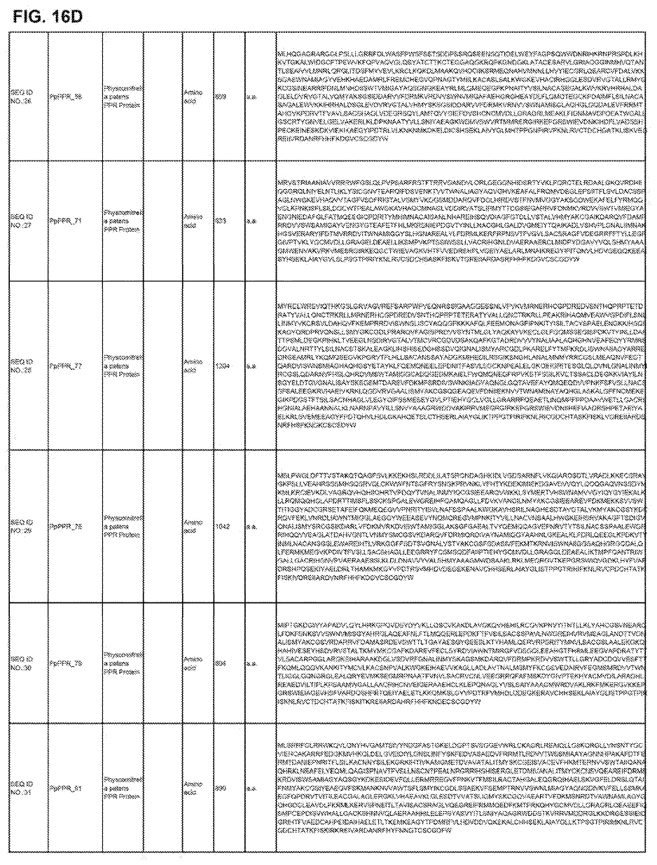

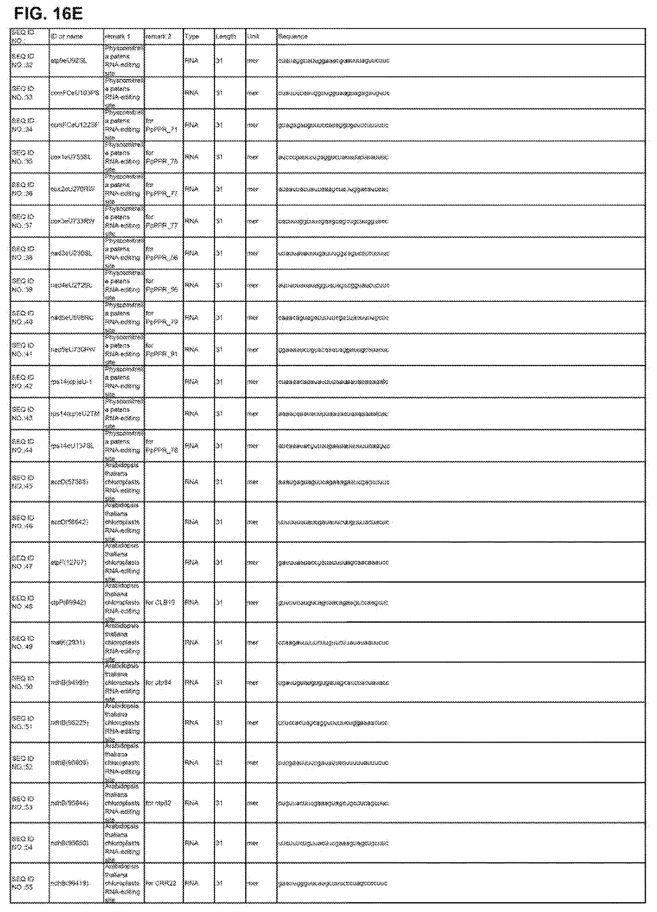

As a result, for CRR4 and CRR21, target RNA sequences thereof could be correctly predicted. Further, the codes were improved by extracting the PPR codes also from the moss PPR proteins (FIG. 15). As a result, the prediction accuracy was markedly improved for several proteins.

These results indicate that one correct target sequence can be identified from RNA sequences of several hundreds of thousands patterns by using the identified PPR codes. Conversely, by searching for a PPR motif having amino acids matching the code at the positions (1, 4, and “ii”), a protein that binds to the intended useful RNA sequence can be identified. Alternatively, it was shown that, by binding a PPR motif, an artificial RNA binding protein showing high sequence selectivity can be created. It will also be understood by those skilled in the art that, by obtaining a combination of amino acids at the concerned positions matching any of the PPR codes through introduction of mutation, intended RNA binding selectivity can be imparted. FIG. 15 shows evaluation of the binding RNA base selecting capacity of triPPR codes and diPPR codes based on the P values. It can be estimated that PPR codes that showed a significant P value (P<0.05) have high binding RNA base selecting capacity.

### Example 5: Prediction of Target RNA Sequence of Radish Rf

Then, on the basis of the findings obtained by the present invention, functions of the PPR proteins that work as a fertility restoration factor for cytoplasmic male sterility were determined (Examples 5 to 9).

The cytoplasmic male sterility (CMS) is a characteristic that the male gamete comes to no longer normally function due to a mutation in a cytoplasmic genome, especially a mitochondrial genome. It is known that this characteristic is compensated by a fertility restoration gene (restorer of fertility, Rf), which often exists in the nucleus, and the male gamete is thereby made normal. This characteristic is used for the first filial hybrid breeding method, and is one of the agriculturally important characteristics. It is known that, in such a CMS-Rf system, the Rf gene codes for a PPR protein in many cases.

Sterility of the Ogura-type (synonym, Kosena-type) cytoplasm used in the first filial hybrid breeding method for radish or rapeseed originates in expression of the orf125 gene in a mitochondrial genome, and canceled by the presence of the nuclear-encoded orf687 gene, and the cytoplasm acquires fertility. The orf687 gene product is a PPR protein, and it is considered that it acts on RNA containing orf125 to inactivate the expression thereof, and the sterility is canceled as a result.

However, it has become clear from the past thremmatological analyses that amino acid polymorphism is observed for the orf687-like genes of various radish pedigrees, and that this amino acid polymorphism affects the function of the gene as a fertility restoration factor. However, any method for estimating functionality of a gene from the amino acid sequence encoded thereby has not been established.

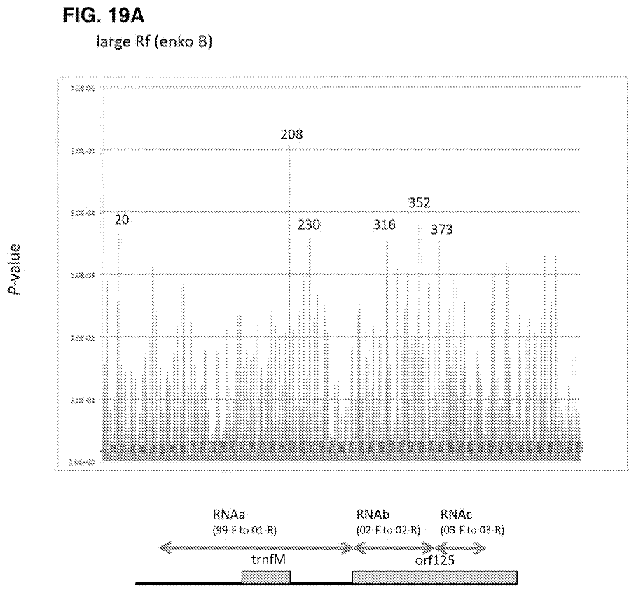

Therefore, a PPR motif was first specified in the amino acid sequence of the ORF687 protein of the radish variety Enko (named Enko B), which is known to function as a dominant Rf, amino acids responsible for the base specifying capacity (1, 4, and ii) were extracted from it, and converted into a PPR code, and then the target RNA sequence thereof was predicted for a transcription product containing the mitochondrial orf125 (FIGS. 19A-19B)

In parallel, three kinds of ORF687-like proteins, the ORF687 protein of the radish variety Enko (named Enko B), which is known to function as a dominant Rf, an ORF687-like protein that is similarly contained in Enko and well resembles the ORF687, but acts as a recessive gene (named Enko A), and a gene homologous to the Enko ORF687 existing in the genome of Kosena, which is a different radish variety (named Kosena B, recessive gene), are used as experimental materials, and the characteristics of them were biochemically analyzed.

(5-1) Preparation of the Genomic DNA from Radish

Radish was cultured on the Murashige and Skoog medium (containing 2% sucrose and 0.5% Gellangam) for three weeks. The green leaves (0.5 g) of the cultured plant were extracted with phenol/chloroform, and then ethanol was added to insolubilize DNA. The collected DNA was dissolved in 100 μl of the TE solution (10 mM Tris-HCl (pH 8.0), 1 mM EDTA), 10 units of RNase A (DNase-free, Takara Bio) was added to the mixture, and the reaction was allowed at 37 °C for 30 minutes. Then, the reaction mixture was extracted again with phenol/chloroform, and DNA was collected by ethanol precipitation. DNA was obtained in an amount of 10 μg.

### (5-2) Cloning of Genes Coding for ORF687-Like Proteins

By performing PCR using radish genomic DNA as the template, oligonucleotide primers, Enko_B-F primer and Enko B-R primer (SEQ ID NOS: 567 and 568, respectively), for Enko B, oligonucleotide primers, kosena_B-F primer and kosena_B-R primer (SEQ ID NOS: 569 and 570, respectively), for Kosena B, or oligonucleotide primers, Enko_A-F primer and Enko_A-R primer (SEQ ID NOS: 571 and 572, respectively), for Enko A, and KOD-FX (TOYOBO) as a DNA extension enzyme in 50 μl of a reaction mixture with 25 cycles of 95 °C for 30 seconds, 60 °C for 30 seconds, and 72 °C for 30 seconds, the genes were amplified, respectively.

The obtained DNA fragments were cloned by using the pBAD/Thio-TOPO vector (Invitrogen) according to the attached protocol. The DNA sequences were determined to confirm that the sequences were those homologous to the intended corresponding DNA sequences (Enko B (SEQ ID NO:573), Kosena B (SEQ ID NO: 574), Enko A (SEQ ID NO: 575)).

### (5-3) Preparation of Recombinant ORF687-Like Proteins

The Escherichia coli TOP10 strain (Invitrogen) was transformed with the plasmids obtained above. The Escherichia coli strain was cultured at 37 °C in 300 ml of the LB medium containing ampicillin at a concentration of 100 μg/ml (300 mL of the medium contained in a 1-L conical flask). When the turbidity of the culture medium in terms of the absorbance at a wavelength of 600 nm reached 0.5, L-arabinose as an inducer was added at a final concentration of 0.2%, and culture was further continued for 4 hours.

The cells were collected by centrifugation, then suspended in 200 ml of Buffer A (50 mM Tris-HCl (pH 8.0), 500 mM KCl, 2 mM imidazole, 10 mM MgCl₂, 0.5% Triton X100, 10% glycerol) containing 1 mg/ml of lysozyme, and disrupted by ultrasonication and freezing/thawing. The cell suspension was centrifuged at 15,000×g for 20 minutes, and then the supernatant was collected as a crude extract.

This crude extract was applied to a column filled with a nickel column resin (ProBond A, Invitrogen) equilibrated with Buffer A.

After the column was sufficiently washed with Buffer A containing 20 mM imidazole, column chromatography was performed with two-step concentration gradient, in which the objective protein was eluted with Buffer A containing 200 mM imidazole. The obtained proteins were fusion proteins comprising the amino acid sequence of SEQ ID NO: 576 (Enko B), SEQ ID NO: 577 (Kosena B), or SEQ ID NO: 578 (Enko A), the amino acid sequence of thioredoxin for enhancing solubility on the N terminus side, and a histidine tag sequence on the C-terminus side. Each purified fraction in a volume of 100 μl was dialyzed against 500 mL of Buffer E (20 mM Tris-HCl (pH 7.9), 60 mM KCl, 12.5 mM MgCl₂, 0.1 mM EDTA, 17% glycerol, 2 mM DTT), and then used as a purified sample.

### (5-4) Preparation of Substrate RNA

As the substrate RNA, three kinds of RNAs containing the sequence of a mitochondrial DNA of Ogura-type radish cytoplasm, RNAa, RNAb, and RNAc, were used.

The DNAs were amplified by PCR using oligonucleotide primers, A-F primer and A-R primer (SEQ ID NOS: 579 and 580, respectively), for RNAa, oligonucleotide primers, B-F primer and B-R primer (SEQ ID NOS: 581 and 582 respectively), for RNAb, or oligonucleotide primers, C-F primer and C-R primer (SEQ ID NOS: 583 and 584, respectively), for RNAc, and KODFX (TOYOBO) as a DNA extension enzyme, in 50 μl of a reaction mixture containing 10 ng of the aforementioned Ogura-type radish cytoplasm DNA as the template, with 25 cycles of 95 °C for 30 seconds, 60 °C for 30 seconds, and 72 °C for 30 seconds. To each of the forward primers (−F), the T7 promoter sequence for synthesizing the substrate RNA in vitro was added.

Each of the obtained DNA fragments was purified by developing it on agarose gel, and then excising a gel section containing it. By allowing a reaction using the purified DNA fragment as the template at 37 °C for 60 minutes in 20 μl of a reaction mixture containing NTP mix (10 nmol GTP, CPT, ATP, and 0.5 nmol UTP), 4 μl [32^{P}]α-UTP (GE Healthcare, 3000 Ci/mmol), and T7 RNA polymerase (Takara Bio), a substrate RNA was synthesized.

The substrate RNA was subjected to phenol/chloroform extraction and ethanol precipitation, and then the total amount thereof was developed by electrophoresis on denatured 6% polyacrylamide gel containing 6 M urea, and the ³²P-labeled RNA was detected by exposing the gel to an X-ray film for 60 seconds.

Then, the section of the ³²P-labeled RNA was excised from the gel, and immersed in 200 μl of a gel elution solution (0.3 M sodium acetate, 2.5 mM EDTA, 0.01% SDS) at 4 °C for 12 hours to elute the RNA from the gel. The radioactivity of 1 μl of the RNA fraction was measured, and the total amount of the synthesized RNA was calculated. The RNA solution was subjected to ethanol precipitation, and then the RNA was dissolved in ultrapure water at 2500 cpm/μl (1 fmol/μl). By this preparation method, about 100 μl of RNA of 2500 cpm/μl was usually obtained.

### (5-5) Binding Experiment of Protein and RNA

Recombinant proteins of Enko B (Rf), Kosena B (rf), and Enko A (rf, ORF687-like protein existing in the Enko variety) were prepared, and the RNA binding activities thereof were verified.

The RNA binding activities of the prepared recombinant proteins (Enko B (SEQ ID NO: 576), Kosena B (SEQ ID NO: 577), and Enko A (SEQ ID NO: 578)) were analyzed by the gel shift assay. The aforementioned substrate RNA (BD120, 375 pM, 7.5 fmol/20 μL) and 0 to 2500 nM of each recombinant protein were mixed in 20 μl of a reaction mixture (10 mM Tris-HCl (pH 7.9), 30 mM KCl, 6 mM MgCl₂, 2 mM DTT, 8% glycerol, 0.0067% Triton X-100), and the reaction was allowed at 25 °C for 15 minutes. Then, 4 μl of a 80% glycerol solution was added to the reaction mixture, 10 μL of the mixture was developed on 10% non-denatured polyacrylamide gel containing 1×TBE (89 mM Tris-HCl, 89 mM boric acid, 2 mM EDTA), and after the electrophoresis, the gel was dried.

The radioactivity of RNA in the gel was measured with Bioimaging Analyzer BAS2000 (Fuji Photo Film).

### Example 6: RNA Binding Experiment Using Recombinant Proteins

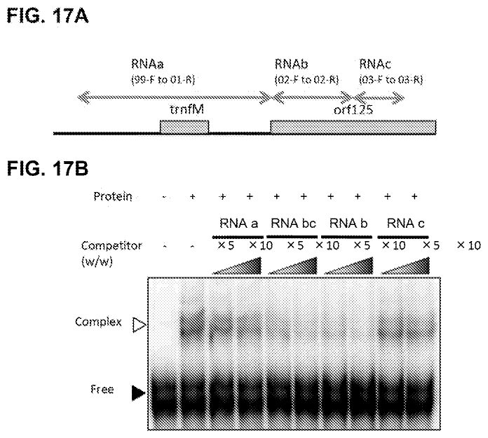

FIGS. 17A-17B show the analysis of binding of the Enko B protein and RNA containing the cytoplasmic male sterility (CMS) gene. FIG. 17A shows a schematic diagram around the mitochondrial orf125, and also schematically shows the regions of RNAa, RNAbc, RNAb, and RNAc used in the binding experiment. FIG. 17B shows binding of the Enko B protein and RNA. Enko B protein (1.4 nmol) and ³²P-labeled RNAbc (0.1 ng) were reacted in the presence of non-labeled RNAa, RNAbc, RNAb, and RNAc (×5 and ×10 w/w with respect to RNAbc, used as a competitive inhibition substance) in 20 μL of a reaction mixture to perform the gel shift competition experiment. Complex Δ mentioned on the left side of the diagram indicates the complex of the protein and RNA, and Free ▴ indicates RNA itself.

As shown in the drawings, the binding of the protein and RNA is visualized as a difference in the migration degree of the ³²P-labeled RNA. This is because the molecular weight of the complex of the ³²P-labeled RNA and the protein is larger than the molecular weight of the ³²P-labeled RNA alone, and therefore the migration degree thereof in the electrophoresis becomes smaller.

In this experiment, a recombinant protein of Enko B was prepared, and binding thereof with a mitochondrial RNA containing orf125 was verified by competition gel shift assay. RI-labeled RNAb and the protein were mixed, and then non-labeled RNA was added. That is, a more reduced signal intensity of the band at the position indicated as Complex means that RNA at that position added as a competitor and the protein binds, i.e., the position corresponds to an RNA region to which Enko B binds with higher affinity. As a result, it was revealed that Enko B strongly binds to the region of RNAb.

The candidate sequence of No. 208 shows the most significant P value in the binding sequence prediction shown in FIGS. 19A-19B, and correctly locates at the 3′ end of tRNA methionine. However, the analyses so far revealed that there is no difference in amount of tRNA and configuration of RNA containing orf125 (presence or absence of cleavage) between sterile and fertility-restored pedigrees, and the in vitro binding experiment (FIG. 17B) revealed that the RNAa sequence containing the sequence of No. 208 and Enko B do not bind. Therefore, it was judged that this region is not involved in fertility and sterility of Ogura-type cytoplasm.

Accordingly, further analysis was focused on the regions of Nos. 316, 352 and 373 contained in RNAb. RNAb consists of 125 b. Although it was attempted to narrow down the binding region to a 20 b order by using scanning mutation, it could not be limited to a single site (data are now shown). Therefore, it was considered that a plurality of binding sites for Enko B might exist in RNAb.

### Example 7: RNA Binding Activity of Rf-Like Proteins

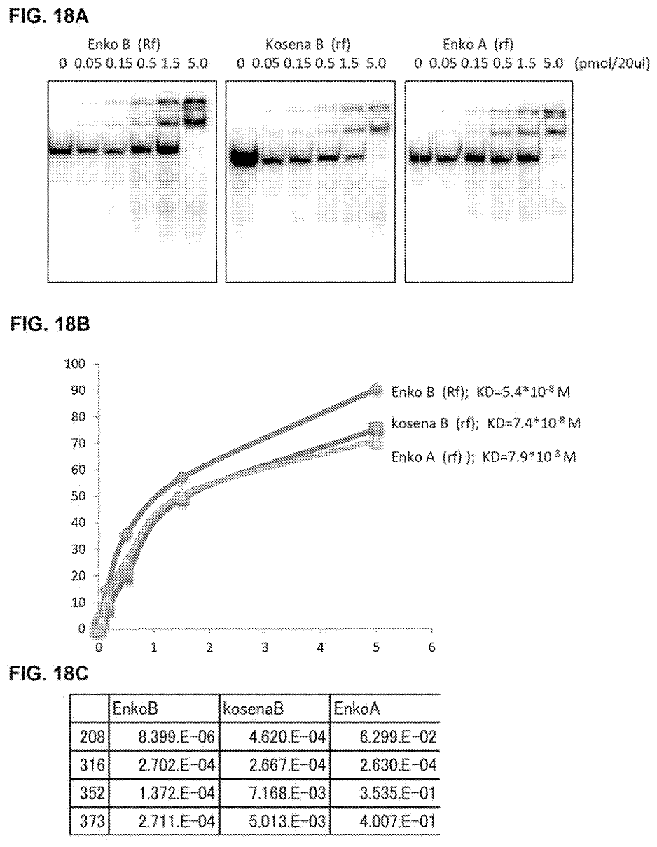

FIGS. 18A-18C binding of ORF687-like proteins and RNA. FIG. 18A shows the results of analysis of RNA binding characteristics of ORF687-like proteins performed by gel shift assay for binding of Enko B (Rf), Kosena B (rf), and Enko A (rf) with RNAb. FIG. 18B is shows the results of FIG. 18A in the form of graph, and dissociation constants (KD) of the proteins representing the RNA binding capacities thereof were calculated on the basis of this graph. FIG. 18C shows the results of calculation of the matching scores of Enko B (Rf), Kosena B (rf), and Enko A (rf), and potential binding sites thereof performed in the same manner as that used for obtaining the results shown in FIGS. 19A-19B.

As a result, in the non-competing state, all of the three kinds of proteins (Enko B, Kosena B, and Enko A) bound to RNAb with high affinity. As for Kosena B, the RNA binding activity was analyzed in the competing state, but definite difference of the activity was not observed compared with that observed for Enko B (FIGS. 18A and 18B).

Kosena B often shows an RNA binding activity slightly lower than that of Enko B (lower by about 2 times in terms of KD). However, 10 times or more of difference of the activity is detected in many cases for general RNA binding, and the above difference cannot be regarded as a significant difference.

The proteins do not show definite difference of matching scores for the corresponding regions also in the prediction based on the PPR codes (FIG. 18C). Therefore, it was decided to examine a possibility that the difference of Enko B and Kosena B might originate in difference of actions exerted after binding, not in simple difference in RNA binding affinity.

Further, prediction of binding sequences of a fertility restoration factor that acts on the Ogura-type cytoplasm are shown in FIGS. 19A-19B. FIG. 19A shows the results for prediction of binding of the Enko B protein using the PPR codes, and the structure of RNA containing the CMS gene orf125 is shown in the lower diagram of FIG. 19A. As for the regions from RNAa to RNAc shown in FIG. 19A, refer to FIG. 17. In FIG. 19A, the regions of Nos. 208, 230, 316, 352 and 373 are focused on, among the regions that showed a significantly high P value (FIG. 19A).

Further, sequence logos of the target RNA sequences predicted from the ORF687 protein sequence (sequences of the regions that showed a significant P value (Nos. 208, 316, 352, 373)), candidate binding RNA sequences, and sequence logos of the target RNA sequences predicted from the sequence of the ORF687-like protein of the radish variety having a recessive rf, Kosena (Kosena B) are shown in FIG. 18B. Further, the predicted binding base of Kosena B, which is a recessive rf, is also shown.

It was revealed that the bases specified by EnkoB and Kosena B are different (UA in the case of Rf, and GC in the case of rf), because of the amino acid polymorphism in the 2nd and 3rd PPR motifs. It could be predicted that this difference is directly linked with the functional difference between Rf and rf.

### Example 8: Prediction and Analysis of RNA Structure

On the basis of computerized prediction and in vitro RNA binding experiment, there was contemplated a possibility that Rf binds the region of RNAb, especially the regions of Nos. 316, 352 and 373. On the basis of the in vitro analysis, there was also contemplated a possibility that RNAb has a plurality of binding sites. Therefore, the secondary structure of the RNAb sequence was predicted, and attention was paid to the regions.

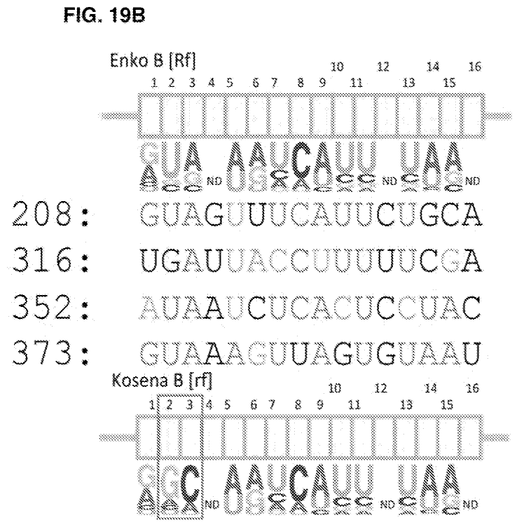

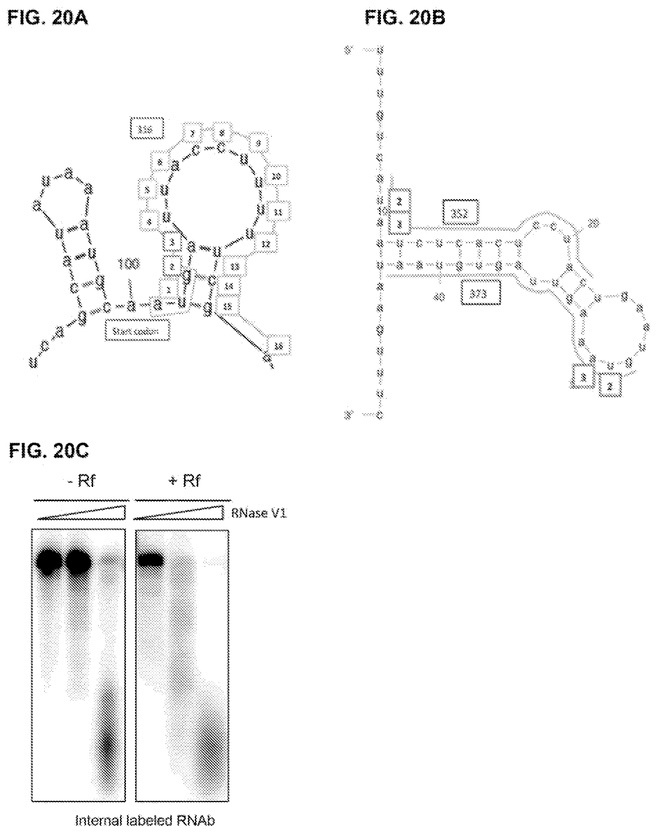

The results are shown in FIGS. 20A-20C. FIGS. 20A-20C show the secondary structure and structural change of the candidate binding RNA regions of ORF687-like protein. FIG. 20A shows the secondary structure of the region including the region of No. 306 and the predicted binding sites for the ORF687-like protein, and shows PPR motifs with boxes together with the corresponding bases. The 2nd and 3rd PPR motifs for which Enko B (Rf) and Kosena B (rf) show a remarkable difference are emphasized. FIG. 20B shows the secondary structure of the region including the regions of Nos. 352 and 373 and the predicted binding sites for the ORF687-like protein. FIG. 20C shows results indicating structural change of RNAb induced by Enko B, which were obtained by mixing RNAb and Enko B protein, and then adding a double-strand selective RNase (RNase V1).

As a result, it was revealed that the No. 316 region corresponds to the stem loop structure immediately downstream from the start codon of orf125 (FIG. 20A). Further, the 2nd and 3rd PPR motifs showing polymorphism between Enko B and Kosena B located in the double-strand at the root of the stem loop. In particular, the base corresponding the 3rd PPR motif is A in Enko B, whereas it is C in Kosena B (refer to FIG. 19B). On the basis of these results, there was contemplated a working hypothesis that Enko B binds to the region concerned to promote formation of the stem loop structure, and thereby inhibit translation of orf125.

A double-strand structure is also predicted for the Nos. 352 and 373 regions, and it was contemplated that the Rf protein binds on the both sides (FIG. 20B). However, in such a case, it is expected that the structure will be destroyed by the binding of Rf (formation of single strand is promoted). Further, differences in corresponding base and structure were not contemplated for the 2nd and 3rd PPR motifs, for which Rf and rf show difference, and any specific molecular mechanism could not be predicted.

Therefore, internally-labeled RNA was mixed with the proteins, and RNase V1 was added to the mixture to decompose only the labeled RNA. RNase V1 is an RNase that selectively cleaves only double-strand regions of RNA. As a result, it was demonstrated that the substrate RNA is more quickly decomposed in the presence of the protein, namely, formation of double-stranded RNA is promoted in the presence of Rf (Enko B) (FIG. 20C). That is, it was considered that the translation inhibition based on the formation of double-stranded RNA in orf125 mRNA by Rf is the major cause of the fertility restoration in Ogura-type cytoplasmic male sterility.

### Example 9: Determination of Function for Fertility Restoration Capacity of ORF687-Like Gene

ORF687-like genes have so far been isolated from various radish varieties, and the functionality thereof as Rf is estimated on the basis of mating experiments. However, the encoded amino acid sequences are very alike, and therefore it is impossible to determine the functionality as Rf from the conservation characteristics of the whole amino acid sequences.

In this example, sequences of the ORF687-like proteins were first analyzed. Specifically, the protein sequences shown in SEQ ID NOS: 576 to 578 and 585 to 591 were used as materials, and the sequences of them as the PPR proteins were analyzed. By using all the sequences as query sequences for CLUSTALW (http://www.genome.jp/tools/clustalw/), sequence alignment was obtained. By using the domain analysis software usable on the Web:
Pfam (http://pfam.sanger.ac.uk/),
InterProScan (http://www.ebi.ac.uk/Tools/InterProScan/), and
Prosite (http://www.expasy.org/prosite/),
   alignment of the ORF687-like proteins was created, and the PPR motif structures of the proteins were analyzed. The results are shown in FIGS. 21A-21B. All the ORF687-like proteins each consist of 16 PPR motifs (FIGS. 21A-21B).

From the obtained PPR motif models, amino acids 1, 2, and “ii” (−2) according to the amino acid numbers shown in Non-patent document 5 were extracted, and used for determination of the function for the fertility restoration ability of the ORF-like proteins.

Thus, functions of the 9 kinds of Rf-like genes were determined by using the PPR codes. The amino acids responsible for the base specifying capacity (1, 4, and ii) were extracted in the same manner as that used for Enko B mentioned above, converted into PPR codes, and used for determination of the functionality thereof by using the amino acid species as RNA binding windows (FIG. 22). Although Enko B and Kosena B show a homology of 99.4% for the whole sequences, two of RNA binding windows show amino acid polymorphism, and it was considered that they were deeply involved in dominance and recessiveness for the fertility restoration by the ORF687-like genes (Non-patent reference 4). Further, the gene Comet B locating on the same locus as that of Enko B in the variety Comet shows a homology of 98.0% with respect to Enko B, and the RNA binding windows of them are completely the same. The finding that Comet B is a dominant gene obtained by the past mating tests could be verified. Further, Enko A is an overlapping gene locating near Enko B, and it was suggested also from the viewpoint of RNA recognition that it is a recessive gene. These data suggest that, for the dominance and recessiveness for the fertility restoration of the ORF687-like genes, it is important that the amino acids responsible for the base specifying capacity (1, 4, ii) are the same in all the corresponding PPR motifs in the ORF687-like genes, in particular, they have the same amino acids 4 (A₄), or the same amino acids “ii”. Inter alia, it is considered that it is especially important that they have the same amino acids 4 (A₄). From this point of view, it was considered that the genes locating on the same locus as that of Enko B in various pedigrees of radish, of which information concerning fertility is unknown, rrORF690-1, rrORF690-2, icicle_pprCA, PC_PPR-A, and PC_PPR-BL, have RNA binding windows different from those of Enko B, which is a dominant gene, and these genes are also recessive rf.

The results described above suggest that the PPR codes used in the present invention can accelerate the determination of functions of industrially useful PPR proteins, which act as a fertility restoration factor. When a new pedigree is used for the first filial hybrid breeding method using the CMS-Rf system, whether candidate Rf gene sequences have fertility restoration ability can be determined from the sequences thereof by the above technique. The inventors of the present invention determined functions of the ORF687-like genes of 21 kinds of novel radish varieties, and successfully determined whether the fertility restoration ability of the ORF-like gene is dominant or recessive for 19 varieties (data are not shown). This technique can be applied not only to radish of the Ogura-type cytoplasm, but also to various cytoplasms and plant varieties containing a PPR protein as Rf.

### REFERENCES CITED IN EXAMPLES

Reference 1: Small, I. D., and Peeters, N. (2000), The PPR motif-a TPR-related motif prevalent in plant organellar proteins, Trends Biochem. Sci., 25, 46-47
Reference 2: Lurin, C., Andres, C., Aubourg, S., Bellaoui, M., Bitton, F., Bruyere, C., Caboche, M., Debast, C., Gualberto, J., Hoffmann, B., et al. (2004), Genome-wide analysis of Arabidopsis pentatricopeptide repeat proteins reveals their essential role in organelle biogenesis, Plant Cell, 16, 2089-2103
Reference 3: Okuda, K., Myouga, F., Motohashi, R., Shinozaki, K., and Shikanai, T. (2007), Conserved domain structure of pentatricopeptide repeat proteins involved in chloroplast RNA editing, Proc. Natl. Acad. Sci. USA, 104, 8178-8183
Reference 4: Koizuka N, Imai R, Fujimoto H, Hayakawa T, Kimura Y, et al. (2003), Genetic characterization of a pentatricopeptide repeat protein gene, orf687, that restores fertility in the cytoplasmic male-sterile Kosena radish, Plant J., 34:407-415
Reference 5: Nakamura T, Yagi Y, Kobayashi K. (2012), Mechanistic insight into pentatricopeptide repeat proteins as sequence-specific RNA-binding proteins for organellar RNAs in plants, Plant & Cell Physiology, 53:1171-1179.

What is claimed is:

1. A method for modifying or disrupting a nucleic acid having a target sequence, or imparting a novel function to a nucleic acid having a target sequence, in a living body or cell, the method comprising:
determining a nucleic acid sequence coding for an amino acid sequence of a protein,
cloning said nucleic acid sequence, and
preparing a vector carrying a polynucleotide consisting of the nucleic acid sequence,
wherein the protein contains one or more of PPR motifs, each consisting of a polypeptide of 30- to 38-amino acid length represented by the formula 1:
(HelixA)-X-(HelixB)-L (Formula 1)
wherein, in formula 1, Helix A is a moiety of 12-amino acid length capable of forming an α-helix structure, and is represented by the formula 2:
A₁-A₂-A₃-A₄-A₅-A₆-A₇-A₅-A₉-A₁₀-A₁₁-A₁₂ (Formula 2)
wherein, in the formula 2, A₁ to A₁₂ independently represent an amino acid;
wherein, in the formula 1, X is a moiety of 1- to 9-amino acid length and is optional;
wherein, in the formula 1, Helix B is a moiety of 11- to 13-amino acid length capable of forming an α-helix structure; and
wherein, in the formula 1, L is a moiety of 2- to 7-amino acid length represented by the formula 3;
Lᵥᵢᵢ-Lᵥᵢ-Lᵥ-Lᵢᵥ-Lᵢᵢᵢ-Lᵢᵢ-Lᵢ (Formula 3)
wherein, in the formula 3, Lᵢ to Lᵥᵢᵢ independently represent an amino acid, and Lᵢᵢᵢ to Lᵥᵢᵢ are optional, and
wherein a combination of three amino acids A₁, A₄ and Lᵢᵢ, or a combination of two amino acids A₄ and Lᵢᵢ corresponds to the target sequence;
preparing a cell containing the nucleic acid having the target sequence; and
introducing the vector into the cell, whereby the protein binds to the nucleic acid having the target sequence.

2. The method according to claim 1, wherein the combination of the three amino acids A₁, A₄ and Lᵢᵢ is determined according to any one of the following propositions:
(3-1) when the three amino acids A₁, A₄, and Lᵢᵢ are valine, asparagine, and aspartic acid, respectively, the PPR motif can selectively bind to U (uracil);
(3-2) when the three amino acids A₁, A₄, and Lᵢᵢ are valine, threonine, and asparagine, respectively, the PPR motif can selectively bind to A (adenine);
(3-3) when the three amino acids A₁, A₄, and Lᵢᵢ are valine, asparagine, and asparagine, respectively, the PPR motif can selectively bind to C (cytosine);
(3-4) when the three amino acids A₁, A₄, and Lᵢᵢ are glutamic acid, glycine, and aspartic acid, respectively, the PPR motif can selectively bind to G (guanine);
(3-5) when the three amino acids A₁, A₄, and Lᵢᵢ are isoleucine, asparagine, and asparagine, respectively, the PPR motif can selectively bind to C or U;
(3-6) when the three amino acids A₁, A₄, and Lᵢᵢ are valine, threonine, and aspartic acid, respectively, the PPR motif can selectively bind to G;
(3-7) when the three amino acids A₁, A₄, and Lᵢᵢ are lysine, threonine, and aspartic acid, respectively, the PPR motif can selectively bind to G;
(3-8) when the three amino acids A₁, A₄, and Lᵢᵢ are phenylalanine, serine, and asparagine, respectively, the PPR motif can selectively bind to A;
(3-9) when the three amino acids A₁, A₄, and Lᵢᵢ are valine, asparagine, and serine, respectively, the PPR motif can selectively bind to C;
(3-10) when the three amino acids A₁, A₄, and Lᵢᵢ are phenylalanine, threonine, and asparagine, respectively, the PPR motif can selectively bind to A;
(3-11) when the three amino acids A₁, A₄, and Lᵢᵢ are isoleucine, asparagine, and aspartic acid, respectively, the PPR motif can selectively bind to U or A;
(3-12) when the three amino acids A₁, A₄, and Lᵢᵢ are threonine, threonine, and asparagine, respectively, the PPR motif can selectively bind to A;
(3-13) when the three amino acids A₁, A₄, and Lᵢᵢ are isoleucine, methionine, and aspartic acid, respectively, the PPR motif can selectively bind to U or C;
(3-14) when the three amino acids A₁, A₄, and Lᵢᵢ are phenylalanine, proline, and aspartic acid, respectively, the PPR motif can selectively bind to U;
(3-15) when the three amino acids A₁, A₄, and Lᵢᵢ are tyrosine, proline, and aspartic acid, respectively, the PPR motif can selectively bind to U; and
(3-16) when the three amino acids A₁, A₄, and Lᵢᵢ are leucine, threonine, and aspartic acid, respectively, the PPR motif can selectively bind to G.

3. The method according to claim 1, wherein the combination of the two amino acids A₄ and Lᵢᵢ is determined according to any one of the following propositions:
(2-1) when A₄ and Lᵢᵢ are asparagine and aspartic acid, respectively, the motif can selectively bind to U;
(2-2) when A₄ and Lᵢᵢ are asparagine and asparagine, respectively, the motif can selectively bind to C;
(2-3) when A₄ and Lᵢᵢ are threonine and asparagine, respectively, the motif can selectively bind to A;
(2-4) when A₄ and Lᵢᵢ are threonine and aspartic acid, respectively, the motif can selectively bind to G;
(2-5) when A₄ and Lᵢᵢ are serine and asparagine, respectively, the motif can selectively bind to A;
(2-6) when A₄ and Lᵢᵢ are glycine and aspartic acid, respectively, the motif can selectively bind to G;
(2-7) when A₄ and Lᵢᵢ are asparagine and serine, respectively, the motif can selectively bind to C;
(2-8) when A₄ and Lᵢᵢ are proline and aspartic acid, respectively, the motif can selectively bind to U;
(2-9) when A₄ and Lᵢᵢ are glycine and asparagine, respectively, the motif can selectively bind to A;
(2-10) when A₄ and Lᵢᵢ are methionine and aspartic acid, respectively, the motif can selectively bind to U;
(2-11) when A₄ and Lᵢᵢ are leucine and aspartic acid, respectively, the motif can selectively bind to C; and
(2-12) when A₄ and Lᵢᵢ are valine and threonine, respectively, the motif can selectively bind to U.

4. A method for modifying or disrupting a nucleic acid having a target sequence, or imparting a novel function to a nucleic acid having a target sequence, in a living body or cell, the method comprising:
preparing a vector carrying a polynucleotide consisting of a nucleic acid sequence coding for an amino acid sequence of a protein which contains one or more of PPR motifs, each consisting of a polypeptide of 30- to 38-amino acid length represented by the formula 1:
(HelixA)-X-(HelixB)-L (Formula 1)
wherein, in formula 1, Helix A is a moiety of 12-amino acid length capable of forming an α-helix structure, and is represented by the formula 2:
A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid;
wherein, in the formula 1, X is a moiety of 1- to 9-amino acid length and is optional;
wherein, in the formula 1, Helix B is a moiety of 11- to 13-amino acid length capable of forming an α-helix structure; and
wherein, in the formula 1, L is a moiety of 2- to 7-amino acid length represented by the formula 3;
Lᵥᵢᵢ-Lᵥᵢᵢ-Lᵥ-Lᵢᵥ-Lᵢᵢᵢ-Lᵢᵢ-Lᵢ (Formula 3)
wherein, in the formula 3, Lᵢ to Lᵥᵢᵢ independently represent an amino acid, and Lᵢᵢᵢ to Lᵥᵢᵢ are optional, and
wherein a combination of three amino acids A₁, A₄ and Lᵢᵢ, or a combination of two amino acids A₄ and Lᵢᵢ corresponds to the target sequence;
preparing a cell containing the nucleic acid having the target sequence; and
introducing the protein into the cell, whereby the protein binds to the nucleic acid having the target sequence.

5. The method according to claim 4, wherein the combination of the three amino acids A₁, A₄ and Lᵢᵢ is determined according to any one of the following propositions:
(3-1) when the three amino acids A₁, A₄, and Lᵢᵢ are valine, asparagine, and aspartic acid, respectively, the PPR motif can selectively bind to U (uracil);
(3-2) when the three amino acids A₁, A₄, and Lᵢᵢ are valine, threonine, and asparagine, respectively, the PPR motif can selectively bind to A (adenine);
(3-3) when the three amino acids A₁, A₄, and Lᵢᵢ are valine, asparagine, and asparagine, respectively, the PPR motif can selectively bind to C (cytosine);
(3-4) when the three amino acids A₁, A₄, and Lᵢᵢ are glutamic acid, glycine, and aspartic acid, respectively, the PPR motif can selectively bind to G (guanine);
(3-5) when the three amino acids A₁, A₄, and Lᵢᵢ are isoleucine, asparagine, and asparagine, respectively, the PPR motif can selectively bind to C or U;
(3-6) when the three amino acids A₁, A₄, and Lᵢᵢ are valine, threonine, and aspartic acid, respectively, the PPR motif can selectively bind to G;
(3-7) when the three amino acids A₁, A₄, and Lᵢᵢ are lysine, threonine, and aspartic acid, respectively, the PPR motif can selectively bind to G;
(3-8) when the three amino acids A₁, A₄, and Lᵢᵢ are phenylalanine, serine, and asparagine, respectively, the PPR motif can selectively bind to A;
(3-9) when the three amino acids A₁, A₄, and Lᵢᵢ are valine, asparagine, and serine, respectively, the PPR motif can selectively bind to C;
(3-10) when the three amino acids A₁, A₄, and Lᵢᵢ are phenylalanine, threonine, and asparagine, respectively, the PPR motif can selectively bind to A;
(3-11) when the three amino acids A₁, A₄, and Lᵢᵢ are isoleucine, asparagine, and aspartic acid, respectively, the PPR motif can selectively bind to U or A;
(3-12) when the three amino acids A₁, A₄, and Lᵢᵢ are threonine, threonine, and asparagine, respectively, the PPR motif can selectively bind to A;
(3-13) when the three amino acids A₁, A₄, and Lᵢᵢ are isoleucine, methionine, and aspartic acid, respectively, the PPR motif can selectively bind to U or C;
(3-14) when the three amino acids A₁, A₄, and Lᵢᵢ are phenylalanine, proline, and aspartic acid, respectively, the PPR motif can selectively bind to U;
(3-15) when the three amino acids A₁, A₄, and Lᵢᵢ are tyrosine, proline, and aspartic acid, respectively, the PPR motif can selectively bind to U; and
(3-16) when the three amino acids A₁, A₄, and Lᵢᵢ are leucine, threonine, and aspartic acid, respectively, the PPR motif can selectively bind to G.

6. The method according to claim 4, wherein the combination of the two amino acids A₄ and Lᵢᵢ is determined according to any one of the following propositions:
(2-1) when A₄ and Lᵢᵢ are asparagine and aspartic acid, respectively, the motif can selectively bind to U;
(2-2) when A₄ and Lᵢᵢ are asparagine and asparagine, respectively, the motif can selectively bind to C;
(2-3) when A₄ and Lᵢᵢ are threonine and asparagine, respectively, the motif can selectively bind to A;
(2-4) when A₄ and Lᵢᵢ are threonine and aspartic acid, respectively, the motif can selectively bind to G;
(2-5) when A₄ and Lᵢᵢ are serine and asparagine, respectively, the motif can selectively bind to A;
(2-6) when A₄ and Lᵢᵢ are glycine and aspartic acid, respectively, the motif can selectively bind to G;
(2-7) when A₄ and Lᵢᵢ are asparagine and serine, respectively, the motif can selectively bind to C;
(2-8) when A₄ and Lᵢᵢ are proline and aspartic acid, respectively, the motif can selectively bind to U;
(2-9) when A₄ and Lᵢᵢ are glycine and asparagine, respectively, the motif can selectively bind to A;
(2-10) when A₄ and Lᵢᵢ are methionine and aspartic acid, respectively, the motif can selectively bind to U;
(2-11) when A₄ and Lᵢᵢ are leucine and aspartic acid, respectively, the motif can selectively bind to C; and
(2-12) when A₄ and Lᵢᵢ are valine and threonine, respectively, the motif can selectively bind to U.

7. A method for controlling a function of a nucleic acid having a target sequence, comprising:
determining a nucleic acid sequence coding for an amino acid sequence of a protein, cloning said nucleic acid sequence, and
preparing a vector carrying a polynucleotide consisting of the nucleic acid sequence,
wherein the protein contains one or more of PPR motifs, each consisting of a polypeptide of 30- to 38-amino acid length represented by the formula 1:
(HelixA)-X-(HelixB)-L (Formula 1)
wherein, in formula 1, Helix A is a moiety of 12-amino acid length capable of forming an α-helix structure, and is represented by the formula 2:
A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid;
wherein, in the formula 1, X is a moiety of 1- to 9-amino acid length and is optional;
wherein, in the formula 1, Helix B is a moiety of 11- to 13-amino acid length capable of forming an α-helix structure; and
wherein, in the formula 1, L is a moiety of 2- to 7-amino acid length represented by the formula 3;
Lᵥᵢᵢ-Lᵥᵢᵢ-Lᵥ-Lᵢᵥ-Lᵢᵢᵢ-Lᵢᵢ-Lᵢ (Formula 3)
wherein, in the formula 3, Lᵢ to Lᵥᵢᵢ independently represent an amino acid, and Lᵢᵢᵢ to Lᵥᵢ are optional, and
wherein a combination of three amino acids A₁, A₄ and Lᵢᵢ, or a combination of two amino acids A₄ and Lᵢᵢ corresponds to the target sequence;
preparing a cell containing the nucleic acid having the target sequence; and
introducing the vector into the cell, whereby the protein binds to the nucleic acid having the target sequence.

8. The method according to claim 1, wherein the combination of the three amino acids A₁, A₄ and Lᵢᵢ is determined according to any one of the following propositions:
(3-1) when the three amino acids A₁, A₄, and Lᵢᵢ are valine, asparagine, and aspartic acid, respectively, the PPR motif can selectively bind to U (uracil);
(3-2) when the three amino acids A₁, A₄, and Lᵢᵢ are valine, threonine, and asparagine, respectively, the PPR motif can selectively bind to A (adenine);
(3-3) when the three amino acids A₁, A₄, and Lᵢᵢ are valine, asparagine, and asparagine, respectively, the PPR motif can selectively bind to C (cytosine);
(3-4) when the three amino acids A₁, A₄, and Lᵢᵢ are glutamic acid, glycine, and aspartic acid, respectively, the PPR motif can selectively bind to G (guanine);
(3-5) when the three amino acids A₁, A₄, and Lᵢᵢ are isoleucine, asparagine, and asparagine, respectively, the PPR motif can selectively bind to C or U;
(3-6) when the three amino acids A₁, A₄, and Lᵢᵢ are valine, threonine, and aspartic acid, respectively, the PPR motif can selectively bind to G;
(3-7) when the three amino acids A₁, A₄, and Lᵢᵢ are lysine, threonine, and aspartic acid, respectively, the PPR motif can selectively bind to G;
(3-8) when the three amino acids A₁, A₄, and Lᵢᵢ are phenylalanine, serine, and asparagine, respectively, the PPR motif can selectively bind to A;
(3-9) when the three amino acids A₁, A₄, and Lᵢᵢ are valine, asparagine, and serine, respectively, the PPR motif can selectively bind to C;
(3-10) when the three amino acids A₁, A₄, and Lᵢᵢ are phenylalanine, threonine, and asparagine, respectively, the PPR motif can selectively bind to A;
(3-11) when the three amino acids A₁, A₄, and Lᵢᵢ are isoleucine, asparagine, and aspartic acid, respectively, the PPR motif can selectively bind to U or A;
(3-12) when the three amino acids A₁, A₄, and Lᵢᵢ are threonine, threonine, and asparagine, respectively, the PPR motif can selectively bind to A;
(3-13) when the three amino acids A₁, A₄, and Lᵢᵢ are isoleucine, methionine, and aspartic acid, respectively, the PPR motif can selectively bind to U or C;
(3-14) when the three amino acids A₁, A₄, and Lᵢᵢ are phenylalanine, proline, and aspartic acid, respectively, the PPR motif can selectively bind to U;
(3-15) when the three amino acids A₁, A₄, and Lᵢᵢ are tyrosine, proline, and aspartic acid, respectively, the PPR motif can selectively bind to U; and
(3-16) when the three amino acids A₁, A₄, and Lᵢᵢ are leucine, threonine, and aspartic acid, respectively, the PPR motif can selectively bind to G.

9. The method according to claim 1, wherein the combination of the two amino acids A₄ and Lᵢᵢ is determined according to any one of the following propositions:
(2-1) when A₄ and Lᵢᵢ are asparagine and aspartic acid, respectively, the motif can selectively bind to U;
(2-2) when A₄ and Lᵢᵢ are asparagine and asparagine, respectively, the motif can selectively bind to C;
(2-3) when A₄ and Lᵢᵢ are threonine and asparagine, respectively, the motif can selectively bind to A;
(2-4) when A₄ and Lᵢᵢ are threonine and aspartic acid, respectively, the motif can selectively bind to G;
(2-5) when A₄ and Lᵢᵢ are serine and asparagine, respectively, the motif can selectively bind to A;
(2-6) when A₄ and Lᵢᵢ are glycine and aspartic acid, respectively, the motif can selectively bind to G;
(2-7) when A₄ and Lᵢᵢ are asparagine and serine, respectively, the motif can selectively bind to C;
(2-8) when A₄ and Lᵢᵢ are proline and aspartic acid, respectively, the motif can selectively bind to U;
(2-9) when A₄ and Lᵢᵢ are glycine and asparagine, respectively, the motif can selectively bind to A;
(2-10) when A₄ and Lᵢᵢ are methionine and aspartic acid, respectively, the motif can selectively bind to U;
(2-11) when A₄ and Lᵢᵢ are leucine and aspartic acid, respectively, the motif can selectively bind to C; and
(2-12) when A₄ and Lᵢᵢ are valine and threonine, respectively, the motif can selectively bind to U.

10. A method for controlling a function of a nucleic acid having a target sequence, comprising:
preparing a vector carrying a polynucleotide consisting of a nucleic acid sequence coding for an amino acid sequence of a protein which contains one or more of PPR motifs, each consisting of a polypeptide of 30- to 38-amino acid length represented by the formula 1:
(HelixA)-X-(HelixB)-L (Formula 1)
wherein, in formula 1, Helix A is a moiety of 12-amino acid length capable of forming an α-helix structure, and is represented by the formula 2:
A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid;
wherein, in the formula 1, X is a moiety of 1- to 9-amino acid length and is optional;
wherein, in the formula 1, Helix B is a moiety of 11- to 13-amino acid length capable of forming an α-helix structure; and
wherein, in the formula 1, L is a moiety of 2- to 7-amino acid length represented by the formula 3;
Lᵥᵢᵢ-Lᵥᵢᵢ-Lᵥ-Lᵢᵥ-Lᵢᵢᵢ-Lᵢᵢ-Lᵢ (Formula 3)
wherein, in the formula 3, Lᵢ to Lᵥᵢ independently represent an amino acid, and Lᵢᵢᵢ to Lᵥᵢ are optional, and
wherein a combination of three amino acids A₁, A₄ and Lᵢᵢ, or a combination of two amino acids A₄ and Lᵢᵢ corresponds to the target sequence;
preparing a cell containing the nucleic acid having the target sequence; and
introducing the protein into the cell, whereby the protein binds to the nucleic acid having the target sequence.

11. The method according to claim 6, wherein the combination of the three amino acids A₁, A₄ and Lᵢᵢ is determined according to any one of the following propositions:
(3-1) when the three amino acids A₁, A₄, and Lᵢᵢ are valine, asparagine, and aspartic acid, respectively, the PPR motif can selectively bind to U (uracil);
(3-2) when the three amino acids A₁, A₄, and Lᵢᵢ are valine, threonine, and asparagine, respectively, the PPR motif can selectively bind to A (adenine);
(3-3) when the three amino acids A₁, A₄, and Lᵢᵢ are valine, asparagine, and asparagine, respectively, the PPR motif can selectively bind to C (cytosine);
(3-4) when the three amino acids A₁, A₄, and Lᵢᵢ are glutamic acid, glycine, and aspartic acid, respectively, the PPR motif can selectively bind to G (guanine);
(3-5) when the three amino acids A₁, A₄, and Lᵢᵢ are isoleucine, asparagine, and asparagine, respectively, the PPR motif can selectively bind to C or U;
(3-6) when the three amino acids A₁, A₄, and Lᵢᵢ are valine, threonine, and aspartic acid, respectively, the PPR motif can selectively bind to G;
(3-7) when the three amino acids A₁, A₄, and Lᵢᵢ are lysine, threonine, and aspartic acid, respectively, the PPR motif can selectively bind to G;
(3-8) when the three amino acids A₁, A₄, and Lᵢᵢ are phenylalanine, serine, and asparagine, respectively, the PPR motif can selectively bind to A;
(3-9) when the three amino acids A₁, A₄, and Lᵢᵢ are valine, asparagine, and serine, respectively, the PPR motif can selectively bind to C;
(3-10) when the three amino acids A₁, A₄, and Lᵢᵢ are phenylalanine, threonine, and asparagine, respectively, the PPR motif can selectively bind to A;
(3-11) when the three amino acids A₁, A₄, and Lᵢᵢ are isoleucine, asparagine, and aspartic acid, respectively, the PPR motif can selectively bind to U or A;
(3-12) when the three amino acids A₁, A₄, and Lᵢᵢ are threonine, threonine, and asparagine, respectively, the PPR motif can selectively bind to A;
(3-13) when the three amino acids A₁, A₄, and Lᵢᵢ are isoleucine, methionine, and aspartic acid, respectively, the PPR motif can selectively bind to U or C;
(3-14) when the three amino acids A₁, A₄, and Lᵢᵢ are phenylalanine, proline, and aspartic acid, respectively, the PPR motif can selectively bind to U;
(3-15) when the three amino acids A₁, A₄, and Lᵢᵢ are tyrosine, proline, and aspartic acid, respectively, the PPR motif can selectively bind to U; and
(3-16) when the three amino acids A₁, A₄, and Lᵢᵢ are leucine, threonine, and aspartic acid, respectively, the PPR motif can selectively bind to G.

12. The method according to claim 6, wherein the combination of the two amino acids A₄ and Lᵢᵢ is determined according to any one of the following propositions:
(2-1) when A₄ and Lᵢᵢ are asparagine and aspartic acid, respectively, the motif can selectively bind to U;
(2-2) when A₄ and Lᵢᵢ are asparagine and asparagine, respectively, the motif can selectively bind to C;
(2-3) when A₄ and Lᵢᵢ are threonine and asparagine, respectively, the motif can selectively bind to A;
(2-4) when A₄ and Lᵢᵢ are threonine and aspartic acid, respectively, the motif can selectively bind to G;
(2-5) when A₄ and Lᵢᵢ are serine and asparagine, respectively, the motif can selectively bind to A;
(2-6) when A₄ and Lᵢᵢ are glycine and aspartic acid, respectively, the motif can selectively bind to G;
(2-7) when A₄ and Lᵢᵢ are asparagine and serine, respectively, the motif can selectively bind to C;
(2-8) when A₄ and Lᵢᵢ are proline and aspartic acid, respectively, the motif can selectively bind to U;
(2-9) when A₄ and Lᵢᵢ are glycine and asparagine, respectively, the motif can selectively bind to A;
(2-10) when A₄ and Lᵢᵢ are methionine and aspartic acid, respectively, the motif can selectively bind to U;
(2-11) when A₄ and Lᵢᵢ are leucine and aspartic acid, respectively, the motif can selectively bind to C; and
(2-12) when A₄ and Lᵢᵢ are valine and threonine, respectively, the motif can selectively bind to U.

13. A method for controlling a function of nucleic acid, comprising:
preparing a vector carrying a polynucleotide consisting of the nucleic acid sequence coding for an amino acid sequence of a complex comprising a protein region consisting of a protein linked to a functional region, the protein containing one or more of PPR motifs, each consisting of a polypeptide of 30- to 38-amino acid length represented by the formula 1:
(HelixA)-X-(HelixB)-L (Formula 1)
wherein, in formula 1, Helix A is a moiety of 12-amino acid length capable of forming an α-helix structure, and is represented by the formula 2:
A₁-A₂-A₃-A₄-A₅-A₆-A₇-A₈-A₉-A₁₀-A₁₁-A₁₂ (Formula 2)
wherein, in the formula 2, A₁ to A₁₂ independently represent an amino acid;
wherein, in the formula 1, X is a moiety of 1- to 9-amino acid length and is optional;
wherein, in the formula 1, Helix B is a moiety of 11- to 13-amino acid length capable of forming an α-helix structure; and
wherein, in the formula 1, L is a moiety of 2- to 7-amino acid length represented by the formula 3;
Lᵥᵢᵢ-Lᵥᵢ-Lᵥ-Lᵢᵥ-Lᵢᵢᵢ-Lᵢᵢ-Lᵢ (Formula 3)
wherein, in the formula 3, Lᵢ to Lᵥᵢ independently represent an amino acid, and Lᵢᵢᵢ to Lᵥᵢᵢ are optional, and
wherein a combination of three amino acids A₁, A₄ and Lᵢᵢ, or a combination of two amino acids A₄ and Lᵢᵢ corresponds to the target sequence;
preparing a cell containing a nucleic acid having a target sequence; and
introducing the complex into the cell, whereby the protein region of the complex binds to the nucleic acid having the target sequence and the functional region modifies the function of the nucleic acid.

14. The method according to claim 11, wherein the combination of the three amino acids A₁, A₄ and Lᵢᵢ is determined according to any one of the following propositions:
(3-1) when the three amino acids A₁, A₄, and Lᵢᵢ are valine, asparagine, and aspartic acid, respectively, the PPR motif can selectively bind to U (uracil);
(3-2) when the three amino acids A₁, A₄, and Lᵢᵢ are valine, threonine, and asparagine, respectively, the PPR motif can selectively bind to A (adenine);
(3-3) when the three amino acids A₁, A₄, and Lᵢᵢ are valine, asparagine, and asparagine, respectively, the PPR motif can selectively bind to C (cytosine);
(3-4) when the three amino acids A₁, A₄, and Lᵢᵢ are glutamic acid, glycine, and aspartic acid, respectively, the PPR motif can selectively bind to G (guanine);
(3-5) when the three amino acids A₁, A₄, and Lᵢᵢ are isoleucine, asparagine, and asparagine, respectively, the PPR motif can selectively bind to C or U;
(3-6) when the three amino acids A₁, A₄, and Lᵢᵢ are valine, threonine, and aspartic acid, respectively, the PPR motif can selectively bind to G;
(3-7) when the three amino acids A₁, A₄, and Lᵢᵢ are lysine, threonine, and aspartic acid, respectively, the PPR motif can selectively bind to G;
(3-8) when the three amino acids A₁, A₄, and Lᵢᵢ are phenylalanine, serine, and asparagine, respectively, the PPR motif can selectively bind to A;
(3-9) when the three amino acids A₁, A₄, and Lᵢᵢ are valine, asparagine, and serine, respectively, the PPR motif can selectively bind to C;
(3-10) when the three amino acids A₁, A₄, and Lᵢᵢ are phenylalanine, threonine, and asparagine, respectively, the PPR motif can selectively bind to A;
(3-11) when the three amino acids A₁, A₄, and Lᵢᵢ are isoleucine, asparagine, and aspartic acid, respectively, the PPR motif can selectively bind to U or A;
(3-12) when the three amino acids A₁, A₄, and Lᵢᵢ are threonine, threonine, and asparagine, respectively, the PPR motif can selectively bind to A;
(3-13) when the three amino acids A₁, A₄, and Lᵢᵢ are isoleucine, methionine, and aspartic acid, respectively, the PPR motif can selectively bind to U or C;
(3-14) when the three amino acids A₁, A₄, and Lᵢᵢ are phenylalanine, proline, and aspartic acid, respectively, the PPR motif can selectively bind to U;
(3-15) when the three amino acids A₁, A₄, and Lᵢᵢ are tyrosine, proline, and aspartic acid, respectively, the PPR motif can selectively bind to U; and
(3-16) when the three amino acids A₁, A₄, and Lᵢᵢ are leucine, threonine, and aspartic acid, respectively, the PPR motif can selectively bind to G.

15. The method according to claim 11, wherein the combination of the two amino acids A₄ and Lᵢᵢ is determined according to any one of the following propositions:
(2-1) when A₄ and Lᵢᵢ are asparagine and aspartic acid, respectively, the motif can selectively bind to U;
(2-2) when A₄ and Lᵢᵢ are asparagine and asparagine, respectively, the motif can selectively bind to C;
(2-3) when A₄ and Lᵢᵢ are threonine and asparagine, respectively, the motif can selectively bind to A;
(2-4) when A₄ and Lᵢᵢ are threonine and aspartic acid, respectively, the motif can selectively bind to G;
(2-5) when A₄ and Lᵢᵢ are serine and asparagine, respectively, the motif can selectively bind to A;
(2-6) when A₄ and Lᵢᵢ are glycine and aspartic acid, respectively, the motif can selectively bind to G;
(2-7) when A₄ and Lᵢᵢ are asparagine and serine, respectively, the motif can selectively bind to C;
(2-8) when A₄ and Lᵢᵢ are proline and aspartic acid, respectively, the motif can selectively bind to U;
(2-9) when A₄ and Lᵢᵢ are glycine and asparagine, respectively, the motif can selectively bind to A;
(2-10) when A₄ and Lᵢᵢ are methionine and aspartic acid, respectively, the motif can selectively bind to U;
(2-11) when A₄ and Lᵢᵢ are leucine and aspartic acid, respectively, the motif can selectively bind to C; and
(2-12) when A₄ and Lᵢᵢ are valine and threonine, respectively, the motif can selectively bind to U.

16. A method for modifying a cellular genetic material, comprising:
preparing a cell containing a nucleic acid having a target sequence; and
introducing the vector defined in claim 13 into the cell,
whereby the protein region of the complex binds to the nucleic acid having the target sequence and the functional region modifies the target sequence.

17. The method according to claim 16, wherein the combination of the three amino acids A₁, A₄ and Lᵢᵢ is determined according to any one of the following propositions:
(3-1) when the three amino acids A₁, A₄, and Lᵢᵢ are valine, asparagine, and aspartic acid, respectively, the PPR motif can selectively bind to U (uracil);
(3-2) when the three amino acids A₁, A₄, and Lᵢᵢ are valine, threonine, and asparagine, respectively, the PPR motif can selectively bind to A (adenine);
(3-3) when the three amino acids A₁, A₄, and Lᵢᵢ are valine, asparagine, and asparagine, respectively, the PPR motif can selectively bind to C (cytosine);
(3-4) when the three amino acids A₁, A₄, and Lᵢᵢ are glutamic acid, glycine, and aspartic acid, respectively, the PPR motif can selectively bind to G (guanine);
(3-5) when the three amino acids A₁, A₄, and Lᵢᵢ are isoleucine, asparagine, and asparagine, respectively, the PPR motif can selectively bind to C or U;
(3-6) when the three amino acids A₁, A₄, and Lᵢᵢ are valine, threonine, and aspartic acid, respectively, the PPR motif can selectively bind to G;
(3-7) when the three amino acids A₁, A₄, and Lᵢᵢ are lysine, threonine, and aspartic acid, respectively, the PPR motif can selectively bind to G;
(3-8) when the three amino acids A₁, A₄, and Lᵢᵢ are phenylalanine, serine, and asparagine, respectively, the PPR motif can selectively bind to A;
(3-9) when the three amino acids A₁, A₄, and Lᵢᵢ are valine, asparagine, and serine, respectively, the PPR motif can selectively bind to C;
(3-10) when the three amino acids A₁, A₄, and Lᵢᵢ are phenylalanine, threonine, and asparagine, respectively, the PPR motif can selectively bind to A;
(3-11) when the three amino acids A₁, A₄, and Lᵢᵢ are isoleucine, asparagine, and aspartic acid, respectively, the PPR motif can selectively bind to U or A;
(3-12) when the three amino acids A₁, A₄, and Lᵢᵢ are threonine, threonine, and asparagine, respectively, the PPR motif can selectively bind to A;
(3-13) when the three amino acids A₁, A₄, and Lᵢᵢ are isoleucine, methionine, and aspartic acid, respectively, the PPR motif can selectively bind to U or C;
(3-14) when the three amino acids A₁, A₄, and Lᵢᵢ are phenylalanine, proline, and aspartic acid, respectively, the PPR motif can selectively bind to U;
(3-15) when the three amino acids A₁, A₄, and Lᵢᵢ are tyrosine, proline, and aspartic acid, respectively, the PPR motif can selectively bind to U; and
(3-16) when the three amino acids A₁, A₄, and Lᵢᵢ are leucine, threonine, and aspartic acid, respectively, the PPR motif can selectively bind to G.

18. The method according to claim 15, wherein the combination of the two amino acids A₄ and Lᵢᵢ is determined according to any one of the following propositions:
(2-1) when A₄ and Lᵢᵢ are asparagine and aspartic acid, respectively, the motif can selectively bind to U;
(2-2) when A₄ and Lᵢᵢ are asparagine and asparagine, respectively, the motif can selectively bind to C;
(2-3) when A₄ and Lᵢᵢ are threonine and asparagine, respectively, the motif can selectively bind to A;
(2-4) when A₄ and Lᵢᵢ are threonine and aspartic acid, respectively, the motif can selectively bind to G;
(2-5) when A₄ and Lᵢᵢ are serine and asparagine, respectively, the motif can selectively bind to A;
(2-6) when A₄ and Lᵢᵢ are glycine and aspartic acid, respectively, the motif can selectively bind to G;
(2-7) when A₄ and Lᵢᵢ are asparagine and serine, respectively, the motif can selectively bind to C;
(2-8) when A₄ and Lᵢᵢ are proline and aspartic acid, respectively, the motif can selectively bind to U;
(2-9) when A₄ and Lᵢᵢ are glycine and asparagine, respectively, the motif can selectively bind to A;
(2-10) when A₄ and Lᵢᵢ are methionine and aspartic acid, respectively, the motif can selectively bind to U;
(2-11) when A₄ and Lᵢᵢ are leucine and aspartic acid, respectively, the motif can selectively bind to C; and
(2-12) when A₄ and Lᵢᵢ are valine and threonine, respectively, the motif can selectively bind to U.


SEQUENCE LISTING (591 sequences)

SEQ ID NO: 1 - protein, 555 aa, Arabidopsis thaliana
MLRHAIETNVQIFTKFLVISASAVGIGYARKLFDQRPQRDDSFLSNSMIKAYLETRQYPDSFALYRDLRKETCFAPDNFTFTTLTKSCSLSMCVYQGLQLHSQIWRFGFCADMYVSTGVVDMYAKFGKMGCARNAFDEMPHRSEVSWTALISGYIRCGELDLASKLFDQMPHVKDVVIYNAMMDGFVKSGDMTSARRLFDEMTHKTVITWTTMIHGYCNIKDIDAARKLFDAMPERNLVSWNTMIGGYCQNKQPQEGIRLFQEMQATTSLDPDDVTILSVLPAISDTGALSLGEWCHCFVQRKKLDKKVKVCTAILDMYSKCGEIEKAKRIFDEMPEKQVASWNAMIHGYALNGNARAALDLFVTMMIEEKPDEITMLAVITACNHGGLVEEGRKWFHVMREMGLNAKIEHYGCMVDLLGRAGSLKEAEDLITNMPFEPNGIILSSFLSACGQYKDIERAERILKKAVELEPQNDGNYVLLRNLYAADKRWDDFGMVKNVMRKNQAKKEVGCSLIEINYIVSEFISGDTTHPHRRSIHLVLGDLLMHMNEEKYNW

SEQ ID NO: 2 - protein, 500 aa, Arabidopsis thaliana
MGLLPVVGITSPALITHKNHANPKIQRHNQSTSETTVSWTSRINLLTRNGRLAEAAKEFSDMTLAGVEPNHITFIALLSGCGDFTSGSEALGDLLHGYACKLGLDRNHVMVGTAIIGMYSKRGRFKKARLVFDYMEDKNSVTWNTMIDGYMRSGQVDNAAKMFDKMPERDLISWTAMINGFVKKGYQEEALLWFREMQISGVKPDYVAIIAALNACTNLGALSFGLWVHRYVLSQDFKNNVRVSNSLIDLYCRCGCVEFARQVFYNMEKRTVVSWNSVIVGFAANGNAHESLVYFRKMQEKGFKPDAVTFTGALTACSHVGLVEEGLRYFQIMKCDYRISPRIEHYGCLVDLYSRAGRLEDALKLVQSMPMKPNEVVIGSLLAACSNHGNNIVLAERLMKHLTDLNVKSHSNYVILSNMYAADGKWEGASKMRRKMKGLGLKKQPGFSSIEIDDCMHVFMAGDNAHVETTYIREVLELISSDLRLQGCVVETLAGDLLNA

SEQ ID NO: 3 - protein, 830 aa, Arabidopsis thaliana
MASLPFNTIPNKVPFSVSSKPSSKHHDEQAHSPSSTSYFHRVSSLCKNGEIKEALSLVTEMDFRNLRIGPEIYGEILQGCVYERDLSTGKQIHARILKNGDFYARNEYIETKLVIFYAKCDALEIAEVLFSKLRVRNVFSWAAIIGVKCRIGLCEGALMGFVEMLENEIFPDNFVVPNVCKACGALKWSRFGRGVHGYVVKSGLEDCVFVASSLADMYGKCGVLDDASKVFDEIPDRNAVAWNALMVGYVQNGKNEEAIRLFSDMRKQGVEPTRVTVSTCLSASANMGGVEEGKQSHAIAIVNGMELDNILGTSLLNFYCKVGLIEYAEMVFDRMFEKDVVTWNLIISGYVQQGLVEDAIYMCQLMRLEKLKYDCVTLATLMSAAARTENLKLGKEVQCYCIRHSFESDIVLASTVMDMYAKCGSIVDAKKVFDSTVEKDLILWNTLLAAYAESGLSGEALRLFYGMQLEGVPPNVITWNLIILSLLRNGQVDEAKDMFLQMQSSGIIPNLISWTTMMNGMVQNGCSEEAILFLRKMQESGLRPNAFSITVALSACAHLASLHIGRTIHGYIIRNLQHSSLVSIETSLVDMYAKCGDINKAEKVFGSKLYSELPLSNAMISAYALYGNLKEAIALYRSLEGVGLKPDNITITNVLSACNHAGDINQAIEIFTDIVSKRSMKPCLEHYGLMVDLLASAGETEKALRLIEEMPFKPDARMIQSLVASCNKQRKTELVDYLSRKLLESEPENSGNYVTISNAYAVEGSWDEVVKMREMMKAKGLKKKPGCSWIQITGEEGVHVFVANDKTHTRINEIQMMLALLLYDMGTGSK

SEQ ID NO: 4 - protein, 809 aa, Arabidopsis thaliana
MSSQLVQFSTVPQIPNPPSRHRHFLSERNYIPANVYEHPAALLLERCSSLKELRQILPLVFKNGLYQEHFFQTKLVSLFCRYGSVDEAARVFEPIDSKLNVLYHTMLKGFAKVSDLDKALQFFVRMRYDDVEPVVYNFTYLLKVCGDEAELRVGKEIHGLLVKSGFSLDLFAMTGLENMYAKCRQVNEARKVFDRMPERDLVSWNTIVAGYSQNGMARMALEMVKSMCEENLKPSFITIVSVLPAVSALRLISVGKEIHGYAMRSGFDSLVNISTALVDMYAKCGSLETARQLFDGMLERNVVSWNSMIDAYVQNENPKEAMLIFQKMLDEGVKPTDVSVMGALHACADLGDLERGRFIHKLSVELGLDRNVSVVNSLISMYCKCKEVDTAASMFGKLQSRTLVSWNAMILGFAQNGRPIDALNYFSQMRSRTVKPDTFTYVSVITAIAELSITHHAKWIHGVVMRSCLDKNVFVTTALVDMYAKCGAIMIARLIFDMMSERHVTTWNAMIDGYGTHGFGKAALELFEEMQKGTIKPNGVTFLSVISACSHSGLVEAGLKCFYMMKENYSIELSMDHYGAMVDLLGRAGRLNEAWDFIMQMPVKPAVNVYGAMLGACQIHKNVNFAEKAAERLFELNPDDGGYHVLLANIYRAASMWEKVGQVRVSMLRQGLRKTPGCSMVEIKNEVHSFFSGSTAHPDSKKIYAFLEKLICHIKEAGYVPDTNLVLGVENDVKEQLLSTHSEKLAISFGLLNTTAGTTIHVRKNLRVCADCHNATKYISLVTGREIVVRDMQRFHHFKNGACSCGDYW

SEQ ID NO: 5 - protein, 638 aa, Arabidopsis thaliana
MVVRSIIVSPPTTITYYHPMSIGLLVHPLSPHIPPASSPSASTAGNHHQRIFSLAETCSDMSQLKQLHAFTLRTTYPEEPATLFLYGKILQLSSSFSDVNYAFRVFDSIENHSSFMWNTLIRACAHDVSRKEEAFMLYRKMLERGESSPDKHTFPFVLKACAYIFGFSEGKQVHCQIVKHGFGGDVYVNNGLIHLYGSCGCLDLARKVFDEMPERSLVSWNSMIDALVRFGEYDSALQLFREMQRSFEPDGYTMQSVLSACAGLGSLSLGTWAHAFLLRKCDVDVAMDVLVKNSLIEMYCKCGSLRMAEQVFQGMQKRDLASWNAMILGFATHGRAEEAMNFFDRMVDKRENVRPNSVTFVGLLIACNHRGFVNKGRQYFDMMVRDYCIEPALEHYGCIVDLIARAGYITEAIDMVMSMPMKPDAVIWRSLLDACCKKGASVELSEEIARNIIGTKEDNESSNGNCSGAYVLLSRVYASASRWNDVGIVRKLMSEHGIRKEPGCSSIEINGISHEFFAGDTSHPQTKQIYQQLKVIDDRLRSIGYLPDRSQAPLVDATNDGSKEYSLRLHSERLAIAFGLINLPPQTPIRIFKNLRVCNDCHEVTKLISKVFNTEIIVRDRVRFHHFKDGSCSCLDYW

SEQ ID NO: 6 - protein, 613 aa, Arabidopsis thaliana
MLVFKSTMECSISSTIHVLGSCKTSDDVNQIHGRLIKTGIIKNSNLTTRIVLAFASSRRPYLADFARCVFHEYHVCSFSFGEVEDPFLWNAVIKSHSHGKDPRQALLLLCLMLENGVSVDKFSLSLVLKACSRLGFVKGGMQIHGFLKKTGLWSDLFLQNCLIGLYLKCGCLGLSRQMFDRMPKRDSVSYNSMIDGYVKCGLIVSARELFDLMPMEMKNLISWNSMISGYAQTSDGVDIASKLFADMPEKDLISWNSMIDGYVKHGRIEDAKGLFDVMPRRDVVTWATMIDGYAKLGFVHHAKTLFDQMPHRDVVAYNSMMAGYVQNKYHMEALEIFSDMEKESHLLPDDTTLVIVLPAIAQLGRLSKAIDMHLYIVEKQFYLGGKLGVALIDMYSKCGSIQHAMLVFEGIENKSIDHWNAMIGGLAIHGLGESAFDMLLQIERLSLKPDDITFVGVLNACSHSGLVKEGLLCFELMRRKHKIEPRLQHYGCMVDILSRSGSIELAKNLIEEMPVEPNDVIWRTFLTACSHHKEFETGELVAKHLILQAGYNPSSYVLLSNMYASFGMWKDVRRVRTMMKERKIEKIPGCSWIELDGRVHEFFVDSIEVSSTL

SEQ ID NO: 7 - protein, 684 aa, Arabidopsis thaliana
MSLLSADALGLLLKNAISASSMRLGRVVHARIVKTLDSPPPPFLANYLINMYSKLDHPESARLVLRLTPARNVVSWTSLISGLAQNGHFSTALVEFFEMRREGVVPNDFTFPCAFKAVASLRLPVTGKQIHALAVKCGRILDVFVGCSAFDMYCKTRLRDDARKLFDEIPERNLETWNAFISNSVTDGRPREAIEAFIEFRRIDGHPNSITFCAFLNACSDWLHLNLGMQLHGLVLRSGFDTDVSVCNGLIDFYGKCKQIRSSEIIFTEMGTKNAVSWCSLVAAYVQNHEDEKASVLYLRSRKDIVETSDFMISSVLSACAGMAGLELGRSIHAHAVKACVERTIFVGSALVDMYGKCGCIEDSEQAFDEMPEKNLVTRNSLIGGYAHQGQVDMALALFEEMAPRGCGPTPNYMTFVSLLSACSRAGAVENGMKIFDSMRSTYGIEPGAEHYSCIVDMLGRAGMVERAYEFIKKMPIQPTISVWGALQNACRMHGKPQLGLLAAENLFKLDPKDSGNHVLLSNTFAAAGRWAEANTVREELKGVGIKKGAGYSWITVKNQVHAFQAKDRSHILNKEIQTTLAKLRNEMEAAGYKPDLKLSLYDLEEEEKAAEVSHHSEKLALAFGLLSLPLSVPIRITKNLRICGDCHSFFKFVSGSVKREIIVRDNNRFHRFKDGICSCKDYW

SEQ ID NO: 8 - protein, 646 aa, Arabidopsis thaliana
MNPTQTLFSPGGNSPASSPASHPSSLFPQINNCRTIRDLSQIHAVFIKSGQMRDTLAAAEILRFCATSDLHHRDLDYAHKIFNQMPQRNCFSWNTIIRGFSESDEDKALIAITLFYEMMSDEFVEPNRFTFPSVLKACAKTGKIQEGKQIHGLALKYGFGGDEFVMSNLVRMYVMCGFMKDARVLFYKNIIEKDMVVMTDRRKRDGEIVLWNVMIDGYMRLGDCKAARMLFDKMRQRSVVSWNTMISGYSLNGFFKDAVEVFREMKKGDIRPNYVTLVSVLPAISRLGSLELGEWLHLYAEDSGIRIDDVLGSALIDMYSKCGIIEKAIHVFERLPRENVITWSAMINGFAIHGQAGDAIDCFCKMRQAGVRPSDVAYINLLTACSHGGLVEEGRRYFSQMVSVDGLEPRIEHYGCMVDLLGRSGLLDEAEEFILNMPIKPDDVIWKALLGACRMQGNVEMGKRVANILMDMVPHDSGAYVALSNMYASQGNWSEVSEMRLRMKEKDIRKDPGCSLIDIDGVLHEFVVEDDSHPKAKEINSMLVEISDKLRLAGYRPITTQVLLNLEEEDKENVLHYHSEKIATAFGLISTSPGKPIRIVKNLRICEDCHSSIKLISKVYKRKITVRDRKRFHHFQDGSCSCMDYW

SEQ ID NO: 9 - protein, 588 aa, Arabidopsis thaliana
MALNSSAFFVPCHNYNQICDLLLSSARTRSTIKGLQLHGYVVKSGLSLIPLVANNLINFYSKSQLPFDSRRAFEDSPQKSSTTWSSIISCFAQNELPWMSLEFLKKMMAGNLRPDDHVLPSATKSCAILSRCDIGRSVHCLSMKTGYDADVFVGSSLVDMYAKCGEIVYARKMFDEMPQRNVVTWSGMMYGYAQMGENEEALWLFKEALFENLAVNDYSFSSVISVCANSTLLELGRQIHGLSIKSSFDSSSFVGSSLVSLYSKCGVPEGAYQVFNEVPVKNLGIWNAMLKAYAQHSHTQKVIELFKRMKLSGMKPNFITFLNVLNACSHAGLVDEGRYYFDQMKESRIEPTDKHYASLVDMLGRAGRLQEALEVITNMPIDPTESVWGALLTSCTVHKNTELAAFAADKVFELGPVSSGMHISLSNAYAADGRFEDAAKARKLLRDRGEKKETGLSWVEERNKVHTFAAGERRHEKSKEIYEKLAELGEEMEKAGYIADTSYVLREVDGDEKNQTIRYHSERLAIAFGLITFPADRPIRVMKNLRVCGDCHNAIKFMSVCTRRVIIVRDNNRFHRFEDGKCSCNDYW

SEQ ID NO: 10 - protein, 659 aa, Arabidopsis thaliana
MKVRSKKALFCSVSRLLHTERHTERQNLTTLFNRYVDKTDVFSWNSVIADLARSGDSAEALLAFSSMRKLSLYPTRSSFPCAIKACSSLFDIFSGKQTHQQAFVFGYQSDIFVSSALIVMYSTCGKLEDARKVFDEIPKRNIVSWTSMIRGYDLNGNALDAVSLFKDLLVDENDDDDAMFLDSMGLVSVISACSRVPAKGLTESIHSFVIKRGFDRGVSVGNTLLDAYAKGGEGGVAVARKIFDQIVDKDRVSYNSIMSVYAQSGMSNEAFEVFRRLVKNKVVTFNAITLSTVLLAVSHSGALRIGKCIHDQVIRMGLEDDVIVGTSIIDMYCKCGRVETARKAFDRMKNKNVRSWTAMIAGYGMHGHAAKALELFPAMIDSGVRPNYITFVSVLAACSHAGLHVEGWRWFNAMKGRFGVEPGLEHYGCMVDLLGRAGFLQKAYDLIQRMKMKPDSIIWSSLLAACRIHKNVELAEISVARLFELDSSNCGYYMLLSHIYADAGRWKDVERVRMIMKNRGLVKPPGFSLLELNGEVHVFLIGDEEHPQREKIYEFLAELNRKLLEAGYVSNTSSVCHDVDEEEKEMTLRVHSEKLAIAFGIMNTVPGSTVNVVKNLRVCSDCHNVIKLISKIVDREFVVRDAKRFHHFKDGGCSCGDYW

SEQ ID NO: 11 - protein, 685 aa, Arabidopsis thaliana
MIKLIRFFRSRRCWVISLQARCFSAPSRTHFDFSGESSDTERALVSALGSCASSNDVTCGRQIHCRVLKSGLDSNGYICNSVLNMYAKCRLLADAESVFRDHAKLDSASFNIMVDGYVRSRRLWDALKLFDVMPERSCVSYTTLIKGYAQNNQWSEAMELFREMRNLGIMLNEVTLATVISACSHLGGIWDCRMLQSLAIKLKLEGRVFVSTNLLHMYCLCLCLKDARKLFDEMPERNLVTWNVMLNGYSKAGLIEQAEELFDQITEKDIVSWGTMIDGCLRKNQLDEALVYYTEMLRCGMKPSEVMMVDLLSASARSVGSSKGLQLHGTIVKRGFDCYDFLQATIIHFYAVSNDIKLALQQFEASVKDHIASRNALIAGFVKNGMVEQAREVFDQTHDKDIFSWNAMISGYAQSLSPQLALHLFREMISSSQVKPDAITMVSVFSAISSLGSLEEGKRAHDYLNFSTIPPNDNLTAAIIDMYAKCGSIETALNIFHQTKNISSSTISPWNAIICGSATHGHAKLALDLYSDLQSLPIKPNSITFVGVLSACCHAGLVELGKTYFESMKSDHGIEPDIKHYGCMVDLLGKAGRLEEAKEMIKKMPVKADVMIWGMLLSASRTHGNVEIAELAATELAAIDPSHGGCKVMLSNVYADAGRWEDVALVREEMRTRDVEWSRAFSGVV

SEQ ID NO: 12 - protein, 565 aa, Arabidopsis thaliana
MMKKHYKPILSQLENCRSLVELNQLHGLMIKSSVIRNVIPLSRLIDFCTTCPETMNLSYARSVFESIDCPSVYIWNSMIRGYSNSPNPDKALIFYQEMLRKGYSPDYFTFPYVLKACSGLRDIQFGSCVHGFVVKTGFEVNMYVSTCLLHMYMCCGEVNYGLRVFEDIPQWNVVAWGSLISGFVNNNRFSDAIEAFREMQSNGVKANETIMVDLLVACGRCKDIVTGKWFHGFLQGLGFDPYFQSKVGFNVILATSLIDMYAKCGDLRTARYLFDGMPERTLVSWNSIITGYSQNGDAEEALCMFLDMLDLGIAPDKVTFLSVIRASMIQGCSQLGQSIHAYVSKTGFVKDAAIVCALVNMYAKTGDAESAKKAFEDLEKKDTIAWTVVIIGLASHGHGNEALSIFQRMQEKGNATPDGITYLGVLYACSHIGLVEEGQRYFAEMRDLHGLEPTVEHYGCMVDILSRAGRFEEAERLVKTMPVKPNVNIWGALLNGCDIHENLELTDRIRSMVAEPEELGSGIYVLLSNIYAKAGRWADVKLIRESMKSKRVDKVLGHSSVETMF

SEQ ID NO: 13 - protein, 472 aa, Arabidopsis thaliana
MSSVFPGPRFLSLLQQNSKTLIQAKQIHAQLVINGCHDNSLFGKLIGHYCSKPSTESSSKLAHLLVFPRFGHPDKFLFNTLLKCSKPEDSIRIFANYASKSSLLYLNERTFVFVLGACARSASSSALRVGRIVHGMVKKLGFLYESELIGTTLLHFYAKNGDLRYARKVFDEMPERTSVTWNAMIGGYCSHKDKGNHNARKAMVLFRRFSCCGSGVRPTDTTMVCVLSAISQTGLLEIGSLVHGYIEKLGFTPEVDVFIGTALVDMYSKCGCLNNAFSVFELMKVKNVFTWTSMATGLALNGRGNETPNLLNRMAESGIKPNEITFTSLLSAYRHIGLVEEGIELFKSMKTRFGVTPVIEHYGCIVDLLGKAGRIQEAYQFILAMPIKPDAILLRSLCNACSIYGETVMGEEIGKALLEIEREDEKLSGSECEDYVALSNVLAHKGKWVEVEKLRKEMKERRIKTRPGYSFV

SEQ ID NO: 14 - protein, 534 aa, Arabidopsis thaliana
MAFHGIREVENYFIPFLQRVKSRNEWKKINASIIIHGLSQSSFMVTKMVDFCDKIEDMDYATRLFNQVSNPNVFLYNSIIRAYTHNSLYCDVIRIYKQLLRKSFELPDRFTFPFMFKSCASLGSCYLGKQVHGHLCKFGPRFHVVTENALIDMYMKFDDLVDAHKVFDEMYERDVISWNSLLSGYARLGQMKKAKGLFHLMLDKTIVSWTAMISGYTGIGCYVEAMDFFREMQLAGIEPDEISLISVLPSCAQLGSLELGKWIHLYAERRGFLKQTGVCNALIEMYSKCGVISQAIQLFGQMEGKDVISWSTMISGYAYHGNAHGAIETFNEMQRAKVKPNGITFLGLLSACSHVGMWQEGLRYFDMMRQDYQIEPKIEHYGCLIDVLARAGKLERAVEITKTMPMKPDSKIWGSLLSSCRTPGNLDVALVAMDHLVELEPEDMGNYVLLANIYADLGKWEDVSRLRKMIRNENMKKTPGGSLIEVNNIVQEFVSGDNSKPFWTEISIVLQLFTSHQDQDVITNNNALAFIGIV

SEQ ID NO: 15 - protein, 694 aa, Arabidopsis thaliana
MSEASCLASPLLYTNSGIHSDSFYASLIDSATHKAQLKQIHARLLVLGLQFSGFLITKLIHASSSFGDITFARQVFDDLPRPQIFPWNAIIRGYSRNNHFQDALLMYSNMQLARVSPDSFTFPHLLKACSGLSHLQMGRFVHAQVFRLGFDADVFVQNGLIALYAKCRRLGSARTVFEGLPLPERTIVSWTAIVSAYAQNGEPMEALEIFSQMRKMDVKPDWVALVSVLNAFTCLQDLKQGRSIHASVVKMGLEIEPDLLISLNTMYAKCGQVATAKILFDKMKSPNLILWNAMISGYAKNGYAREAIDMFHEMINKDVRPDTISITSAISACAQVGSLEQARSMYEYVGRSDYRDDVFISSALIDMFAKCGSVEGARLVFDRTLDRDVVVWSAMIVGYGLHGRAREAISLYRAMERGGVHPNDVTFLGLLMACNHSGMVREGWWFFNRMADHKINPQQQHYACVIDLLGRAGHLDQAYEVIKCMPVQPGVTVWGALLSACKKHRHVELGEYAAQQLFSIDPSNTGHYVQLSNLYAAARLWDRVAEVRVRMKEKGLNKDVGCSWVEVRGRLEAFRVGDKSHPRYEEIERQVEWIESRLKEGGFVANKDASLHDLNDEEAEETLCSHSERIAIAYGLISTPQGTPLRITKNLRACVNCHAATKLISKLVDREIVVRDTNRFHHFKDGVCSCGDYW

SEQ ID NO: 16 - protein, 656 aa, Arabidopsis thaliana
MIRSRSVLLIFRKVLYQSSCLKCLLCANSFSTSVSSSLGFRATNKELNQMIRSGYIAEARDIFEKLEARNTVTWNTMISGYVKRREMNQARKLFDVMPKRDVVTWNTMISGYVSCGGIRFLEEARKLFDEMPSRDSFSWNTMISGYAKNRRIGEALLLFEKMPERNAVSWSAMITGFCQNGEVDSAVVLFRKMPVKDSSPLCALVAGLIKNERLSEAAWVLGQYGSLVSGREDLVYAYNTLIVGYGQRGQVEAARCLFDQIPDLCGDDHGGEFRERFCKNVVSWNSMIKAYLKVGDVVSARLLFDQMKDRDTISWNTMIDGYVHVSRMEDAFALFSEMPNRDAHSWNMMVSGYASVGNVELARHYFEKTPEKHTVSWNSIIAAYEKNKDYKEAVDLFIRMNIEGEKPDPHTLTSLLSASTGLVNLRLGMQMHQIVVKTVIPDVPVHNALITMYSRCGEIMESRRIFDEMKLKREVITWNAMIGGYAFHGNASEALNLFGSMKSNGIYPSHITFVSVLNACAHAGLVDEAKAQFVSMMSVYKIEPQMEHYSSLVNVTSGQGQFEEAMYIITSMPFEPDKTVWGALLDACRIYNNVGLAHVAAEAMSRLEPESSTPYVLLYNMYADMGLWDEASQVRMNMESKRIKKERGSSWVDSST

SEQ ID NO: 17 - protein, 544 aa, Arabidopsis thaliana
MISSLAAITGGPSTFRRDPDSNTLRLSRRKTLISVLRSCKNIAHVPSIHAKIIRTFHDQDAFVVFELIRVCSTLDSVDYAYDVFSYVSNPNVYLYTAMIDGFVSSGRSADGVSLYHRMIHNSVLPDNYVITSVLKACDLKVCREIHAQVLKLGFGSSRSVGLKMMEIYGKSGELVNAKKMFDEMPDRDHVAATVMINCYSECGFIKEALELFQDVKIKDTVCWTAMIDGLVRNKEMNKALELFREMQMENVSANEFTAVCVLSACSDLGALELGRWVHSFVENQRMELSNFVGNALINMYSRCGDINEARRVFRVMRDKDVISYNTMISGLAMHGASVEAINEFRDMVNRGFRPNQVTLVALLNACSHGGLLDIGLEVFNSMKRVFNVEPQIEHYGCIVDLLGRVGRLEEAYRFIENIPIEPDHIMLGTLLSACKIHGNMELGEKIAKRLFESENPDSGTYVLLSNLYASSGKWKESTEIRESMRDSGIEKEPGCSTIEVDNQIHEFLVGDIAHPHKEAIYQRLQELNRILRFKENQIDIIMGF

SEQ ID NO: 18 - protein, 738 aa, Arabidopsis thaliana
MAIFSTAQPLSLPRHPNFSNPNQPTTNNERSRHISLIERCVSLRQLKQTHGHMIRTGTFSDPYSASKLFAMAALSSFASLEYARKVFDEIPKPNSFAWNTLIRAYASGPDPVLSIWAFLDMVSESQCYPNKYTFPFLIKAAAEVSSLSLGQSLHGMAVKSAVGSDVFVANSLIHCYFSCGDLDSACKVFTTIKEKDVVSWNSMINGFVQKGSPDKALELFKKMESEDVKASHVTMVGVLSACAKIRNLEFGRQVCSYIEENRVNVNLTLANAMLDMYTKCGSIEDAKRLFDAMEEKDNVTWTTMLDGYAISEDYEAAREVLNSMPQKDIVAWNALISAYEQNGKPNEALIVFHELQLQKNMKLNQITLVSTLSACAQVGALELGRWIHSYIKKHGIRMNFHVTSALIHMYSKCGDLEKSREVFNSVEKRDVFVWSAMIGGLAMHGCGNEAVDMFYKMQEANVKPNGVTFTNVFCACSHTGLVDEAESLFHQMESNYGIVPEEKHYACIVDVLGRSGYLEKAVKFIEAMPIPPSTSVWGALLGACKIHANLNLAEMACTRLLELEPRNDGAHVLLSNIYAKLGKWENVSELRKHMRVTGLKKEPGCSSIEIDGMIHEFLSGDNAHPMSEKVYGKLHEVMEKLKSNGYEPEISQVLQIIEEEEMKEQSLNLHSEKLAICYGLISTEAPKVIRVIKNLRVCGDCHSVAKLISQLYDREIIVRDRYRFHHFRNGQCSCNDFW

SEQ ID NO: 19 - protein, 741 aa, Arabidopsis thaliana
MMLSCSPLTVPSSSYPFHFLPSSSDPPYDSIRNHPSLSLLHNCKTLQSLRIIHAQMIKIGLHNTNYALSKLIEFCILSPHFEGLPYAISVFKTIQEPNLLIWNTMFRGHALSSDPVSALKLYVCMISLGLLPNSYTFPFVLKSCAKSKAFKEGQQIHGHVLKLGCDLDLYVHTSLISMYVQNGRLEDAHKVFDKSPHRDVVSYTALIKGYASRGYIENAQKLFDEIPVKDVVSWNAMISGYAETGNYKEALELFKDMMKTNVRPDESTMVTVVSACAQSGSIELGRQVHLWIDDHGFGSNLKIVNALIDLYSKCGELETACGLFERLPYKDVISWNTLIGGYTHMNLYKEALLLFQEMLRSGETPNDVTMLSILPACAHLGAIDIGRWIHVYIDKRLKGVTNASSLRTSLIDMYAKCGDIEAAHQVFNSILHKSLSSWNAMIFGFAMHGRADASFDLFSRMRKIGIQPDDITFVGLLSACSHSGMLDLGRHIFRTMTQDYKMTPKLEHYGCMIDLLGHSGLFKEAEEMINMMEMEPDGVIWCSLLKACKMHGNVELGESFAENLIKIEPENPGSYVLLSNIYASAGRWNEVAKTRALLNDKGMKKVPGCSSIEIDSVVHEFIIGDKFHPRNREIYGMLEEMEVLLEKAGFVPDTSEVLQEMEEEWKEGALRHHSEKLAIAFGLISTKPGTKLTIVKNLRVCRNCHEATKLISKIYKREIIARDRTRFHHFRDGVCSCNDYW

SEQ ID NO: 20 - protein, 890 aa, Arabidopsis thaliana
MSCPLAFTFSLPSIFPFPSQLLPFSRHKHPYLLRATPTSATEDVASAVSGAPSIFISQSRSPEWWIDLLRSKVRSNLLREAVLTYVDMIVLGIKPDNYAFPALLKAVADLQDMELGKQIHAHVYKFGYGVDSVTVANTLVNLYRKCGDFGAVYKVFDRISERNQVSWNSLISSLCSFEKWEMALEAFRCMLDENVEPSSFTLVSVVTACSNLPMPEGLMMGKQVHAYGLRKGELNSFIINTLVAMYGKLGKLASSKVLLGSFGGRDLVTWNTVLSSLCQNEQLLEALEYLREMVLEGVEPDEFTISSVLPACSHLEMLRTGKELHAYALKNGSLDENSFVGSALVDMYCNCKQVLSGRRVFDGMFDRKIGLWNAMIAGYSQNEHDKEALLLFIGMEESAGLLANSTTMAGVVPACVRSGAFSRKEAIHGFVVKRGLDRDRFVQNTLMDMYSRLGKIDIAMRIFGKMEDRDLVTWNTMITGYVFSEHHEDALLLLHKMQNLERKVSKGASRVSLKPNSITLMTILPSCAALSALAKGKEIHAYAIKNNLATDVAVGSALVDMYAKCGCLQMSRKVFDQIPQKNVITWNVIIMAYGMHGNGQEAIDLLRMMMVQGVKPNEVTFISVFAACSHSGMVDEGLRIFYVMKPDYGVEPSSDHYACVVDLLGRAGRIKEAYQLMNMMPRDFNKAGAWSSLLGASRIHNNLEIGEIAAQNLIQLEPNVASHYVLLANIYSSAGLWDKATEVRRNMKEQGVRKEPGCSWIEHGDEVHKFVAGDSSHPQSEKLSGYLETLWERMRKEGYVPDTSCVLHNVEEDEKEILLCGHSEKLAIAFGILNTSPGTIIRVAKNLRVCNDCHLATKFISKIVDREIILRDVRRFHRFKNGTCSCGDYW

SEQ ID NO: 21 - protein, 603 aa, Arabidopsis thaliana
MAISSASLISSFSHAETFTKHSKIDTVNTQNPILLISKCNSLRELMQIQAYAIKSHIEDVSFVAKLINFCTESPTESSMSYARHLFEAMSEPDIVIFNSMARGYSRFTNPLEVFSLFVEILEDGILPDNYTFPSLLKACAVAKALEEGRQLHCLSMKLGLDDNVYVCPTLINMYTECEDVDSARCVFDRIVEPCVVCYNAMITGYARRNRPNEALSLFREMQGKYLKPNEITLLSVLSSCALLGSLDLGKWIHKYAKKHSFCKYVKVNTALIDMFAKCGSLDDAVSIFEKMRYKDTQAWSAMIVAYANHGKAEKSMLMFERMRSENVQPDEITFLGLLNACSHTGRVEEGRKYFSQMVSKFGIVPSIKHYGSMVDLLSRAGNLEDAYEFIDKLPISPTPMLWRILLAACSSHNNLDLAEKVSERIFELDDSHGGDYVILSNLYARNKKWEYVDSLRKVMKDRKAVKVPGCSSIEVNNVVHEFFSGDGVKSATTKLHRALDEMVKELKLSGYVPDTSMVVHANMNDQEKEITLRYHSEKLAITFGLLNTPPGTTIRVVKNLRVCRDCHNAAKLISLIFGRKVVLRDVQRFHHFEDGKCSCGDFW

SEQ ID NO: 22 - protein, 960 aa, Arabidopsis thaliana
MEYAVTNMRLLSNMMYSASAISFPRVRLHCSIPTEPSCRRNPFRQSNQPVQVPSPKLACFDGVLTEAFQRLDVSENNSPVEAFAYVLELCGKRRAVSQGRQLHSRIFKTFPSFELDFLAGKLVFMYGKCGSLDDAEKVFDEMPDRTAFAWNTMIGAYVSNGEPASALALYWNMRVEGVPLGLSSFPALLKACAKLRDIRSGSELHSLLVKLGYHSTGFIVNALVSMYAKNDDLSAARRLFDGFQEKGDAVLWNSILSSYSTSGKSLETLELFREMHMTGPAPNSYTIVSALTACDGFSYAKLGKEIHASVLKSSTHSSELYVCNALIAMYTRCGKMPQAERILRQMNNADVVTWNSLIKGYVQNLMYKEALEFFSDMIAAGHKSDEVSMTSIIAASGRLSNLLAGMELHAYVIKHGWDSNLQVGNTLIDMYSKCNLTCYMGRAFLRMHDKDLISWTTVIAGYAQNDCHVEALELFRDVAKKRMEIDEMILGSILRASSVLKSMLIVKEIHCHILRKGLLDTVIQNELVDVYGKCRNMGYATRVFESIKGKDVVSWTSMISSSALNGNESEAVELFRRMVETGLSADSVALLCILSAAASLSALNKGREIHCYLLRKGFCLEGSIAVAVVDMYACCGDLQSAKAVFDRIERKGLLQYTSMINAYGMHGCGKAAVELFDKMRHENVSPDHISFLALLYACSHAGLLDEGRGFLKIMEHEYELEPWPEHYVCLVDMLGRANCVVEAFEFVKMMKTEPTAEVWCALLAACRSHSEKEIGEIAAQRLLELEPKNPGNLVLVSNVFAEQGRWNDVEKVRAKMKASGMEKHPGCSWIEMDGKVHKFTARDKSHPESKEIYEKLSEVTRKLEREVGYVADTKFVLHNVDEGEKVQMLHGHSERIAIAYGLLRTPDRACLRITKNLRVCRDCHTFCKLVSKLFRRDIVMRDANRFHHFESGLCSCGDSW

SEQ ID NO: 23 - protein, 895 aa, Arabidopsis thaliana
MNCLANESLNSLKISPFSTSRLLSSVTNFRNQLSFSSKDSSSSSAPFNPFRFFNDQSNSRLCNLRTTKILQAHLLRRYLLPFDVFLTKSLLSWYSNSGSMADAAKLFDTIPQPDVVSCNIMISGYKQHRLFEESLRFFSKMHFLGFEANEISYGSVISACSALQAPLFSELVCCHTIKMGYFFYEVVESALIDVFSKNLRFEDAYKVFRDSLSANVYCWNTIIAGALRNQNYGAVFDLFHEMCVGFQKPDSYTYSSVLAACASLEKLRFGKVVQARVIKCGAEDVFVCTAIVDLYAKCGHMAEAMEVFSRIPNPSVVSWTVMLSGYTKSNDAFSALEIFKEMRHSGVEINNCTVTSVISACGRPSMVCEASQVHAWVFKSGFYLDSSVAAALISMYSKSGDIDLSEQVFEDLDDIQRQNIVNVMITSFSQSKKPGKAIRLFTRMLQEGLRTDEFSVCSLLSVLDCLNLGKQVHGYTLKSGLVLDLTVGSSLFTLYSKCGSLEESYKLFQGIPFKDNACWASMISGFNEYGYLREAIGLFSEMLDDGTSPDESTLAAVLTVCSSHPSLPRGKEIHGYTLRAGIDKGMDLGSALVNMYSKCGSLKLARQVYDRLPELDPVSCSSLISGYSQHGLIQDGFLLFRDMVMSGFTMDSFAISSILKAAALSDESSLGAQVHAYITKIGLCTEPSVGSSLLTMYSKFGSIDDCCKAFSQINGPDLIAWTALIASYAQHGKANEALQVYNLMKEKGFKPDKVTFVGVLSACSHGGLVEESYFHLNSMVKDYGIEPENRHYVCMVDALGRSGRLREAESFINNMHIKPDALVWGTLLAACKIHGEVELGKVAAKKAIELEPSDAGAYISLSNILAEVGEWDEVEETRKLMKGTGVQKEPGWSSV

SEQ ID NO: 24 - protein, 681 aa, Arabidopsis thaliana
MNISKAKLLLLPPPLTPKLNRSLYSHSQRRTRSLPHHRDKPINWNSTHSFVLHNPLLSLLEKCKLLLHLKQIQAQMIINGLILDPFASSRLIAFCALSESRYLDYSVKILKGIENPNIFSWNVTIRGFSESENPKESFLLYKQMLRHGCCESRPDHFTYPVLFKVCADLRLSSLGHMILGHVLKLRLELVSHVHNASIHMFASCGDMENARKVFDESPVRDLVSWNCLINGYKKIGEAEKAIYVYKLMESEGVKPDDVTMIGLVSSCSMLGDLNRGKEFYEYVKENGLRMTIPLVNALMDMFSKCGDIHEARRIFDNLEKRTIVSWTTMISGYARCGLLDVSRKLFDDMEEKDVVLWNAMIGGSVQAKRGQDALALFQEMQTSNTKPDEITMIHCLSACSQLGALDVGIWIHRYIEKYSLSLNVALGTSLVDMYAKCGNISEALSVFHGIQTRNSLTYTAIIGGLALHGDASTAISYFNEMIDAGIAPDEITFIGLLSACCHGGMIQTGRDYFSQMKSRFNLNPQLKHYSIMVDLLGRAGLLEEADRLMESMPMEADAAVWGALLFGCRMHGNVELGEKAAKKLLELDPSDSGIYVLLDGMYGEANMWEDAKRARRMMNERGVEKIPGCSSIEVNGIVCEFIVRDKSRPESEKIYDRLHCLGRHMRSSLSVLFSEYEITNN

SEQ ID NO: 25 - protein, 842 aa, Arabidopsis thaliana
MAMLGNVLHLSPMVLATTTTTKPSLLNQSKCTKATPSSLKNCKTIDELKMFHRSLTKQGLDNDVSTITKLVARSCELGTRESLSFAKEVFENSESYGTCFMYNSLIRGYASSGLCNEAILLFLRMMNSGISPDKYTFPFGLSACAKSRAKGNGIQIHGLIVKMGYAKDLFVQNSLVHFYAECGELDSARKVFDEMSERNVVSWTSMICGYARRDFAKDAVDLFFRMVRDEEVTPNSVTMVCVISACAKLEDLETGEKVYAFIRNSGIEVNDLMVSALVDMYMKCNAIDVAKRLFDEYGASNLDLCNAMASNYVRQGLTREALGVFNLMMDSGVRPDRISMLSAISSCSQLRNILWGKSCHGYVLRNGFESWDNICNALIDMYMKCHRQDTAFRIFDRMSNKTVVTWNSIVAGYVENGEVDAAWETFETMPEKNIVSWNTIISGLVQGSLFEEAIEVFCSMQSQEGVNADGVTMMSIASACGHLGALDLAKWIYYYIEKNGIQLDVRLGTTLVDMFSRCGDPESAMSIFNSLTNRDVSAWTAAIGAMAMAGNAERAIELFDDMIEQGLKPDGVAFVGALTACSHGGLVQQGKEIFYSMLKLHGVSPEDVHYGCMVDLLGRAGLLEEAVQLIEDMPMEPNDVIWNSLLAACRVQGNVEMAAYAAEKIQVLAPERTGSYVLLSNVYASAGRWNDMAKVRLSMKEKGLRKPPGTSSIQIRGKTHEFTSGDESHPEMPNIEAMLDEVSQRASHLGHVPDLSNVLMDVDEKEKIFMLSRHSEKLAMAYGLISSNKGTTIRIVKNLRVCSDCHSFAKFASKVYNREIILRDNNRFHYIRQGKCSCGDFW

SEQ ID NO: 26 - protein, 859 aa, Physcomitrella patens
MLHQGAGRARGGLPSLLLGRRFDLWASFPWSFSSTSDDPSSRQSEENSQTIDELWEYFAGPSQWWDNRIHKRNPRSPDLKHKVTGKALWIDGCFTPEWVKFQPVAQVGLQSYATCTTKCTEGGAQGKRQPKGNDGKLATACESARVLGRIAQGGINMHVQTANTLSEAIVVLMNRLQRGLITDSFMYVEVLKRCLKQKDLMAAKQVHDCIIKSRMEQNAHVMNNLLHVYIECGRLQEARCVFDALVKKSGASWNAMIAGYVEHKHAEDAMRLFREMCHEGVQPNAGTYMIILKACASLSALKWGKEVHACIRHGGLESDVRVGTALLRMYGKCGSINEARRIFDNLMNHDIISWTVMIGAYAQSGNGKEAYRLMLQMEQEGFKPNAITYVSILNACASEGALKWVKRVHRHALDAGLELDVRVGTALVQMYAKSGSIDDARVVFDRMKVRDVVSWNVMIGAFAEHGRGHEAYDLFLQMQTEGCKPDAIMFLSILNACASAGALEWVKKIHRHALDSGLEVDVRVGTALVHMYSKSGSIDDARVVFDRMKVRNVVSWNAMISGLAQHGLGQDALEVFRRMTAHGVKPDRVTFVAVLSACSHAGLVDEGRSQYLAMTQVYGIEPDVSHCNCMVDLLGRAGRLMEAKLFIDNMAVDPDEATWGALLGSCRTYGNVELGELVAKERLKLDPKNAATYVLLSNIYAEAGKWDMVSWVRTMMRERGIRKEPGRSWIEVDNKIHDFLVADSSHPECKEINESKDKVIEKIKAEGYIPDTRLVLKNKNMKDKELDICSHSEKLAIVYGLMHTPPGNPIRVFKNLRVCTDCHGATKLISKVEGREIIVRDANRFHHFKDGVCSCGDYW

SEQ ID NO: 27 - protein, 833 aa, Physcomitrella patens
MRVSTRIAANIAVVRRRWFGSLQLPVPSARFRSTFTRRVGANDVLQRLGEGGNHIDSRTYVKLFQRCTELRDAALGKQVRDHIIQGGRQLNIYELNTLIKLYSICGNVTEARQIFDSVENKTVVTWNALIAGYAQVGHVKEAFALFRQMVDEGLEPSIITFLSVLDACSSPAGLNWGKEVHAQVVTAGFVSDFRIGTALVSMYVKGGSMDDARQVFDGLHIRDVSTFNVMVGGYAKSGDWEKAFELFYRMQQVGLKPNKISFLSILDGCWTPEALAWGKAVHAQCMNAGLVDDIRVATSLIRMYTTCGSIEGARRVFDNMKVRDVVSWTVMIEGYAENGNIEDAFGLFATMQEEGIQPDRITYMHIMNACAISANLNHAREIHSQVDIAGFGTDLLVSTALVHMYAKCGAIKDARQVFDAMPRRDVVSWSAMIGAYVENGYGTEAFETFHLMKRSNIEPDGVTYINLLNACGHLGALDVGMEIYTQAIKADLVSHVPLGNALIIMNAKHGSVERARYIFDTMVRRDVITWNAMIGGYSLHGNAREALYLFDRMLKERFRPNSVTFVGVLSACSRAGFVDEGRRFFTYLLEGRGIVPTVKLYGCMVDLLGRAGELDEAELLIKSMPVKPTSSIWSSLLVACRIHGNLDVAERAAERCLMIDPYDGAVYVQLSHMYAAAGMWENVAKVRKVMESRGIRKEQGCTWIEVAGKVHTFVVEDRSHPLVGEIYAELARLMNAIKREGYIPITQNVLHDVGEQQKEEAISYHSEKLAIAYGVLSLPSGTPIRIYKNLRVCSDCHSASKFISKVTGREIIARDASRFHHFKDGVCSCGDYW

SEQ ID NO: 28 - protein, 1204 aa, Physcomitrella patens
MYRCLWRSVIQTHKGSLGRVAGVREFSARPWPVEQNRSSIGAAGGESSNLVPVKVMRNERHCGPDREDVSNTHQPRPTETDRATYVALLQNCTRKRLLMRNERHCGPDREDVSNTHQPRPTETERATYVALLQNCTRKRLLPEAKRIHAQMVEAWVGPDIFLSNLLINMYVKCRSVLDAHQVFKEMPRRDVISWNSLISCYAQQGFKKKAFQLFEEMQNAGFIPNKITYISILTACYSPAELENGKKIHSQIIKAGYQRDPRVQNSLLSMYGKCGDLPRARQVFAGISPRDVVSYNTMLGLYAQKAYVKECLGLFGQMSSEGISPDKVTYINLLDAFTTPSMLDEGKRIHKLTVEEGLNSDIRVGTALVTMCVRCGDVDSAKQAFKGTADRDVVVYNALIAALAQHGHNVEAFEQYYRMRSDGVALNRTTYLSILNACSTSKALEAGKLIHSHISEDGHSSDVQIGNALISMYARCGDLPKARELFYTMPKRDLISWNAIIAGYARREDRGEAMRLYKQMQSEGVKPGRVTFLHLLSACANSSAYADGKMIHEDILRSGIKSNGHLANALMNMYRRCGSLMEAQNVFEGTQARDVISWNSMIAGHAQHGSYETAYKLFQEMQNEELEPDNITFASVLSGCKNPEALELGKQIHGRITESGLQLDVNLGNALINMYIRCGSLQDARNVFHSLQHRDVMSWTAMIGGCADQGEDMKAIELFWQMQNEGFRPVKSTFSSILKVCTSSACLDEGKKVIAYILNSGYELDTGVGNALISAYSKSGSMTDAREVFDKMPSRDIVSWNKIIAGYAQNGLGQTAVEFAYQMQEQDVVPNKFSFVSLLNACSSFSALEEGKRVHAEIVKRKLQGDVRVGAALISMYAKCGSQGEAQEVFDNIIEKNVVTWNAMINAYAQHGLASKALGFFNCMEKEGIKPDGSTFTSILSACNHAGLVLEGYQIFSSMESEYGVLPTIEHYGCLVGLLGRARRFQEAETLINQMPFPPDAAVWETLLGACRIHGNIALAEHAANNALKLNARNPAVYILLSNVYAAAGRWDDVAKIRRVMEGRGIRKEPGRSWIEVDNIIHEFIAADRSHPETAEIYAELKRLSVEMEEAGYFPDTQHVLHDLGKAHQETSLCTHSERLAIAYGLIKTPPGTPIRIFKNLRICGDCHTASKFISKLVGREIIARDSNRFHSFKNGKCSCEDYW

SEQ ID NO: 29 - protein, 1042 aa, Physcomitrella patens
MSLPWGLDFTTVSTAKQTQAGFSVLKKEKHSLRDDLILATSRQNDAGHKIDLVGDSARNFLVKQIARDSDTLVRADLKKECSRAYGKPSLLVEAHRSSSMHSQSRVQLCKWWFNTSGFRYSNGKPRVNKLYFHTYKDEKIMIEKDGAVDVVQYLQQQGAQVNSSDYMKMLKRCIEVKDLVAGRQVHQHIIQHRTVPDQYTVNALINMYIQCGSIEEARQVWKKLSYMERTVHSWNAMVVGYIQYGYIEKALKLLRQMQQHGLAPDRTTIMSFLSSCKSPGALEWGREIHFQAMQAGLLFDVKVANCILNMYAKCGSIEEAREVFDKMEKKSVVSWTITIGGYADCGRSETAFEIFQKMEQEGVVPNRITYISVLNAFSSPAALKWGKAVHSRILNAGHESDTAVGTALVKMYAKCGSYKDCRQVFEKLVNRDLIAWNTMIGGLAEGGYWEEASEVYNQMQREGVMPNKITYVILLNACVNSAALHWGKEIHSRVAKAGFTSDIGVQNALISMYSRCGSIKDARLVFDKMVRKDVISWTAMIGGLAKSGFGAEALTVYQEMQQAGVEPNRVTYTSILNACSSPAALEWGRRIHQQVVEAGLATDAHVGNTLVNMYSMCGSVKDARQVFDRMIQRDIVAYNAMIGGYAAHNLGKEALKLFDRLQEEGLKPDKVTYINMLNACANSGSLEWAREIHTLVRKGGFFSDTSVGNALVSTYAKCGSFSDASIVFEKMTKRNVISWNAIIGGSAQHGRGQDALQLFERMKMEGVKPDIVTFVSLLSACSHAGLLEEGRRYFCSMSQDFAIIPTIEHYGCMVDLLGRAGQLDEAEALIKTMPFQANTRIWGALLGACRIHGNVPVAERAAESSLKLDLDNAVVYVALSHMYAAAGMWDSAAKLRKLMEQRGVTKEPGRSWIQVGDKLHYFVAEDRSHPQSEKIYAELDRLTHAMKMKGYVPDTRSVMHDVDEGEKENAVCHHSERLAIAYGLISTPPGTRIHIFKNLRVCPDCHTATKFISKIVDREIIARDVNRFHHFKDGVCSCGDYW

SEQ ID NO: 30 - protein, 804 aa, Physcomitrella patens
MIPTGKDGWYAPADVLQYLHRKGPQVDSYDYVKLLQSCVKAKDLAVGKQVHEHILRCGVKPNVYITNTLLKLYAHCGSVNEARQLFDKFSNKSVVSWNVMISGYAHRGLAQEAFNLFTLMQQERLEPDKFTFVSILSACSSPAVLNWGREIHVRVMEAGLANDTTVGNALISMYAKCGSVRDARRVFDAMASRDEVSWTTLTGAYAESGYGEESLKTYHAMLQERVRPSRITYMNVLSACGSLAALEKGKQIHAHIVESEYHSDVRVSTALTKMYMKCGAFKDAREVFECLSYRDVIAWNTMIRGFVDSGQLEEAHGTFHRMLEEGVAPDRATYTTVLSACARPGGLARGKEIHARAAKDGLVSDVRFGNALINMYSKAGSMKDARQVFDRMPKRDVVSWTTLLGRYADCDQVVESFTTFKQMLQQGVKANKITYMCVLKACSNPVALKWGKEIHAEVVKAGLLADLAVTNALMSMYFKCGSVEDAIRVFEGMSMRDVVTWNTLIGGLGQNGRGLEALQRYEVMKSEGMRPNAATFVNVLSACRVCNLVEEGRRQFAFMSKDYGIVPTEKHYACMVDILARAGHLREAEDVILTIPLKPSAAMWGALLAACRIHCNVEIGERAAEHCLKLEPQNAGLYVSLSAIYAAAGMWRDVAKLRKFMKERGVKKEPGRSWIEIAGEVHSFVARDQSHPRTQEIYAELETLKKQMKSLGYVPDTRFVMHDLDDEGKERAVCHHSEKLAIAYGLISTPPGTPIRISKNLRVCTDCHTATKFISKITKREIIARDAHRFHHFKNGECSCGDYW

SEQ ID NO: 31 - protein, 890 aa, Physcomitrella patens
MLSRRFGLRRWKQVLQNYHVGAMTSIVYNDGFASTGKELDGPTSVSGGEVWRLCKAGRLREAIQLLGIIKQRGLLVNSNTYGCVIEHCAKARRFEDGKMVHKQLDELGVEIDIYLGNSLINFYSKFEDVASAEQVFRRMTLRDVVTWSSMIAAYAGNNHPAKAFDTFERMTDANIEPNRITFLSILKACNNYSILEKGRKIHTIVKAMGMETDVAVATALITMYSKCGEISVACEVFHKMTERNVVSWTAIIQANAQHRKLNEAFELYEQMLQAGISPNAVTFVSLLNSCNTPEALNRGRRIHSHISERGLETDMIVANALITMYCKCNSVQEAREIFDRMSKRDVISWSAMIAGYAQSGYKDKESIDEVFQLLERMRREGVFPNKVTFMSILRACTAHGALEQGRQIHAELSKVGFELDRSLQTAIFNMYAKCGSIYEAEQVFSKMANKNVVAWTSFLSMYIKCGDLSSAEKVFSEMPTRNVVSWNLMIAGYAQNGDIVKVFELLSSMKAEGFQPDRVTVITILEACGALAGLERGKLVHAEAVKLGLESDTVVATSLIGMYSKCGQVAEARTVFDKMSNRDTVAWNAMLAGYGQHGDGLEAVDLFKRMLKERVSPNEITLTAVISACSRAGLVQEGREIFRMMQEDFKMTPRKQHYGCMVDLLGRAGRLQEAEEFIQSMPCEPDISVWHALLGACKSHNNVQLAERAAHHILELEPSYASVYITLSNIYAQAGRWDDSTKVRRVMDDRGLKKDRGESSIEIDGRIHTFVAEDCAHPEIDAIHAELETLTKEMKEAGYTPDMRFVLHDVDDVQKEKALCHHSEKLAIAYGLLKTPSGTPIRIMKNLRVCGDCHTATKFISKIRKREIVARDANRFHYFNNGTCSCGDFW

SEQ ID NO: 32 - RNA, 31 nt, Physcomitrella patens
  source 1..31: mol_type = genomic RNA
ctataggtattggaaacgtatttagttcttc

SEQ ID NO: 33 - RNA, 31 nt, Physcomitrella patens
  source 1..31: mol_type = other RNA
ctatttcaatggttggtaagtagagatgttc

SEQ ID NO: 34 - RNA, 31 nt, Physcomitrella patens
  source 1..31: mol_type = other RNA
gtagagatgtttccacaggtgctcctttttc

SEQ ID NO: 35 - RNA, 31 nt, Physcomitrella patens
  source 1..31: mol_type = other RNA
atcccgatcctgaggtctatattctaatttc

SEQ ID NO: 36 - RNA, 31 nt, Physcomitrella patens
  source 1..31: mol_type = other RNA
acaattactatcaaagctattggacatcaac

SEQ ID NO: 37 - RNA, 31 nt, Physcomitrella patens
  source 1..31: mol_type = other RNA
cactttggctttgaagcagctgcttggtacc

SEQ ID NO: 38 - RNA, 31 nt, Physcomitrella patens
  source 1..31: mol_type = other RNA
ttattatatttgatttggaagtcaccttttc

SEQ ID NO: 39 - RNA, 31 nt, Physcomitrella patens
  source 1..31: mol_type = other RNA
atttttatataggtatagacggtatctcttc

SEQ ID NO: 40 - RNA, 31 nt, Physcomitrella patens
  source 1..31: mol_type = other RNA
caaacagtagacttttcgactatttttgctc

SEQ ID NO: 41 - RNA, 31 nt, Physcomitrella patens
  source 1..31: mol_type = other RNA
ggaaaatctgcacaaataggattgcatactc

SEQ ID NO: 42 - RNA, 31 nt, Physcomitrella patens
  source 1..31: mol_type = other RNA
ctaaaacaaatattttaataataaaaaaatc

SEQ ID NO: 43 - RNA, 31 nt, Physcomitrella patens
  source 1..31: mol_type = other RNA
aaaacaaatattttaataataaaaaaatcac

SEQ ID NO: 44 - RNA, 31 nt, Physcomitrella patens
  source 1..31: mol_type = other RNA
atcaaatacgttatgaatattttttaaagtc

SEQ ID NO: 45 - RNA, 31 nt, Arabidopsis thaliana
  source 1..31: mol_type = other RNA
aaatgagtagttcagaaagaatcgagctttc

SEQ ID NO: 46 - RNA, 31 nt, Arabidopsis thaliana
  source 1..31: mol_type = other RNA
ttttttttatcgatattcttgtttactactc

SEQ ID NO: 47 - RNA, 31 nt, Arabidopsis thaliana
  source 1..31: mol_type = other RNA
gatttaataccgatattttagcaacaaatcc

SEQ ID NO: 48 - RNA, 31 nt, Arabidopsis thaliana
  source 1..31: mol_type = other RNA
gtttttatgtcagcaacagaagcccaagctc

SEQ ID NO: 49 - RNA, 31 nt, Arabidopsis thaliana
  source 1..31: mol_type = other RNA
ccaagatttttcttgttcttatataattctc

SEQ ID NO: 50 - RNA, 31 nt, Arabidopsis thaliana
  source 1..31: mol_type = other RNA
tgattgtatgtgtgatagcatctactatacc

SEQ ID NO: 51 - RNA, 31 nt, Arabidopsis thaliana
  source 1..31: mol_type = other RNA
cctccactagcaggtttttttggaaaactcc

SEQ ID NO: 52 - RNA, 31 nt, Arabidopsis thaliana
  source 1..31: mol_type = other RNA
ctcgaattttcgatattcctttttatttctc

SEQ ID NO: 53 - RNA, 31 nt, Arabidopsis thaliana
  source 1..31: mol_type = other RNA
ctgttacttcgaaagtagctgcttcagcttc

SEQ ID NO: 54 - RNA, 31 nt, Arabidopsis thaliana
  source 1..31: mol_type = other RNA
ttctttctgttacttcgaaagtagctgcttc

SEQ ID NO: 55 - RNA, 31 nt, Arabidopsis thaliana
  source 1..31: mol_type = other RNA
gaattgggttcaagctttccctagccccttc

SEQ ID NO: 56 - RNA, 31 nt, Arabidopsis thaliana
  source 1..31: mol_type = other RNA
atgggtggggcaagctcttctattctggttc

SEQ ID NO: 57 - RNA, 31 nt, Arabidopsis thaliana
  source 1..31: mol_type = other RNA
ctaacgatttaataactatctttgtagctcc

SEQ ID NO: 58 - RNA, 31 nt, Arabidopsis thaliana
  source 1..31: mol_type = other RNA
ttggcctaattcttcttctgatgatcgattc

SEQ ID NO: 59 - RNA, 31 nt, Arabidopsis thaliana
  source 1..31: mol_type = other RNA
tccaaataatttatgcagcttcaacatctcc

SEQ ID NO: 60 - RNA, 31 nt, Arabidopsis thaliana
  source 1..31: mol_type = other RNA
tcggtacaatccaaataatttatgcagcttc

SEQ ID NO: 61 - RNA, 31 nt, Arabidopsis thaliana
  source 1..31: mol_type = other RNA
ttggatttcttattgcttttgccgtcaaatc

SEQ ID NO: 62 - RNA, 31 nt, Arabidopsis thaliana
  source 1..31: mol_type = other RNA
tgttagcaatgtacagcggtcaaataggatc

SEQ ID NO: 63 - RNA, 31 nt, Arabidopsis thaliana
  source 1..31: mol_type = other RNA
ctttggacctggtgtatcttgtctttaccac

SEQ ID NO: 64 - RNA, 31 nt, Arabidopsis thaliana
  source 1..31: mol_type = other RNA
actttatcgatccacttacttctattatgtc

SEQ ID NO: 65 - RNA, 31 nt, Arabidopsis thaliana
  source 1..31: mol_type = other RNA
tacatgattttcttttagtttttctgggatc

SEQ ID NO: 66 - RNA, 31 nt, Arabidopsis thaliana
  source 1..31: mol_type = other RNA
aaaaacatattttattgagtcccttcatgcc

SEQ ID NO: 67 - RNA, 31 nt, Arabidopsis thaliana
  source 1..31: mol_type = other RNA
ggcattccattaataacaggccgttttgatc

SEQ ID NO: 68 - RNA, 31 nt, Arabidopsis thaliana
  source 1..31: mol_type = other RNA
ctgttcatggactagctgtacctaccgtttc

SEQ ID NO: 69 - RNA, 31 nt, Arabidopsis thaliana
  source 1..31: mol_type = other RNA
tggcagtttttgcattaattattacttcatc

SEQ ID NO: 70 - RNA, 31 nt, Arabidopsis thaliana
  source 1..31: mol_type = other RNA
atcaatatacttttaatgtcgaatcaggatc

SEQ ID NO: 71 - RNA, 31 nt, Arabidopsis thaliana
  source 1..31: mol_type = other RNA
tagaaggaacatgtattacacgtgcaaaatc

SEQ ID NO: 72 - RNA, 31 nt, Arabidopsis thaliana
  source 1..31: mol_type = other RNA
ataacccagaaataattcgtgtatatatttc

SEQ ID NO: 73 - RNA, 31 nt, Arabidopsis thaliana
  source 1..31: mol_type = other RNA
ggaaacaaaaaatatctattctagttctatc

SEQ ID NO: 74 - RNA, 31 nt, Arabidopsis thaliana
  source 1..31: mol_type = other RNA
acattcctttaatgaattcccttggaacttc

SEQ ID NO: 75 - RNA, 31 nt, Arabidopsis thaliana
  source 1..31: mol_type = other RNA
aaaaacctactttcttacgattacgaggttc

SEQ ID NO: 76 - RNA, 31 nt, Arabidopsis thaliana
  source 1..31: mol_type = other RNA
gacataaaaaggaaattctttttgaaatatc

SEQ ID NO: 77 - RNA, 31 nt, Arabidopsis thaliana
  source 1..31: mol_type = other RNA
aatggaaaattcatggaaaattacaatcccc

SEQ ID NO: 78 - RNA, 31 nt, Arabidopsis thaliana
  source 1..31: mol_type = other RNA
aaaaaaaatatcatttgattcgtcgatcctc

SEQ ID NO: 79 - RNA, 31 nt, Arabidopsis thaliana
  source 1..31: mol_type = other RNA
ttatcgcggaattagacctgctattaacgtc

SEQ ID NO: 80 - RNA, 31 nt, Arabidopsis thaliana
  source 1..31: mol_type = other RNA
tgaaagctatgaaacaagtatgcggtagttc

SEQ ID NO: 81 - RNA, 31 nt, Arabidopsis thaliana
  source 1..31: mol_type = other RNA
tcaatagaggtgcaaggctgacagaagtacc

SEQ ID NO: 82 - RNA, 31 nt, Arabidopsis thaliana
  source 1..31: mol_type = other RNA
acagaatctctcaatatgagaaagccattcc

SEQ ID NO: 83 - RNA, 31 nt, Arabidopsis thaliana
  source 1..31: mol_type = other RNA
gtggattaactaacgaaagaaaaatggaacc

SEQ ID NO: 84 - RNA, 31 nt, Arabidopsis thaliana
  source 1..31: mol_type = other RNA
atcggaaacttctatttctcattcacaaatc

SEQ ID NO: 85 - RNA, 31 nt, Arabidopsis thaliana
  source 1..31: mol_type = other RNA
tggcgcgaaatccatcattggctaaacaatc

SEQ ID NO: 86 - RNA, 31 nt, Arabidopsis thaliana
  source 1..31: mol_type = other RNA
ctctaaccgaagctattgcattgtttgcccc

SEQ ID NO: 87 - RNA, 31 nt, Arabidopsis thaliana
  source 1..31: mol_type = other RNA
ctcaacccgagatgttagaaggtgcaaaatc

SEQ ID NO: 88 - RNA, 31 nt, Arabidopsis thaliana
  source 1..31: mol_type = other RNA
caataggtgccggagctgctacaattgcttc

SEQ ID NO: 89 - RNA, 31 nt, Arabidopsis thaliana
  source 1..31: mol_type = other RNA
ccttaatgctaggttttgaaaaagacttttc

SEQ ID NO: 90 - RNA, 31 nt, Arabidopsis thaliana
  source 1..31: mol_type = other RNA
taggttttgaaaaagacttttcatgtcattc

SEQ ID NO: 91 - RNA, 31 nt, Arabidopsis thaliana
  source 1..31: mol_type = other RNA
aaagacttttcatgtcattcccatttaggtc

SEQ ID NO: 92 - RNA, 31 nt, Arabidopsis thaliana
  source 1..31: mol_type = other RNA
aagacttttcatgtcattcccatttaggtcc

SEQ ID NO: 93 - RNA, 31 nt, Arabidopsis thaliana
  source 1..31: mol_type = other RNA
ttttcatgtcattcccatttaggtccgattc

SEQ ID NO: 94 - RNA, 31 nt, Arabidopsis thaliana
  source 1..31: mol_type = other RNA
atgtcattcccatttaggtccgattcggatc

SEQ ID NO: 95 - RNA, 31 nt, Arabidopsis thaliana
  source 1..31: mol_type = other RNA
agaagaagtaaggaaatgagacgactttttc

SEQ ID NO: 96 - RNA, 31 nt, Arabidopsis thaliana
  source 1..31: mol_type = other RNA
tgtcattcccatttaggtccgattcggatcc

SEQ ID NO: 97 - RNA, 31 nt, Arabidopsis thaliana
  source 1..31: mol_type = other RNA
attcccatttaggtccgattcggatccctcc

SEQ ID NO: 98 - RNA, 31 nt, Arabidopsis thaliana
  source 1..31: mol_type = other RNA
ttaggtccgattcggatccctccgttgtttc

SEQ ID NO: 99 - RNA, 31 nt, Arabidopsis thaliana
  source 1..31: mol_type = other RNA
cgattcggatccctccgttgtttccttttcc

SEQ ID NO: 100 - RNA, 31 nt, Arabidopsis thaliana
  source 1..31: mol_type = other RNA
attcggatccctccgttgtttccttttcctc

SEQ ID NO: 101 - RNA, 31 nt, Arabidopsis thaliana
  source 1..31: mol_type = other RNA
tccctccgttgtttccttttcctcctgcacc

SEQ ID NO: 102 - RNA, 31 nt, Arabidopsis thaliana
  source 1..31: mol_type = other RNA
ccgttgtttccttttcctcctgcaccttttc

SEQ ID NO: 103 - RNA, 31 nt, Arabidopsis thaliana
  source 1..31: mol_type = other RNA
cgttgtttccttttcctcctgcaccttttcc

SEQ ID NO: 104 - RNA, 31 nt, Arabidopsis thaliana
  source 1..31: mol_type = other RNA
gaaatgagacgactttttcttgaactatatc

SEQ ID NO: 105 - RNA, 31 nt, Arabidopsis thaliana
  source 1..31: mol_type = other RNA
aagatcctacttctacaattggtgggtcacc

SEQ ID NO: 106 - RNA, 31 nt, Arabidopsis thaliana
  source 1..31: mol_type = other RNA
ttggtgggtcaccgggttattcaaataagtc

SEQ ID NO: 107 - RNA, 31 nt, Arabidopsis thaliana
  source 1..31: mol_type = other RNA
ttttctgtggttttcccatgttacaactttc

SEQ ID NO: 108 - RNA, 31 nt, Arabidopsis thaliana
  source 1..31: mol_type = other RNA
tcgtaccaattcggtcgatccggaatggatc

SEQ ID NO: 109 - RNA, 31 nt, Arabidopsis thaliana
  source 1..31: mol_type = other RNA
ggtcgatccggaatggatcggttaaacattc

SEQ ID NO: 110 - RNA, 31 nt, Arabidopsis thaliana
  source 1..31: mol_type = other RNA
gtcgatccggaatggatcggttaaacattcc

SEQ ID NO: 111 - RNA, 31 nt, Arabidopsis thaliana
  source 1..31: mol_type = other RNA
attccattagggagcctggtcttgactcttc

SEQ ID NO: 112 - RNA, 31 nt, Arabidopsis thaliana
  source 1..31: mol_type = other RNA
gtcttgactcttctgtgtggtattcattctc

SEQ ID NO: 113 - RNA, 31 nt, Arabidopsis thaliana
  source 1..31: mol_type = other RNA
tgactcttctgtgtggtattcattctcgttc

SEQ ID NO: 114 - RNA, 31 nt, Arabidopsis thaliana
  source 1..31: mol_type = other RNA
gaatcacatccagcagtggttggaacagctc

SEQ ID NO: 115 - RNA, 31 nt, Arabidopsis thaliana
  source 1..31: mol_type = other RNA
tccagcagtggttggaacagctcgcaaaatc

SEQ ID NO: 116 - RNA, 31 nt, Arabidopsis thaliana
  source 1..31: mol_type = other RNA
ccagcagtggttggaacagctcgcaaaatcc

SEQ ID NO: 117 - RNA, 31 nt, Arabidopsis thaliana
  source 1..31: mol_type = other RNA
gttggaacagctcgcaaaatccaaccacttc

SEQ ID NO: 118 - RNA, 31 nt, Arabidopsis thaliana
  source 1..31: mol_type = other RNA
cttcacctactttattgcccctaaccgtttc

SEQ ID NO: 119 - RNA, 31 nt, Arabidopsis thaliana
  source 1..31: mol_type = other RNA
tcacctactttattgcccctaaccgtttctc

SEQ ID NO: 120 - RNA, 31 nt, Arabidopsis thaliana
  source 1..31: mol_type = other RNA
ctattgaaacagaatggtttcatgttctttc

SEQ ID NO: 121 - RNA, 31 nt, Arabidopsis thaliana
  source 1..31: mol_type = other RNA
ttgaaacagaatggtttcatgttctttcatc

SEQ ID NO: 122 - RNA, 31 nt, Arabidopsis thaliana
  source 1..31: mol_type = other RNA
ggtttcatgttctttcatcgattggttattc

SEQ ID NO: 123 - RNA, 31 nt, Arabidopsis thaliana
  source 1..31: mol_type = other RNA
ttcatgttctttcatcgattggttattcctc

SEQ ID NO: 124 - RNA, 31 nt, Arabidopsis thaliana
  source 1..31: mol_type = other RNA
tctcctccacaccaatcacgagtttttcttc

SEQ ID NO: 125 - RNA, 31 nt, Arabidopsis thaliana
  source 1..31: mol_type = other RNA
ctccacaccaatcacgagtttttcttcattc

SEQ ID NO: 126 - RNA, 31 nt, Arabidopsis thaliana
  source 1..31: mol_type = other RNA
cacaccaatcacgagtttttcttcattcctc

SEQ ID NO: 127 - RNA, 31 nt, Arabidopsis thaliana
  source 1..31: mol_type = other RNA
caccaatcacgagtttttcttcattcctctc

SEQ ID NO: 128 - RNA, 31 nt, Arabidopsis thaliana
  source 1..31: mol_type = other RNA
tcttggttgttcttaacagcgatggctattc

SEQ ID NO: 129 - RNA, 31 nt, Arabidopsis thaliana
  source 1..31: mol_type = other RNA
catttaagtcttggggtagcaccactagatc

SEQ ID NO: 130 - RNA, 31 nt, Arabidopsis thaliana
  source 1..31: mol_type = other RNA
attctcgtattctctatgtacatgttcctgc

SEQ ID NO: 131 - RNA, 31 nt, Arabidopsis thaliana
  source 1..31: mol_type = other RNA
cgtattctctatgtacatgttcctgcggctc

SEQ ID NO: 132 - RNA, 31 nt, Arabidopsis thaliana
  source 1..31: mol_type = other RNA
ttcctattaacaaaacatcccctttatcttc

SEQ ID NO: 133 - RNA, 31 nt, Arabidopsis thaliana
  source 1..31: mol_type = other RNA
acgttgtttaccttagttactggggggtttc

SEQ ID NO: 134 - RNA, 31 nt, Arabidopsis thaliana
  source 1..31: mol_type = other RNA
gggatgctcgtttgacctctgtattcatctc

SEQ ID NO: 135 - RNA, 31 nt, Arabidopsis thaliana
  source 1..31: mol_type = other RNA
gctcgtttgacctctgtattcatctcgtttc

SEQ ID NO: 136 - RNA, 31 nt, Arabidopsis thaliana
  source 1..31: mol_type = other RNA
atctcgtttcttatttacctgggtgcactgc

SEQ ID NO: 137 - RNA, 31 nt, Arabidopsis thaliana
  source 1..31: mol_type = other RNA
cactgcgttttcaaaagcttcctgtcgaacc

SEQ ID NO: 138 - RNA, 31 nt, Arabidopsis thaliana
  source 1..31: mol_type = other RNA
aaaagcttcctgtcgaaccggcttctatttc

SEQ ID NO: 139 - RNA, 31 nt, Arabidopsis thaliana
  source 1..31: mol_type = other RNA
cttcctgtcgaaccggcttctatttcaattc

SEQ ID NO: 140 - RNA, 31 nt, Arabidopsis thaliana
  source 1..31: mol_type = other RNA
ctgtcgaaccggcttctatttcaattcgtgc

SEQ ID NO: 141 - RNA, 31 nt, Arabidopsis thaliana
  source 1..31: mol_type = other RNA
aaccggcttctatttcaattcgtgctggacc

SEQ ID NO: 142 - RNA, 31 nt, Arabidopsis thaliana
  source 1..31: mol_type = other RNA
ggcttctatttcaattcgtgctggaccgatc

SEQ ID NO: 143 - RNA, 31 nt, Arabidopsis thaliana
  source 1..31: mol_type = other RNA
ctggaccgatcgatataccaataataaagtc

SEQ ID NO: 144 - RNA, 31 nt, Arabidopsis thaliana
  source 1..31: mol_type = other RNA
taaagtcttcagtcaactggtggaatacatc

SEQ ID NO: 145 - RNA, 31 nt, Arabidopsis thaliana
  source 1..31: mol_type = other RNA
catcgcatcaacctgggagcattagccgatc

SEQ ID NO: 146 - RNA, 31 nt, Arabidopsis thaliana
  source 1..31: mol_type = other RNA
attagccgatctggtacatcaatacatgttc

SEQ ID NO: 147 - RNA, 31 nt, Arabidopsis thaliana
  source 1..31: mol_type = other RNA
gatctggtacatcaatacatgttcctatgcc

SEQ ID NO: 148 - RNA, 31 nt, Arabidopsis thaliana
  source 1..31: mol_type = other RNA
ttccaatcttgtctaactttgctaacttccc

SEQ ID NO: 149 - RNA, 31 nt, Arabidopsis thaliana
  source 1..31: mol_type = other RNA
tcttgtctaactttgctaacttccccttctc

SEQ ID NO: 150 - RNA, 31 nt, Arabidopsis thaliana
  source 1..31: mol_type = other RNA
gtctaactttgctaacttccccttctcaacc

SEQ ID NO: 151 - RNA, 31 nt, Arabidopsis thaliana
  source 1..31: mol_type = other RNA
tctaactttgctaacttccccttctcaaccc

SEQ ID NO: 152 - RNA, 31 nt, Arabidopsis thaliana
  source 1..31: mol_type = other RNA
ctttgctaacttccccttctcaacccgtatc

SEQ ID NO: 153 - RNA, 31 nt, Arabidopsis thaliana
  source 1..31: mol_type = other RNA
gtatcttgtttgttctggaaacacgtcttcc

SEQ ID NO: 154 - RNA, 31 nt, Arabidopsis thaliana
  source 1..31: mol_type = other RNA
tgtttgttctggaaacacgtcttcctattcc

SEQ ID NO: 155 - RNA, 31 nt, Arabidopsis thaliana
  source 1..31: mol_type = other RNA
cgtcttcctattccatcttttctcgaatctc

SEQ ID NO: 156 - RNA, 31 nt, Arabidopsis thaliana
  source 1..31: mol_type = other RNA
ctattgaaatggttcgtcagtagagatgttc

SEQ ID NO: 157 - RNA, 31 nt, Arabidopsis thaliana
  source 1..31: mol_type = other RNA
agcacggctgaagtggctatacatacaaatc

SEQ ID NO: 158 - RNA, 31 nt, Arabidopsis thaliana
  source 1..31: mol_type = other RNA
atacaaatctatttacggatctatatgcttc

SEQ ID NO: 159 - RNA, 31 nt, Arabidopsis thaliana
  source 1..31: mol_type = other RNA
aagttccagaacaggaggttggtataccacc

SEQ ID NO: 160 - RNA, 31 nt, Arabidopsis thaliana
  source 1..31: mol_type = other RNA
gtagagatgttcccacgggtgcccctttttc

SEQ ID NO: 161 - RNA, 31 nt, Arabidopsis thaliana
  source 1..31: mol_type = other RNA
tagagatgttcccacgggtgcccctttttcc

SEQ ID NO: 162 - RNA, 31 nt, Arabidopsis thaliana
  source 1..31: mol_type = other RNA
ataatgaaactgccttttattttttttattc

SEQ ID NO: 163 - RNA, 31 nt, Arabidopsis thaliana
  source 1..31: mol_type = other RNA
taggatttatgttggcttcgttgggaggctc

SEQ ID NO: 164 - RNA, 31 nt, Arabidopsis thaliana
  source 1..31: mol_type = other RNA
cagctccaaaaggataagttgcgttggaatc

SEQ ID NO: 165 - RNA, 31 nt, Arabidopsis thaliana
  source 1..31: mol_type = other RNA
ctttttccaatggtactataattcctattcc

SEQ ID NO: 166 - RNA, 31 nt, Arabidopsis thaliana
  source 1..31: mol_type = other RNA
atggtactataattcctattcctatctcttc

SEQ ID NO: 167 - RNA, 31 nt, Arabidopsis thaliana
  source 1..31: mol_type = other RNA
actataattcctattcctatctcttcattcc

SEQ ID NO: 168 - RNA, 31 nt, Arabidopsis thaliana
  source 1..31: mol_type = other RNA
cctatctcttcattccctcttttggtctatc

SEQ ID NO: 169 - RNA, 31 nt, Arabidopsis thaliana
  source 1..31: mol_type = other RNA
cttattttttttcgttcccgttcttcatttc

SEQ ID NO: 170 - RNA, 31 nt, Arabidopsis thaliana
  source 1..31: mol_type = other RNA
ttattttttttcgttcccgttcttcatttcc

SEQ ID NO: 171 - RNA, 31 nt, Arabidopsis thaliana
  source 1..31: mol_type = other RNA
caagggggacttctcatatttagaatctttc

SEQ ID NO: 172 - RNA, 31 nt, Arabidopsis thaliana
  source 1..31: mol_type = other RNA
ttctgcggtgtgctctgtttactattctttc

SEQ ID NO: 173 - RNA, 31 nt, Arabidopsis thaliana
  source 1..31: mol_type = other RNA
gtgctctgtttactattctttcgtactttcc

SEQ ID NO: 174 - RNA, 31 nt, Arabidopsis thaliana
  source 1..31: mol_type = other RNA
ttttcttttttattatttttatggtcgtgcc

SEQ ID NO: 175 - RNA, 31 nt, Arabidopsis thaliana
  source 1..31: mol_type = other RNA
gcacccccatttagatttagaaagaagggtc

SEQ ID NO: 176 - RNA, 31 nt, Arabidopsis thaliana
  source 1..31: mol_type = other RNA
tccatttgtgggaatttgatgatctataaac

SEQ ID NO: 177 - RNA, 31 nt, Arabidopsis thaliana
  source 1..31: mol_type = other RNA
agaaacaaccaccagcgtttggtgcagcacc

SEQ ID NO: 178 - RNA, 31 nt, Arabidopsis thaliana
  source 1..31: mol_type = other RNA
ggtgcattcttctttctttccttggtctttc

SEQ ID NO: 179 - RNA, 31 nt, Arabidopsis thaliana
  source 1..31: mol_type = other RNA
tctttccttggtctttcgttctgtcatattc

SEQ ID NO: 180 - RNA, 31 nt, Arabidopsis thaliana
  source 1..31: mol_type = other RNA
ccaattacaacgtattaaccgctaatgcacc

SEQ ID NO: 181 - RNA, 31 nt, Arabidopsis thaliana
  source 1..31: mol_type = other RNA
aatcatgagggtagtattttattatggtgtc

SEQ ID NO: 182 - RNA, 31 nt, Arabidopsis thaliana
  source 1..31: mol_type = other RNA
agggtagtattttattatggtgtcggatccc

SEQ ID NO: 183 - RNA, 31 nt, Arabidopsis thaliana
  source 1..31: mol_type = other RNA
tgtcggatcccaaatttttatggattttttc

SEQ ID NO: 184 - RNA, 31 nt, Arabidopsis thaliana
  source 1..31: mol_type = other RNA
cagagccataatgtcttaaaacaaggaggcc

SEQ ID NO: 185 - RNA, 31 nt, Arabidopsis thaliana
  source 1..31: mol_type = other RNA
atagggaaagtcttttttttttttttgtctc

SEQ ID NO: 186 - RNA, 31 nt, Arabidopsis thaliana
  source 1..31: mol_type = other RNA
aagtcttttttttttttttgtctcgaacttc

SEQ ID NO: 187 - RNA, 31 nt, Arabidopsis thaliana
  source 1..31: mol_type = other RNA
acttcgtgaagaactcaattttatctctccc

SEQ ID NO: 188 - RNA, 31 nt, Arabidopsis thaliana
  source 1..31: mol_type = other RNA
tatatgaattctttcattattcgttatttcc

SEQ ID NO: 189 - RNA, 31 nt, Arabidopsis thaliana
  source 1..31: mol_type = other RNA
gttctacgaacccttgttgattctgaacttc

SEQ ID NO: 190 - RNA, 31 nt, Arabidopsis thaliana
  source 1..31: mol_type = other RNA
gaaaattaaaaatcctctggacgcttggcgc

SEQ ID NO: 191 - RNA, 31 nt, Arabidopsis thaliana
  source 1..31: mol_type = other RNA
aaattaaaaatcctctggacgcttggcgctc

SEQ ID NO: 192 - RNA, 31 nt, Arabidopsis thaliana
  source 1..31: mol_type = other RNA
ggagctcttggcattgctttgtttttctctc

SEQ ID NO: 193 - RNA, 31 nt, Arabidopsis thaliana
  source 1..31: mol_type = other RNA
gagctcttggcattgctttgtttttctctcc

SEQ ID NO: 194 - RNA, 31 nt, Arabidopsis thaliana
  source 1..31: mol_type = other RNA
gcattgctttgtttttctctcctttcctatc

SEQ ID NO: 195 - RNA, 31 nt, Arabidopsis thaliana
  source 1..31: mol_type = other RNA
tctttgtttgtaccgaaccgcttgcagaatc

SEQ ID NO: 196 - RNA, 31 nt, Arabidopsis thaliana
  source 1..31: mol_type = other RNA
ccgaaccgcttgcagaatcaaatcctgttcc

SEQ ID NO: 197 - RNA, 31 nt, Arabidopsis thaliana
  source 1..31: mol_type = other RNA
aatcaaatcctgttccacaagatcctatatc

SEQ ID NO: 198 - RNA, 31 nt, Arabidopsis thaliana
  source 1..31: mol_type = other RNA
gatgccgccgaaaagaatggaacgctgcttc

SEQ ID NO: 199 - RNA, 31 nt, Arabidopsis thaliana
  source 1..31: mol_type = other RNA
gttggtggtttttaaccgtaggcatcttgcc

SEQ ID NO: 200 - RNA, 31 nt, Arabidopsis thaliana
  source 1..31: mol_type = other RNA
ggaagttggtgggcttatcatgaattaggtc

SEQ ID NO: 201 - RNA, 31 nt, Arabidopsis thaliana
  source 1..31: mol_type = other RNA
catgaattaggtcggggtggctggtggtttc

SEQ ID NO: 202 - RNA, 31 nt, Arabidopsis thaliana
  source 1..31: mol_type = other RNA
gatcccgtagaaaatgcttcttttatgcctc

SEQ ID NO: 203 - RNA, 31 nt, Arabidopsis thaliana
  source 1..31: mol_type = other RNA
tcttttatgcctcgggtattagccacagctc

SEQ ID NO: 204 - RNA, 31 nt, Arabidopsis thaliana
  source 1..31: mol_type = other RNA
acagctcgtattcattctgtaattttacccc

SEQ ID NO: 205 - RNA, 31 nt, Arabidopsis thaliana
  source 1..31: mol_type = other RNA
taattttaccccttcttcattcttggacctc

SEQ ID NO: 206 - RNA, 31 nt, Arabidopsis thaliana
  source 1..31: mol_type = other RNA
ggacctcgtttcttaatattgtgacttttcc

SEQ ID NO: 207 - RNA, 31 nt, Arabidopsis thaliana
  source 1..31: mol_type = other RNA
ttaatattgtgacttttccatgctgtgtctc

SEQ ID NO: 208 - RNA, 31 nt, Arabidopsis thaliana
  source 1..31: mol_type = other RNA
accttttcaatacggtccggattgctagctc

SEQ ID NO: 209 - RNA, 31 nt, Arabidopsis thaliana
  source 1..31: mol_type = other RNA
ggtggttcttccttctaatgaccggcatatc

SEQ ID NO: 210 - RNA, 31 nt, Arabidopsis thaliana
  source 1..31: mol_type = other RNA
ttcgtaatggaaagaaagagaccactacttc

SEQ ID NO: 211 - RNA, 31 nt, Arabidopsis thaliana
  source 1..31: mol_type = other RNA
agacttacatcacgatatctttttcttcctc

SEQ ID NO: 212 - RNA, 31 nt, Arabidopsis thaliana
  source 1..31: mol_type = other RNA
tttgaaaatgattgttctaaaatggttattc

SEQ ID NO: 213 - RNA, 31 nt, Arabidopsis thaliana
  source 1..31: mol_type = other RNA
ttgaaaatgattgttctaaaatggttattcc

SEQ ID NO: 214 - RNA, 31 nt, Arabidopsis thaliana
  source 1..31: mol_type = other RNA
attgttcatggaactactatcgagattcttc

SEQ ID NO: 215 - RNA, 31 nt, Arabidopsis thaliana
  source 1..31: mol_type = other RNA
tggaactactatcgagattcttcggaccatc

SEQ ID NO: 216 - RNA, 31 nt, Arabidopsis thaliana
  source 1..31: mol_type = other RNA
gaaaatgattgttctaaaatggttattcctc

SEQ ID NO: 217 - RNA, 31 nt, Arabidopsis thaliana
  source 1..31: mol_type = other RNA
ttcttcggaccatctttcctagtatcatctc

SEQ ID NO: 218 - RNA, 31 nt, Arabidopsis thaliana
  source 1..31: mol_type = other RNA
actatcaaagctattggacatcaatggtatc

SEQ ID NO: 219 - RNA, 31 nt, Arabidopsis thaliana
  source 1..31: mol_type = other RNA
ttccagaagaagatctagaattgggtcaatc

SEQ ID NO: 220 - RNA, 31 nt, Arabidopsis thaliana
  source 1..31: mol_type = other RNA
tacgtattattgtaacatctgctgatgtacc

SEQ ID NO: 221 - RNA, 31 nt, Arabidopsis thaliana
  source 1..31: mol_type = other RNA
atgtacctcatagttgggctgtaccttcctc

SEQ ID NO: 222 - RNA, 31 nt, Arabidopsis thaliana
  source 1..31: mol_type = other RNA
gtgagatttgtggaactaatcatgcctttac

SEQ ID NO: 223 - RNA, 31 nt, Arabidopsis thaliana
  source 1..31: mol_type = other RNA
gtgatgcagcggaaccatggcaattaggatc

SEQ ID NO: 224 - RNA, 31 nt, Arabidopsis thaliana
  source 1..31: mol_type = other RNA
gcctttacgtctatcgtcgtagaagctgttc

SEQ ID NO: 225 - RNA, 31 nt, Arabidopsis thaliana
  source 1..31: mol_type = other RNA
gaagctgttcctaggaaagattatggttctc

SEQ ID NO: 226 - RNA, 31 nt, Arabidopsis thaliana
  source 1..31: mol_type = other RNA
accaccgtaggaggtgtgatgtacatgcacc

SEQ ID NO: 227 - RNA, 31 nt, Arabidopsis thaliana
  source 1..31: mol_type = other RNA
gacatcataccaaagtcgtacaattaggacc

SEQ ID NO: 228 - RNA, 31 nt, Arabidopsis thaliana
  source 1..31: mol_type = other RNA
aagtcgtacaattaggacctcgatatggttc

SEQ ID NO: 229 - RNA, 31 nt, Arabidopsis thaliana
  source 1..31: mol_type = other RNA
ttatgttcttttttgcttttttttgggcttc

SEQ ID NO: 230 - RNA, 31 nt, Arabidopsis thaliana
  source 1..31: mol_type = other RNA
tgttcttttttgcttttttttgggcttcttc

SEQ ID NO: 231 - RNA, 31 nt, Arabidopsis thaliana
  source 1..31: mol_type = other RNA
atccttgggaaatcccttttcttaatacccc

SEQ ID NO: 232 - RNA, 31 nt, Arabidopsis thaliana
  source 1..31: mol_type = other RNA
aaatcccttttcttaatacccctattctccc

SEQ ID NO: 233 - RNA, 31 nt, Arabidopsis thaliana
  source 1..31: mol_type = other RNA
ttcggatagtatttatggttctacctttttc

SEQ ID NO: 234 - RNA, 31 nt, Arabidopsis thaliana
  source 1..31: mol_type = other RNA
gctccatttgttactattggacaaatttctc

SEQ ID NO: 235 - RNA, 31 nt, Arabidopsis thaliana
  source 1..31: mol_type = other RNA
agcaatcttagttattggtgggggttcggtc

SEQ ID NO: 236 - RNA, 31 nt, Arabidopsis thaliana
  source 1..31: mol_type = other RNA
tatatgcatgctaatggggcaagtatgtttc

SEQ ID NO: 237 - RNA, 31 nt, Arabidopsis thaliana
  source 1..31: mol_type = other RNA
gtttaccttcatatttttcgtggtctatatc

SEQ ID NO: 238 - RNA, 31 nt, Arabidopsis thaliana
  source 1..31: mol_type = other RNA
gccaccttaaatcgtttttttagtcttcatc

SEQ ID NO: 239 - RNA, 31 nt, Arabidopsis thaliana
  source 1..31: mol_type = other RNA
tttattttagtaggcgccagtcttcttcatc

SEQ ID NO: 240 - RNA, 31 nt, Arabidopsis thaliana
  source 1..31: mol_type = other RNA
attgtgccggaatggtatttcctaccgatcc

SEQ ID NO: 241 - RNA, 31 nt, Arabidopsis thaliana
  source 1..31: mol_type = other RNA
acaaagcgggaggtgtagccgcaatagcacc

SEQ ID NO: 242 - RNA, 31 nt, Arabidopsis thaliana
  source 1..31: mol_type = other RNA
agccgcaatagcaccagtttttatatgtctc

SEQ ID NO: 243 - RNA, 31 nt, Arabidopsis thaliana
  source 1..31: mol_type = other RNA
tatgtgcgtagttcaagttttcgaccgattc

SEQ ID NO: 244 - RNA, 31 nt, Arabidopsis thaliana
  source 1..31: mol_type = other RNA
acactgggcgggatctatcagcagcgaattc

SEQ ID NO: 245 - RNA, 31 nt, Arabidopsis thaliana
  source 1..31: mol_type = other RNA
ctgggcgggatctatcagcagcgaattcccc

SEQ ID NO: 246 - RNA, 31 nt, Arabidopsis thaliana
  source 1..31: mol_type = other RNA
gaggtatcattagccgaagaagaccctggcc

SEQ ID NO: 247 - RNA, 31 nt, Arabidopsis thaliana
  source 1..31: mol_type = other RNA
acgtcagcgacgaagacatcgtaaattggtc

SEQ ID NO: 248 - RNA, 31 nt, Arabidopsis thaliana
  source 1..31: mol_type = other RNA
cgtcagcgacgaagacatcgtaaattggtcc

SEQ ID NO: 249 - RNA, 31 nt, Arabidopsis thaliana
  source 1..31: mol_type = other RNA
taaattggtccgcgggcatcgcgataagtcc

SEQ ID NO: 250 - RNA, 31 nt, Arabidopsis thaliana
  source 1..31: mol_type = other RNA
gcgataagtcctctgtcctactacaggtgcc

SEQ ID NO: 251 - RNA, 31 nt, Arabidopsis thaliana
  source 1..31: mol_type = other RNA
aacctttatcaagtccgaacgattgtcgacc

SEQ ID NO: 252 - RNA, 31 nt, Arabidopsis thaliana
  source 1..31: mol_type = other RNA
cgaatataatcctcaagtactccaaagactc

SEQ ID NO: 253 - RNA, 31 nt, Arabidopsis thaliana
  source 1..31: mol_type = other RNA
tttcacaccatcgaccgacatcgactcatcc

SEQ ID NO: 254 - RNA, 31 nt, Arabidopsis thaliana
  source 1..31: mol_type = other RNA
aagagatcgacgatcccaagttcttttactc

SEQ ID NO: 255 - RNA, 31 nt, Arabidopsis thaliana
  source 1..31: mol_type = other RNA
tcccacacagtgtactactatcggccctacc

SEQ ID NO: 256 - RNA, 31 nt, Arabidopsis thaliana
  source 1..31: mol_type = other RNA
acgaaattccgattgttcagagagtcagatc

SEQ ID NO: 257 - RNA, 31 nt, Arabidopsis thaliana
  source 1..31: mol_type = other RNA
tgaaagaggcgatcagaatggtactcgaatc

SEQ ID NO: 258 - RNA, 31 nt, Arabidopsis thaliana
  source 1..31: mol_type = other RNA
tcggattgttacaacctctagcagatggttc

SEQ ID NO: 259 - RNA, 31 nt, Arabidopsis thaliana
  source 1..31: mol_type = other RNA
ccagatcatgagtaaataaaaaatcgaaaac

SEQ ID NO: 260 - RNA, 31 nt, Arabidopsis thaliana
  source 1..31: mol_type = other RNA
gtggctacatttatgttaagtctggtcgctc

SEQ ID NO: 261 - RNA, 31 nt, Arabidopsis thaliana
  source 1..31: mol_type = other RNA
ccttttgattatggtatggtattgtcagatc

SEQ ID NO: 262 - RNA, 31 nt, Arabidopsis thaliana
  source 1..31: mol_type = other RNA
cttttgattatggtatggtattgtcagatcc

SEQ ID NO: 263 - RNA, 31 nt, Arabidopsis thaliana
  source 1..31: mol_type = other RNA
ctaggtgtttatggaattattatagcgggtc

SEQ ID NO: 264 - RNA, 31 nt, Arabidopsis thaliana
  source 1..31: mol_type = other RNA
attcttattactgtactaatatgtgtaggtc

SEQ ID NO: 265 - RNA, 31 nt, Arabidopsis thaliana
  source 1..31: mol_type = other RNA
tcttattactgtactaatatgtgtaggtccc

SEQ ID NO: 266 - RNA, 31 nt, Arabidopsis thaliana
  source 1..31: mol_type = other RNA
cttattactgtactaatatgtgtaggtcccc

SEQ ID NO: 267 - RNA, 31 nt, Arabidopsis thaliana
  source 1..31: mol_type = other RNA
ctgtactaatatgtgtaggtccccgtaattc

SEQ ID NO: 268 - RNA, 31 nt, Arabidopsis thaliana
  source 1..31: mol_type = other RNA
agattgtcatggcgcaaaagcagatatggtc

SEQ ID NO: 269 - RNA, 31 nt, Arabidopsis thaliana
  source 1..31: mol_type = other RNA
ggcgcaaaagcagatatggtctggtattccc

SEQ ID NO: 270 - RNA, 31 nt, Arabidopsis thaliana
  source 1..31: mol_type = other RNA
attcccctgttccctgtattggttatgttcc

SEQ ID NO: 271 - RNA, 31 nt, Arabidopsis thaliana
  source 1..31: mol_type = other RNA
ttccctgtattggttatgttccttatttctc

SEQ ID NO: 272 - RNA, 31 nt, Arabidopsis thaliana
  source 1..31: mol_type = other RNA
cgtttgatctcccagaagcggaagctgaatc

SEQ ID NO: 273 - RNA, 31 nt, Arabidopsis thaliana
  source 1..31: mol_type = other RNA
gctataatgtagaatattcttcaatggggtc

SEQ ID NO: 274 - RNA, 31 nt, Arabidopsis thaliana
  source 1..31: mol_type = other RNA
agtatgccaatatgatcttaatgagtggtcc

SEQ ID NO: 275 - RNA, 31 nt, Arabidopsis thaliana
  source 1..31: mol_type = other RNA
taatgagtggtccatgcacattgttctttcc

SEQ ID NO: 276 - RNA, 31 nt, Arabidopsis thaliana
  source 1..31: mol_type = other RNA
catgcacattgttctttccaggaggttggcc

SEQ ID NO: 277 - RNA, 31 nt, Arabidopsis thaliana
  source 1..31: mol_type = other RNA
ttgttctttccaggaggttggccgcctatcc

SEQ ID NO: 278 - RNA, 31 nt, Arabidopsis thaliana
  source 1..31: mol_type = other RNA
atcttcccattttcaagaagatcccgggctc

SEQ ID NO: 279 - RNA, 31 nt, Arabidopsis thaliana
  source 1..31: mol_type = other RNA
ggctcgatctggtttagtatcaaggttcttc

SEQ ID NO: 280 - RNA, 31 nt, Arabidopsis thaliana
  source 1..31: mol_type = other RNA
tatcgttatgatcaattaatgggacttggcc

SEQ ID NO: 281 - RNA, 31 nt, Arabidopsis thaliana
  source 1..31: mol_type = other RNA
cggaaagtgttcttgcctctatcattagctc

SEQ ID NO: 282 - RNA, 31 nt, Arabidopsis thaliana
  source 1..31: mol_type = other RNA
ttcttgcctctatcattagctcgggtagtcc

SEQ ID NO: 283 - RNA, 31 nt, Arabidopsis thaliana
  source 1..31: mol_type = other RNA
taatgacgatggatgcattcgccatagtttc

SEQ ID NO: 284 - RNA, 31 nt, Arabidopsis thaliana
  source 1..31: mol_type = other RNA
tgggcgctctagccaaaacgaatcctatttc

SEQ ID NO: 285 - RNA, 31 nt, Arabidopsis thaliana
  source 1..31: mol_type = other RNA
acccccgttagccggcttttgtagcaaattc

SEQ ID NO: 286 - RNA, 31 nt, Arabidopsis thaliana
  source 1..31: mol_type = other RNA
gctttgggttgtggggcttactttctagccc

SEQ ID NO: 287 - RNA, 31 nt, Arabidopsis thaliana
  source 1..31: mol_type = other RNA
ctttgggttgtggggcttactttctagcccc

SEQ ID NO: 288 - RNA, 31 nt, Arabidopsis thaliana
  source 1..31: mol_type = other RNA
ccagtgggagtagtgactagcgttataggtc

SEQ ID NO: 289 - RNA, 31 nt, Arabidopsis thaliana
  source 1..31: mol_type = other RNA
tactagcaatgacttcctttttcattacttc

SEQ ID NO: 290 - RNA, 31 nt, Arabidopsis thaliana
  source 1..31: mol_type = other RNA
tagcaatgacttcctttttcattacttcatc

SEQ ID NO: 291 - RNA, 31 nt, Arabidopsis thaliana
  source 1..31: mol_type = other RNA
tctcagttactcatcaaatggcactcagttc

SEQ ID NO: 292 - RNA, 31 nt, Arabidopsis thaliana
  source 1..31: mol_type = other RNA
tctttttaggagggacaattttacatatttc

SEQ ID NO: 293 - RNA, 31 nt, Arabidopsis thaliana
  source 1..31: mol_type = other RNA
cggctggtaccatttcgatgtgtttcgattc

SEQ ID NO: 294 - RNA, 31 nt, Arabidopsis thaliana
  source 1..31: mol_type = other RNA
ctggtaccatttcgatgtgtttcgattcttc

SEQ ID NO: 295 - RNA, 31 nt, Arabidopsis thaliana
  source 1..31: mol_type = other RNA
ttgatgcttttgaattcattgtattaattcc

SEQ ID NO: 296 - RNA, 31 nt, Arabidopsis thaliana
  source 1..31: mol_type = other RNA
gcttttgaattcattgtattaattccacttc

SEQ ID NO: 297 - RNA, 31 nt, Arabidopsis thaliana
  source 1..31: mol_type = other RNA
gaattcattgtattaattccacttcctactc

SEQ ID NO: 298 - RNA, 31 nt, Arabidopsis thaliana
  source 1..31: mol_type = other RNA
actcgcggtatgctctttatgatctcggctc

SEQ ID NO: 299 - RNA, 31 nt, Arabidopsis thaliana
  source 1..31: mol_type = other RNA
ctttatgatctcggctcatgatttaattgcc

SEQ ID NO: 300 - RNA, 31 nt, Arabidopsis thaliana
  source 1..31: mol_type = other RNA
atttaattgccatgtatttagctattgagcc

SEQ ID NO: 301 - RNA, 31 nt, Arabidopsis thaliana
  source 1..31: mol_type = other RNA
aagaaagtctgaattttccacggaagccggc

SEQ ID NO: 302 - RNA, 31 nt, Arabidopsis thaliana
  source 1..31: mol_type = other RNA
gaaagtctgaattttccacggaagccggctc

SEQ ID NO: 303 - RNA, 31 nt, Arabidopsis thaliana
  source 1..31: mol_type = other RNA
ctcgaaatatttgatcttaggtgcattttcc

SEQ ID NO: 304 - RNA, 31 nt, Arabidopsis thaliana
  source 1..31: mol_type = other RNA
ttcctctggaatattattgtttggttgttcc

SEQ ID NO: 305 - RNA, 31 nt, Arabidopsis thaliana
  source 1..31: mol_type = other RNA
cacacatgttcaatctttttttagcggtttc

SEQ ID NO: 306 - RNA, 31 nt, Arabidopsis thaliana
  source 1..31: mol_type = other RNA
ccacttcgatcaattagccaagattttgacc

SEQ ID NO: 307 - RNA, 31 nt, Arabidopsis thaliana
  source 1..31: mol_type = other RNA
gatctagtggtatttttatggggattctgtc

SEQ ID NO: 308 - RNA, 31 nt, Arabidopsis thaliana
  source 1..31: mol_type = other RNA
tcctttctattgcgcctaaaatctctatttc

SEQ ID NO: 309 - RNA, 31 nt, Arabidopsis thaliana
  source 1..31: mol_type = other RNA
tctctatttctgctaatattttacgtgtttc

SEQ ID NO: 310 - RNA, 31 nt, Arabidopsis thaliana
  source 1..31: mol_type = other RNA
ccccagagatctttatcattaatgcaacctc

SEQ ID NO: 311 - RNA, 31 nt, Arabidopsis thaliana
  source 1..31: mol_type = other RNA
cccagagatctttatcattaatgcaacctcc

SEQ ID NO: 312 - RNA, 31 nt, Arabidopsis thaliana
  source 1..31: mol_type = other RNA
ccgccatggcccaaacgaaagtcaaaagacc

SEQ ID NO: 313 - RNA, 31 nt, Arabidopsis thaliana
  source 1..31: mol_type = other RNA
gcccaaacgaaagtcaaaagacctctagctc

SEQ ID NO: 314 - RNA, 31 nt, Arabidopsis thaliana
  source 1..31: mol_type = other RNA
catagttcaattggacatgtaggttatattc

SEQ ID NO: 315 - RNA, 31 nt, Arabidopsis thaliana
  source 1..31: mol_type = other RNA
gttcaattggacatgtaggttatattcgtac

SEQ ID NO: 316 - RNA, 31 nt, Arabidopsis thaliana
  source 1..31: mol_type = other RNA
tgtcggcctacgaatgtggtttcgatccttc

SEQ ID NO: 317 - RNA, 31 nt, Arabidopsis thaliana
  source 1..31: mol_type = other RNA
ttttatcttgtttcaattttatttttaatcc

SEQ ID NO: 318 - RNA, 31 nt, Arabidopsis thaliana
  source 1..31: mol_type = other RNA
tttatcttgtttcaattttatttttaatccc

SEQ ID NO: 319 - RNA, 31 nt, Arabidopsis thaliana
  source 1..31: mol_type = other RNA
gaagtaacctttttctttccttgggcagtac

SEQ ID NO: 320 - RNA, 31 nt, Arabidopsis thaliana
  source 1..31: mol_type = other RNA
taacctttttctttccttgggcagtacctcc

SEQ ID NO: 321 - RNA, 31 nt, Arabidopsis thaliana
  source 1..31: mol_type = other RNA
ctgtaatgatgtcagaatttgcaccaatttc

SEQ ID NO: 322 - RNA, 31 nt, Arabidopsis thaliana
  source 1..31: mol_type = other RNA
gatttctatatgaatggaaaaggggtgcttc

SEQ ID NO: 323 - RNA, 31 nt, Arabidopsis thaliana
  source 1..31: mol_type = other RNA
ctatatgaatggaaaaggggtgcttcggatc

SEQ ID NO: 324 - RNA, 31 nt, Arabidopsis thaliana
  source 1..31: mol_type = other RNA
ttagtgattagtctgctagtttctttgatcc

SEQ ID NO: 325 - RNA, 31 nt, Arabidopsis thaliana
  source 1..31: mol_type = other RNA
gattagtctgctagtttctttgatcctactc

SEQ ID NO: 326 - RNA, 31 nt, Arabidopsis thaliana
  source 1..31: mol_type = other RNA
gcgagaaaacaaagtgggctgtaatgatgtc

SEQ ID NO: 327 - RNA, 31 nt, Arabidopsis thaliana
  source 1..31: mol_type = other RNA
tttctttgatcctactcggtgttccttttcc

SEQ ID NO: 328 - RNA, 31 nt, Arabidopsis thaliana
  source 1..31: mol_type = other RNA
acatacagggaattggaggtagcattctacc

SEQ ID NO: 329 - RNA, 31 nt, Arabidopsis thaliana
  source 1..31: mol_type = other RNA
attctaccgatgttaagtcatggactggttc

SEQ ID NO: 330 - RNA, 31 nt, Arabidopsis thaliana
  source 1..31: mol_type = other RNA
ttcttttcattccaaattcaagaatacgacc

SEQ ID NO: 331 - RNA, 31 nt, Arabidopsis thaliana
  source 1..31: mol_type = other RNA
ccgacataagactcgacttgttagatattac

SEQ ID NO: 332 - RNA, 31 nt, Arabidopsis thaliana
  source 1..31: mol_type = other RNA
tacggaggtttagtgagcaccatgccgaatc

SEQ ID NO: 333 - RNA, 31 nt, Arabidopsis thaliana
  source 1..31: mol_type = other RNA
cggaggtttagtgagcaccatgccgaatctc

SEQ ID NO: 334 - RNA, 31 nt, Arabidopsis thaliana
  source 1..31: mol_type = other RNA
gaggtttagtgagcaccatgccgaatctctc

SEQ ID NO: 335 - RNA, 31 nt, Arabidopsis thaliana
  source 1..31: mol_type = other RNA
ccatgccgaatctctctaccattttcttttc

SEQ ID NO: 336 - RNA, 31 nt, Arabidopsis thaliana
  source 1..31: mol_type = other RNA
tcttttcttttactttggccaatatgagttc

SEQ ID NO: 337 - RNA, 31 nt, Arabidopsis thaliana
  source 1..31: mol_type = other RNA
tggtactagcagctttatcggggaatttctc

SEQ ID NO: 338 - RNA, 31 nt, Arabidopsis thaliana
  source 1..31: mol_type = other RNA
tcaagaatacgaccgatacgattaattggtc

SEQ ID NO: 339 - RNA, 31 nt, Arabidopsis thaliana
  source 1..31: mol_type = other RNA
aacctgatttcctccataaattctccgattc

SEQ ID NO: 340 - RNA, 31 nt, Arabidopsis thaliana
  source 1..31: mol_type = other RNA
aattctccgattcaaatggcagagaagtttc

SEQ ID NO: 341 - RNA, 31 nt, Arabidopsis thaliana
  source 1..31: mol_type = other RNA
ttccatatttataccttttcttgttggactc

SEQ ID NO: 342 - RNA, 31 nt, Arabidopsis thaliana
  source 1..31: mol_type = other RNA
atatttataccttttcttgttggactcgttc

SEQ ID NO: 343 - RNA, 31 nt, Arabidopsis thaliana
  source 1..31: mol_type = other RNA
tttcttgttggactcgttcggatgggtgttc

SEQ ID NO: 344 - RNA, 31 nt, Arabidopsis thaliana
  source 1..31: mol_type = other RNA
ttcggatgggtgttcaccccaaagtgttccc

SEQ ID NO: 345 - RNA, 31 nt, Arabidopsis thaliana
  source 1..31: mol_type = other RNA
gtgcctctcttattacttttttgtattctcc

SEQ ID NO: 346 - RNA, 31 nt, Arabidopsis thaliana
  source 1..31: mol_type = other RNA
ctcttattacttttttgtattctcctgttcc

SEQ ID NO: 347 - RNA, 31 nt, Arabidopsis thaliana
  source 1..31: mol_type = other RNA
cttattacttttttgtattctcctgttcctc

SEQ ID NO: 348 - RNA, 31 nt, Arabidopsis thaliana
  source 1..31: mol_type = other RNA
ggatacaattcgactcttctacggccaaatc

SEQ ID NO: 349 - RNA, 31 nt, Arabidopsis thaliana
  source 1..31: mol_type = other RNA
ctatgttagaacatttctgtgaatgctattc

SEQ ID NO: 350 - RNA, 31 nt, Arabidopsis thaliana
  source 1..31: mol_type = other RNA
tgaccacatttctgatccctatttgcatttc

SEQ ID NO: 351 - RNA, 31 nt, Arabidopsis thaliana
  source 1..31: mol_type = other RNA
gtatgagaagttatgggaaagagtatattac

SEQ ID NO: 352 - RNA, 31 nt, Arabidopsis thaliana
  source 1..31: mol_type = other RNA
gggaaagagtatattacagcatttttaattc

SEQ ID NO: 353 - RNA, 31 nt, Arabidopsis thaliana
  source 1..31: mol_type = other RNA
aattcgtgaatttctaatgatcgccgtgttc

SEQ ID NO: 354 - RNA, 31 nt, Arabidopsis thaliana
  source 1..31: mol_type = other RNA
attcgtgaatttctaatgatcgccgtgttcc

SEQ ID NO: 355 - RNA, 31 nt, Arabidopsis thaliana
  source 1..31: mol_type = other RNA
atgatcgccgtgttccgcatgctagatcttc

SEQ ID NO: 356 - RNA, 31 nt, Arabidopsis thaliana
  source 1..31: mol_type = other RNA
atgctagatcttctactattctatgtttttc

SEQ ID NO: 357 - RNA, 31 nt, Arabidopsis thaliana
  source 1..31: mol_type = other RNA
tgctagatcttctactattctatgtttttcc

SEQ ID NO: 358 - RNA, 31 nt, Arabidopsis thaliana
  source 1..31: mol_type = other RNA
tactattctatgtttttcccgaaagcgtgcc

SEQ ID NO: 359 - RNA, 31 nt, Arabidopsis thaliana
  source 1..31: mol_type = other RNA
tccaaacaggaaccaccgatttacaaatatc

SEQ ID NO: 360 - RNA, 31 nt, Arabidopsis thaliana
  source 1..31: mol_type = other RNA
agcggcgccaaatctttctatggattgcttc

SEQ ID NO: 361 - RNA, 31 nt, Arabidopsis thaliana
  source 1..31: mol_type = other RNA
ttattctgtgtcctgtgctaggaagcattac

SEQ ID NO: 362 - RNA, 31 nt, Arabidopsis thaliana
  source 1..31: mol_type = other RNA
cggcaggatccgtcatcttggcaggaattcc

SEQ ID NO: 363 - RNA, 31 nt, Arabidopsis thaliana
  source 1..31: mol_type = other RNA
ttggcaggaattcctttaaaatttggaaccc

SEQ ID NO: 364 - RNA, 31 nt, Arabidopsis thaliana
  source 1..31: mol_type = other RNA
tacccatgtttcccgaagcgacactttgttc

SEQ ID NO: 365 - RNA, 31 nt, Arabidopsis thaliana
  source 1..31: mol_type = other RNA
tcctgtgctaggaagcattactcttcttttc

SEQ ID NO: 366 - RNA, 31 nt, Arabidopsis thaliana
  source 1..31: mol_type = other RNA
ttgctataatatatacttccttgaccacttc

SEQ ID NO: 367 - RNA, 31 nt, Arabidopsis thaliana
  source 1..31: mol_type = other RNA
tgaatctggtgactattggtatgtttagtcc

SEQ ID NO: 368 - RNA, 31 nt, Arabidopsis thaliana
  source 1..31: mol_type = other RNA
aatagacgaaatattcctattatgtcaatgc

SEQ ID NO: 369 - RNA, 31 nt, Arabidopsis thaliana
  source 1..31: mol_type = other RNA
atattcctattatgtcaatgccaattgaatc

SEQ ID NO: 370 - RNA, 31 nt, Arabidopsis thaliana
  source 1..31: mol_type = other RNA
caattgaatcaatgttattagctgtgaattc

SEQ ID NO: 371 - RNA, 31 nt, Arabidopsis thaliana
  source 1..31: mol_type = other RNA
attcgaactttttggtattttccgtttcttc

SEQ ID NO: 372 - RNA, 31 nt, Arabidopsis thaliana
  source 1..31: mol_type = other RNA
cggatgatatgatgggtcaagtatttgcttc

SEQ ID NO: 373 - RNA, 31 nt, Arabidopsis thaliana
  source 1..31: mol_type = other RNA
tgatgggtcaagtatttgcttcattggttcc

SEQ ID NO: 374 - RNA, 31 nt, Arabidopsis thaliana
  source 1..31: mol_type = other RNA
tcaaatatttcacattttctatgattatttc

SEQ ID NO: 375 - RNA, 31 nt, Arabidopsis thaliana
  source 1..31: mol_type = other RNA
ttttctatgattatttctattttaggtattc

SEQ ID NO: 376 - RNA, 31 nt, Arabidopsis thaliana
  source 1..31: mol_type = other RNA
ggggaatcctccttaatagacgaaatattcc

SEQ ID NO: 377 - RNA, 31 nt, Arabidopsis thaliana
  source 1..31: mol_type = other RNA
tccttaatagacgaaatattcctattatgtc

SEQ ID NO: 378 - RNA, 31 nt, Arabidopsis thaliana
  source 1..31: mol_type = other RNA
tgggaactttgctttctggttgggaagtgtc

SEQ ID NO: 379 - RNA, 31 nt, Arabidopsis thaliana
  source 1..31: mol_type = other RNA
gtttaggtaccaatttttgggccaattcccc

SEQ ID NO: 380 - RNA, 31 nt, Arabidopsis thaliana
  source 1..31: mol_type = other RNA
tatctttgattgctttttatgaagtcgcacc

SEQ ID NO: 381 - RNA, 31 nt, Arabidopsis thaliana
  source 1..31: mol_type = other RNA
ccgaatccgagtttgctgctccaaccattac

SEQ ID NO: 382 - RNA, 31 nt, Arabidopsis thaliana
  source 1..31: mol_type = other RNA
ccaaactaatacctattctgtttagtacttc

SEQ ID NO: 383 - RNA, 31 nt, Arabidopsis thaliana
  source 1..31: mol_type = other RNA
caggtgcttttgttgcgtataatgtaaatcc

SEQ ID NO: 384 - RNA, 31 nt, Arabidopsis thaliana
  source 1..31: mol_type = other RNA
ctttcaaactagtactttttgtaatcgactc

SEQ ID NO: 385 - RNA, 31 nt, Arabidopsis thaliana
  source 1..31: mol_type = other RNA
agttttgaatgactttctagtcagatcgttc

SEQ ID NO: 386 - RNA, 31 nt, Arabidopsis thaliana
  source 1..31: mol_type = other RNA
atcattatgcctttgcaatgttacttggttc

SEQ ID NO: 387 - RNA, 31 nt, Arabidopsis thaliana
  source 1..31: mol_type = other RNA
tacttggttcaactctatttgtgaccttttc

SEQ ID NO: 388 - RNA, 31 nt, Arabidopsis thaliana
  source 1..31: mol_type = other RNA
cttggttcaactctatttgtgaccttttctc

SEQ ID NO: 389 - RNA, 31 nt, Arabidopsis thaliana
  source 1..31: mol_type = other RNA
tctatttgtgaccttttctcgtatgtgggac

SEQ ID NO: 390 - RNA, 31 nt, Arabidopsis thaliana
  source 1..31: mol_type = other RNA
actctctatcttcttgggtagataatcgatc

SEQ ID NO: 391 - RNA, 31 nt, Arabidopsis thaliana
  source 1..31: mol_type = other RNA
atgcttcttggggcttcttgttcgatagccc

SEQ ID NO: 392 - RNA, 31 nt, Arabidopsis thaliana
  source 1..31: mol_type = other RNA
cgaccgtagtgatgttaattgtggttacatc

SEQ ID NO: 393 - RNA, 31 nt, Arabidopsis thaliana
  source 1..31: mol_type = other RNA
agccctcgatttatgtgttatttatccattc

SEQ ID NO: 394 - RNA, 31 nt, Arabidopsis thaliana
  source 1..31: mol_type = other RNA
gttatttatccattcttactttttttatgcc

SEQ ID NO: 395 - RNA, 31 nt, Arabidopsis thaliana
  source 1..31: mol_type = other RNA
ttatgccaatgttggtgactggagataactc

SEQ ID NO: 396 - RNA, 31 nt, Arabidopsis thaliana
  source 1..31: mol_type = other RNA
ttacacgacttcaggcagataaagcagctac

SEQ ID NO: 397 - RNA, 31 nt, Arabidopsis thaliana
  source 1..31: mol_type = other RNA
taggtgattttggattagctcttgggatttc

SEQ ID NO: 398 - RNA, 31 nt, Arabidopsis thaliana
  source 1..31: mol_type = other RNA
gattttggattagctcttgggatttcgggtc

SEQ ID NO: 399 - RNA, 31 nt, Arabidopsis thaliana
  source 1..31: mol_type = other RNA
caaacagtagacttttcaaccatttttgctc

SEQ ID NO: 400 - RNA, 31 nt, Arabidopsis thaliana
  source 1..31: mol_type = other RNA
acttttcaaccatttttgctcgtgctagtgc

SEQ ID NO: 401 - RNA, 31 nt, Arabidopsis thaliana
  source 1..31: mol_type = other RNA
cttttcaaccatttttgctcgtgctagtgcc

SEQ ID NO: 402 - RNA, 31 nt, Arabidopsis thaliana
  source 1..31: mol_type = other RNA
gtgctagtgcccccagaaattcttggatttc

SEQ ID NO: 403 - RNA, 31 nt, Arabidopsis thaliana
  source 1..31: mol_type = other RNA
aatgccataagtcttatttgtattttacttc

SEQ ID NO: 404 - RNA, 31 nt, Arabidopsis thaliana
  source 1..31: mol_type = other RNA
gtgctgttgggaaatccgcgcagataggatc

SEQ ID NO: 405 - RNA, 31 nt, Arabidopsis thaliana
  source 1..31: mol_type = other RNA
aatccgcgcagataggatcgcatacttggtc

SEQ ID NO: 406 - RNA, 31 nt, Arabidopsis thaliana
  source 1..31: mol_type = other RNA
ctatggagggtcccactccagtatccgcttc

SEQ ID NO: 407 - RNA, 31 nt, Arabidopsis thaliana
  source 1..31: mol_type = other RNA
atagcaaggtgctcccctttatttgaatacc

SEQ ID NO: 408 - RNA, 31 nt, Arabidopsis thaliana
  source 1..31: mol_type = other RNA
acccacctacggctttgattgttattacttc

SEQ ID NO: 409 - RNA, 31 nt, Arabidopsis thaliana
  source 1..31: mol_type = other RNA
ctttgattgttattacttctgcaggagctac

SEQ ID NO: 410 - RNA, 31 nt, Arabidopsis thaliana
  source 1..31: mol_type = other RNA
cattccgttttgtttcccatcccagtctttc

SEQ ID NO: 411 - RNA, 31 nt, Arabidopsis thaliana
  source 1..31: mol_type = other RNA
taggtctcgacttcttcgctatgatcttccc

SEQ ID NO: 412 - RNA, 31 nt, Arabidopsis thaliana
  source 1..31: mol_type = other RNA
gacttcttcgctatgatcttcccagtagttc

SEQ ID NO: 413 - RNA, 31 nt, Arabidopsis thaliana
  source 1..31: mol_type = other RNA
cagtagttcatataggagctatagccgtttc

SEQ ID NO: 414 - RNA, 31 nt, Arabidopsis thaliana
  source 1..31: mol_type = other RNA
gtaccatgatactttctgttttgtcgagccc

SEQ ID NO: 415 - RNA, 31 nt, Arabidopsis thaliana
  source 1..31: mol_type = other RNA
cattgggcaatttactttatacctactattc

SEQ ID NO: 416 - RNA, 31 nt, Arabidopsis thaliana
  source 1..31: mol_type = other RNA
tatacctactattctgtctggtttttggttc

SEQ ID NO: 417 - RNA, 31 nt, Arabidopsis thaliana
  source 1..31: mol_type = other RNA
gccctgctttggtctctggtttgatggttgc

SEQ ID NO: 418 - RNA, 31 nt, Arabidopsis thaliana
  source 1..31: mol_type = other RNA
gctaaaaatccggtacattccgttttgtttc

SEQ ID NO: 419 - RNA, 31 nt, Arabidopsis thaliana
  source 1..31: mol_type = other RNA
ctaaaaatccggtacattccgttttgtttcc

SEQ ID NO: 420 - RNA, 31 nt, Arabidopsis thaliana
  source 1..31: mol_type = other RNA
atccggtacattccgttttgtttcccatccc

SEQ ID NO: 421 - RNA, 31 nt, Arabidopsis thaliana
  source 1..31: mol_type = other RNA
ctttctggtcagtaatggaagcaatcgtccc

SEQ ID NO: 422 - RNA, 31 nt, Arabidopsis thaliana
  source 1..31: mol_type = other RNA
gtcagtaatggaagcaatcgtccctaccgtc

SEQ ID NO: 423 - RNA, 31 nt, Arabidopsis thaliana
  source 1..31: mol_type = other RNA
cctaccgtcgtaaaataagagcacccggctc

SEQ ID NO: 424 - RNA, 31 nt, Arabidopsis thaliana
  source 1..31: mol_type = other RNA
accgtcgtaaaataagagcacccggctctgc

SEQ ID NO: 425 - RNA, 31 nt, Arabidopsis thaliana
  source 1..31: mol_type = other RNA
gtaaaataagagcacccggctctgcccattc

SEQ ID NO: 426 - RNA, 31 nt, Arabidopsis thaliana
  source 1..31: mol_type = other RNA
ccggctctgcccattcacaaggactcgattc

SEQ ID NO: 427 - RNA, 31 nt, Arabidopsis thaliana
  source 1..31: mol_type = other RNA
gactcgattctatgtccaaacatcacatgcc

SEQ ID NO: 428 - RNA, 31 nt, Arabidopsis thaliana
  source 1..31: mol_type = other RNA
gtccaaacatcacatgccagcagatgtggtc

SEQ ID NO: 429 - RNA, 31 nt, Arabidopsis thaliana
  source 1..31: mol_type = other RNA
tggtggaacgtgcggaaccacatattggatc

SEQ ID NO: 430 - RNA, 31 nt, Arabidopsis thaliana
  source 1..31: mol_type = other RNA
acaaaacttatcttcaagctttaccttattc

SEQ ID NO: 431 - RNA, 31 nt, Arabidopsis thaliana
  source 1..31: mol_type = other RNA
atcttcaagctttaccttattctgatcgttc

SEQ ID NO: 432 - RNA, 31 nt, Arabidopsis thaliana
  source 1..31: mol_type = other RNA
tcaagctttaccttattctgatcgttcagac

SEQ ID NO: 433 - RNA, 31 nt, Arabidopsis thaliana
  source 1..31: mol_type = other RNA
ctttcccatgacgactaggaaaaggcaaatc

SEQ ID NO: 434 - RNA, 31 nt, Arabidopsis thaliana
  source 1..31: mol_type = other RNA
tatgtttctatgatggcccaagaacacgctc

SEQ ID NO: 435 - RNA, 31 nt, Arabidopsis thaliana
  source 1..31: mol_type = other RNA
ctatgatggcccaagaacacgctcattcttc

SEQ ID NO: 436 - RNA, 31 nt, Arabidopsis thaliana
  source 1..31: mol_type = other RNA
ttacgagctcaatatatacgagtgttattcc

SEQ ID NO: 437 - RNA, 31 nt, Arabidopsis thaliana
  source 1..31: mol_type = other RNA
gagtgttattccgtgaaataactcgaatttc

SEQ ID NO: 438 - RNA, 31 nt, Arabidopsis thaliana
  source 1..31: mol_type = other RNA
tccgtgaaataactcgaatttcaaatcattc

SEQ ID NO: 439 - RNA, 31 nt, Arabidopsis thaliana
  source 1..31: mol_type = other RNA
ctaggaaaaggcaaatcaaaaattttacttc

SEQ ID NO: 440 - RNA, 31 nt, Arabidopsis thaliana
  source 1..31: mol_type = other RNA
cttctcgtatcgatgaattagaagagatgtc

SEQ ID NO: 441 - RNA, 31 nt, Arabidopsis thaliana
  source 1..31: mol_type = other RNA
tggggattcagtggtgtaatgttaagaggtc

SEQ ID NO: 442 - RNA, 31 nt, Arabidopsis thaliana
  source 1..31: mol_type = other RNA
tgttaagaggtccaggggtatgctgggattc

SEQ ID NO: 443 - RNA, 31 nt, Arabidopsis thaliana
  source 1..31: mol_type = other RNA
gattcgcgaagagcagcaccttacgatgttc

SEQ ID NO: 444 - RNA, 31 nt, Arabidopsis thaliana
  source 1..31: mol_type = other RNA
gagcagcaccttacgatgttcatgaccaatc

SEQ ID NO: 445 - RNA, 31 nt, Arabidopsis thaliana
  source 1..31: mol_type = other RNA
gcaccttacgatgttcatgaccaatcggatc

SEQ ID NO: 446 - RNA, 31 nt, Arabidopsis thaliana
  source 1..31: mol_type = other RNA
cttgacgtaccagtaggtaccagaggagatc

SEQ ID NO: 447 - RNA, 31 nt, Arabidopsis thaliana
  source 1..31: mol_type = other RNA
gacctcaacatcctgctgctcatggtgtttc

SEQ ID NO: 448 - RNA, 31 nt, Arabidopsis thaliana
  source 1..31: mol_type = other RNA
cagaggagatcgctatgatcgttactgtatc

SEQ ID NO: 449 - RNA, 31 nt, Arabidopsis thaliana
  source 1..31: mol_type = other RNA
agatcgctatgatcgttactgtatccgtatc

SEQ ID NO: 450 - RNA, 31 nt, Arabidopsis thaliana
  source 1..31: mol_type = other RNA
gatgtcgaatgaaactatccatggaatcctc

SEQ ID NO: 451 - RNA, 31 nt, Arabidopsis thaliana
  source 1..31: mol_type = other RNA
ccatttcgaactttatacagaaggtttttcc

SEQ ID NO: 452 - RNA, 31 nt, Arabidopsis thaliana
  source 1..31: mol_type = other RNA
aattgcatacctatacaagggttcaagtttc

SEQ ID NO: 453 - RNA, 31 nt, Arabidopsis thaliana
  source 1..31: mol_type = other RNA
caagtttcgatcgatatttgcggagttgatc

SEQ ID NO: 454 - RNA, 31 nt, Arabidopsis thaliana
  source 1..31: mol_type = other RNA
accagtgcagacgaagtaacacgaatatctc

SEQ ID NO: 455 - RNA, 31 nt, Arabidopsis thaliana
  source 1..31: mol_type = other RNA
ccggtagtaagtctatttccatcagccggcc

SEQ ID NO: 456 - RNA, 31 nt, Arabidopsis thaliana
  source 1..31: mol_type = other RNA
tcatcaatcatccggatctacgccgtatatc

SEQ ID NO: 457 - RNA, 31 nt, Arabidopsis thaliana
  source 1..31: mol_type = other RNA
ggtttcgagggtcatccattacgaaaagacc

SEQ ID NO: 458 - RNA, 31 nt, Arabidopsis thaliana
  source 1..31: mol_type = other RNA
aaatggaaagatcggaacatgggaatagatc

SEQ ID NO: 459 - RNA, 31 nt, Arabidopsis thaliana
  source 1..31: mol_type = other RNA
cttggaaaaaatcgtagttcagattcaagtc

SEQ ID NO: 460 - RNA, 31 nt, Arabidopsis thaliana; identical to SEQ ID NO: 459
  source 1..31: mol_type = other RNA

SEQ ID NO: 461 - RNA, 31 nt, Arabidopsis thaliana
  source 1..31: mol_type = other RNA
tggtttgacatggtttacgtgttactggttc

SEQ ID NO: 462 - RNA, 31 nt, Arabidopsis thaliana
  source 1..31: mol_type = other RNA
ggtttgacatggtttacgtgttactggttcc

SEQ ID NO: 463 - RNA, 31 nt, Arabidopsis thaliana
  source 1..31: mol_type = other RNA
cgtgttactggttcccggaagagttaatatc

SEQ ID NO: 464 - RNA, 31 nt, Arabidopsis thaliana
  source 1..31: mol_type = other RNA
gttactggttcccggaagagttaatatctcc

SEQ ID NO: 465 - RNA, 31 nt, Arabidopsis thaliana
  source 1..31: mol_type = other RNA
tcccggaagagttaatatctccattagcgtc

SEQ ID NO: 466 - RNA, 31 nt, Arabidopsis thaliana
  source 1..31: mol_type = other RNA
ggaagagttaatatctccattagcgtcaccc

SEQ ID NO: 467 - RNA, 31 nt, Arabidopsis thaliana
  source 1..31: mol_type = other RNA
cgtcaccctttcttaccctgccttttgactc

SEQ ID NO: 468 - RNA, 31 nt, Arabidopsis thaliana
  source 1..31: mol_type = other RNA
ggagaacaaaggacgaaatacaatcgattcc

SEQ ID NO: 469 - RNA, 31 nt, Arabidopsis thaliana
  source 1..31: mol_type = other RNA
gaacaaaggacgaaatacaatcgattcctcc

SEQ ID NO: 470 - RNA, 31 nt, Arabidopsis thaliana
  source 1..31: mol_type = other RNA
aatacaatcgattcctccatttaagtggttc

SEQ ID NO: 471 - RNA, 31 nt, Arabidopsis thaliana
  source 1..31: mol_type = other RNA
tacaatcgattcctccatttaagtggttctc

SEQ ID NO: 472 - RNA, 31 nt, Arabidopsis thaliana
  source 1..31: mol_type = other RNA
attcctccatttaagtggttctcgcttcttc

SEQ ID NO: 473 - RNA, 31 nt, Arabidopsis thaliana
  source 1..31: mol_type = other RNA
tctcgcttcttcttgttcctgttcctaactc

SEQ ID NO: 474 - RNA, 31 nt, Arabidopsis thaliana
  source 1..31: mol_type = other RNA
ctcgcttcttcttgttcctgttcctaactcc

SEQ ID NO: 475 - RNA, 31 nt, Arabidopsis thaliana
  source 1..31: mol_type = other RNA
cgcttcttcttgttcctgttcctaactcctc

SEQ ID NO: 476 - RNA, 31 nt, Arabidopsis thaliana
  source 1..31: mol_type = other RNA
ttcttcttgttcctgttcctaactcctcccc

SEQ ID NO: 477 - RNA, 31 nt, Arabidopsis thaliana
  source 1..31: mol_type = other RNA
cccgggtcgttcccaatgtttggcactttcc

SEQ ID NO: 478 - RNA, 31 nt, Arabidopsis thaliana
  source 1..31: mol_type = other RNA
cttcgtgggtgcaacatcaacaaattcgctc

SEQ ID NO: 479 - RNA, 31 nt, Arabidopsis thaliana
  source 1..31: mol_type = other RNA
atgatcaagttacaacctaagatctatgacc

SEQ ID NO: 480 - RNA, 31 nt, Arabidopsis thaliana
  source 1..31: mol_type = other RNA
atgaccatattatgttaactgttcgtatttc

SEQ ID NO: 481 - RNA, 31 nt, Arabidopsis thaliana
  source 1..31: mol_type = other RNA
attatgttaactgttcgtatttcgttcattc

SEQ ID NO: 482 - RNA, 31 nt, Arabidopsis thaliana
  source 1..31: mol_type = other RNA
tcgtatttcgttcattccatcggtatgctcc

SEQ ID NO: 483 - RNA, 31 nt, Arabidopsis thaliana
  source 1..31: mol_type = other RNA
cccaggtacctgtaattgtgatctgtttgcc

SEQ ID NO: 484 - RNA, 31 nt, Arabidopsis thaliana
  source 1..31: mol_type = other RNA
gtacctgtaattgtgatctgtttgccagaac

SEQ ID NO: 485 - RNA, 31 nt, Arabidopsis thaliana
  source 1..31: mol_type = other RNA
tacctgtaattgtgatctgtttgccagaacc

SEQ ID NO: 486 - RNA, 31 nt, Arabidopsis thaliana
  source 1..31: mol_type = other RNA
tatatatatcctatgaatttaatttcgcatc

SEQ ID NO: 487 - RNA, 31 nt, Arabidopsis thaliana
  source 1..31: mol_type = other RNA
acgaacaatcgtcgttttttgatggtttttc

SEQ ID NO: 488 - RNA, 31 nt, Arabidopsis thaliana
  source 1..31: mol_type = other RNA
aatcgtcgttttttgatggtttttccgcttc

SEQ ID NO: 489 - RNA, 31 nt, Arabidopsis thaliana
  source 1..31: mol_type = other RNA
tggtttttccgcttctcacagctgctctttc

SEQ ID NO: 490 - RNA, 31 nt, Arabidopsis thaliana
  source 1..31: mol_type = other RNA
ggtttttccgcttctcacagctgctctttcc

SEQ ID NO: 491 - RNA, 31 nt, Arabidopsis thaliana
  source 1..31: mol_type = other RNA
ttccacacctccggatatctggtgccaaatc

SEQ ID NO: 492 - RNA, 31 nt, Arabidopsis thaliana
  source 1..31: mol_type = other RNA
cctccggatatctggtgccaaatcgtcgccc

SEQ ID NO: 493 - RNA, 31 nt, Arabidopsis thaliana
  source 1..31: mol_type = other RNA
ggatatctggtgccaaatcgtcgcccgtttc

SEQ ID NO: 494 - RNA, 31 nt, Arabidopsis thaliana
  source 1..31: mol_type = other RNA
tgataatagagttggctatttttgtggcatc

SEQ ID NO: 495 - RNA, 31 nt, Arabidopsis thaliana
  source 1..31: mol_type = other RNA
attctaggagaagttcgaatccgttccgttc

SEQ ID NO: 496 - RNA, 31 nt, Arabidopsis thaliana
  source 1..31: mol_type = other RNA
cctttgcacgagtcagaaaccgatgtatttc

SEQ ID NO: 497 - RNA, 31 nt, Arabidopsis thaliana
  source 1..31: mol_type = other RNA
aaagctttataaagccttttgtaaagatccc

SEQ ID NO: 498 - RNA, 31 nt, Arabidopsis thaliana
  source 1..31: mol_type = other RNA
gtacaaaactgggttttggaagatatggcac

SEQ ID NO: 499 - RNA, 31 nt, Arabidopsis thaliana
  source 1..31: mol_type = other RNA
agatatggcactaaaagttgtaaagctggtc

SEQ ID NO: 500 - RNA, 31 nt, Arabidopsis thaliana
  source 1..31: mol_type = other RNA
tatttaaccataaaatcgattatgctcctgc

SEQ ID NO: 501 - RNA, 31 nt, Arabidopsis thaliana
  source 1..31: mol_type = other RNA
ataaaatcgattatgctcctgcggaagtatc

SEQ ID NO: 502 - RNA, 31 nt, Arabidopsis thaliana
  source 1..31: mol_type = other RNA
ggattgctcgtgtgtccacgggacaaatccc

SEQ ID NO: 503 - RNA, 31 nt, Arabidopsis thaliana
  source 1..31: mol_type = other RNA
gacaagccgctacattagcggctcataaacc

SEQ ID NO: 504 - RNA, 31 nt, Arabidopsis thaliana
  source 1..31: mol_type = other RNA
ccgctacattagcggctcataaaccatgttc

SEQ ID NO: 505 - RNA, 31 nt, Arabidopsis thaliana
  source 1..31: mol_type = other RNA
ctgcggaagtatctactcgttacggaatctc

SEQ ID NO: 506 - RNA, 31 nt, Arabidopsis thaliana
  source 1..31: mol_type = other RNA
atgaccctaatcgttcttctcagatcgctcc

SEQ ID NO: 507 - RNA, 31 nt, Arabidopsis thaliana
  source 1..31: mol_type = other RNA
aagttcgctccgccgcgcaagatcctcgaac

SEQ ID NO: 508 - RNA, 31 nt, Arabidopsis thaliana
  source 1..31: mol_type = other RNA
aacgcatagcattctctgggcacataggatc

SEQ ID NO: 509 - RNA, 31 nt, Arabidopsis thaliana
  source 1..31: mol_type = other RNA
aaaaatggaaaattggctatggagattccgc

SEQ ID NO: 510 - RNA, 31 nt, Arabidopsis thaliana
  source 1..31: mol_type = other RNA
ctctccgagggcatggaatgtctaatttttc

SEQ ID NO: 511 - RNA, 31 nt, Arabidopsis thaliana
  source 1..31: mol_type = other RNA
atggaatgtctaatttttcggtcagaatctc

SEQ ID NO: 512 - RNA, 31 nt, Arabidopsis thaliana
  source 1..31: mol_type = other RNA
ttccactcaattttcattacgaagatgtatc

SEQ ID NO: 513 - RNA, 31 nt, Arabidopsis thaliana
  source 1..31: mol_type = other RNA
ttcattacgaagatgtatcacgtcaagatcc

SEQ ID NO: 514 - RNA, 31 nt, Arabidopsis thaliana
  source 1..31: mol_type = other RNA
cttcggccaacacacaagatgagactttacc

SEQ ID NO: 515 - RNA, 31 nt, Arabidopsis thaliana
  source 1..31: mol_type = other RNA
gatgtatcacgtcaagatccgttgctcaaac

SEQ ID NO: 516 - RNA, 31 nt, Arabidopsis thaliana
  source 1..31: mol_type = other RNA
atgtatcacgtcaagatccgttgctcaaacc

SEQ ID NO: 517 - RNA, 31 nt, Arabidopsis thaliana
  source 1..31: mol_type = other RNA
tcacgtcaagatccgttgctcaaaccgaatc

SEQ ID NO: 518 - RNA, 31 nt, Arabidopsis thaliana
  source 1..31: mol_type = other RNA
atcacgccaacgttatggaagttcctggatc

SEQ ID NO: 519 - RNA, 31 nt, Arabidopsis thaliana
  source 1..31: mol_type = other RNA
ataaatgtccccagaaaacaggagtatgccc

SEQ ID NO: 520 - RNA, 31 nt, Arabidopsis thaliana
  source 1..31: mol_type = other RNA
cgagaacaccgaaaaaacctaattccgctcc

SEQ ID NO: 521 - RNA, 31 nt, Arabidopsis thaliana
  source 1..31: mol_type = other RNA
cggttgagcaatcgacatgatatatttgctc

SEQ ID NO: 522 - RNA, 31 nt, Arabidopsis thaliana
  source 1..31: mol_type = other RNA
ttgctcacatcccgggcgaaggtcataattc

SEQ ID NO: 523 - RNA, 31 nt, Arabidopsis thaliana
  source 1..31: mol_type = other RNA
tgttaataagaggaggtagagtgaaagattc

SEQ ID NO: 524 - RNA, 31 nt, Arabidopsis thaliana
  source 1..31: mol_type = other RNA
gtagagtgaaagattcgccaggtgtaaaatc

SEQ ID NO: 525 - RNA, 31 nt, Arabidopsis thaliana
  source 1..31: mol_type = other RNA
tagagtgaaagattcgccaggtgtaaaatcc

SEQ ID NO: 526 - RNA, 31 nt, Arabidopsis thaliana
  source 1..31: mol_type = other RNA
ggaccgtactcgagctttggataaatgtccc

SEQ ID NO: 527 - RNA, 31 nt, Arabidopsis thaliana
  source 1..31: mol_type = other RNA
caatcaattaagggtcaaatgttggaacctc

SEQ ID NO: 528 - RNA, 31 nt, Arabidopsis thaliana
  source 1..31: mol_type = other RNA
gaaagagaggaattccgtacaggtacaactc

SEQ ID NO: 529 - RNA, 31 nt, Arabidopsis thaliana
  source 1..31: mol_type = other RNA
gtatcaagatgtcaatctgagatcttatttc

SEQ ID NO: 530 - RNA, 31 nt, Arabidopsis thaliana
  source 1..31: mol_type = other RNA
gtacaggtacaactcttatttttacgaagtc

SEQ ID NO: 531 - RNA, 31 nt, Arabidopsis thaliana
  source 1..31: mol_type = other RNA
tttttacgaagtcaaaaaaatgcgatctttc

SEQ ID NO: 532 - RNA, 31 nt, Arabidopsis thaliana
  source 1..31: mol_type = other RNA
gtcagtcaaaataaaatctgtttatcaaagc

SEQ ID NO: 533 - RNA, 31 nt, Arabidopsis thaliana
  source 1..31: mol_type = other RNA
aaaataaaatctgtttatcaaagcgcttctc

SEQ ID NO: 534 - RNA, 31 nt, Arabidopsis thaliana
  source 1..31: mol_type = other RNA
aaataaaatctgtttatcaaagcgcttctcc

SEQ ID NO: 535 - RNA, 31 nt, Arabidopsis thaliana
  source 1..31: mol_type = other RNA
aggggtggagggaatccgtatatgtttttcc

SEQ ID NO: 536 - RNA, 31 nt, Arabidopsis thaliana
  source 1..31: mol_type = other RNA
aaactaaatgctataagcatagaaaaacttc

SEQ ID NO: 537 - RNA, 31 nt, Arabidopsis thaliana
  source 1..31: mol_type = other RNA
actaaatgctataagcatagaaaaacttctc

SEQ ID NO: 538 - RNA, 31 nt, Arabidopsis thaliana; identical to SEQ ID NO: 500
  source 1..31: mol_type = other RNA

SEQ ID NO: 539 - RNA, 31 nt, Arabidopsis thaliana; identical to SEQ ID NO: 501
  source 1..31: mol_type = other RNA

SEQ ID NO: 540 - RNA, 31 nt, Arabidopsis thaliana; identical to SEQ ID NO: 505
  source 1..31: mol_type = other RNA

SEQ ID NO: 541 - RNA, 31 nt, Arabidopsis thaliana
  source 1..31: mol_type = other RNA
ttcggctttcgtctcggtaggtgtattattc

SEQ ID NO: 542 - RNA, 31 nt, Arabidopsis thaliana
  source 1..31: mol_type = other RNA
accgattaccctcgataaagaagaatctttc

SEQ ID NO: 543 - RNA, 31 nt, Arabidopsis thaliana
  source 1..31: mol_type = other RNA
taccctcgataaagaagaatctttctaaatc

SEQ ID NO: 544 - RNA, 31 nt, Arabidopsis thaliana
  source 1..31: mol_type = other RNA
agcgttcctgatagaaaatgacgactccttc

SEQ ID NO: 545 - RNA, 31 nt, Arabidopsis thaliana; identical to SEQ ID NO: 459
  source 1..31: mol_type = other RNA

SEQ ID NO: 546 - RNA, 31 nt, Arabidopsis thaliana
  source 1..31: mol_type = other RNA
gttctcgtatcgctttttttgtagaaagctc

SEQ ID NO: 547 - RNA, 31 nt, Arabidopsis thaliana
  source 1..31: mol_type = other RNA
catatccctcacgacataagattaaaagatc

SEQ ID NO: 548 - RNA, 31 nt, Arabidopsis thaliana
  source 1..31: mol_type = other RNA
atatccctcacgacataagattaaaagatcc

SEQ ID NO: 549 - RNA, 31 nt, Arabidopsis thaliana
  source 1..31: mol_type = other RNA
acgacataagattaaaagatccaaaccttcc

SEQ ID NO: 550 - RNA, 31 nt, Arabidopsis thaliana
  source 1..31: mol_type = other RNA
ataagattaaaagatccaaaccttcctcttc

SEQ ID NO: 551 - RNA, 31 nt, Arabidopsis thaliana
  source 1..31: mol_type = other RNA
aagagatctattaagaagagaaagatttatc

SEQ ID NO: 552 - RNA, 31 nt, Arabidopsis thaliana
  source 1..31: mol_type = other RNA
tatatacaattacaaactacacgaaagttgc

SEQ ID NO: 553 - RNA, 31 nt, Arabidopsis thaliana
  source 1..31: mol_type = other RNA
atacaattacaaactacacgaaagttgcccc

SEQ ID NO: 554 - RNA, 31 nt, Arabidopsis thaliana
  source 1..31: mol_type = other RNA
ttacaaactacacgaaagttgcccctttttc

SEQ ID NO: 555 - RNA, 31 nt, Arabidopsis thaliana
  source 1..31: mol_type = other RNA
gaacaaaacgaacttcatatatcccttttcc

SEQ ID NO: 556 - RNA, 31 nt, Arabidopsis thaliana
  source 1..31: mol_type = other RNA
gaacttcatatatcccttttccactcaatcc

SEQ ID NO: 557 - RNA, 31 nt, Arabidopsis thaliana
  source 1..31: mol_type = other RNA
tcaatccagaaacaagatttgacgttattcc

SEQ ID NO: 558 - RNA, 31 nt, Arabidopsis thaliana
  source 1..31: mol_type = other RNA
acaagatttgacgttattccgcttcgtctcc

SEQ ID NO: 559 - RNA, 31 nt, Arabidopsis thaliana
  source 1..31: mol_type = other RNA
ttcttgaaactattcctcaagcaaggcagcc

SEQ ID NO: 560 - RNA, 31 nt, Arabidopsis thaliana
  source 1..31: mol_type = other RNA
aaataaggagatctttctataaagaaatttc

SEQ ID NO: 561 - RNA, 31 nt, Arabidopsis thaliana
  source 1..31: mol_type = other RNA
cattaagatttcaaacttgtcgtctactttc

SEQ ID NO: 562 - RNA, 31 nt, Arabidopsis thaliana
  source 1..31: mol_type = other RNA
caaacttgtcgtctactttcaggaaatgttc

SEQ ID NO: 563 - RNA, 31 nt, Arabidopsis thaliana
  source 1..31: mol_type = other RNA
tcaaaaggatcgaactacctactcattattc

SEQ ID NO: 564 - RNA, 31 nt, Arabidopsis thaliana
  source 1..31: mol_type = other RNA
gaactacctactcattattcggaggttaatc

SEQ ID NO: 565 - RNA, 31 nt, Arabidopsis thaliana
  source 1..31: mol_type = other RNA
ttaatcatagaacaccaaaagctgtggtatc

SEQ ID NO: 566 - RNA, 31 nt, Arabidopsis thaliana
  source 1..31: mol_type = other RNA
taagctataggataagcttagagaaatgttc

SEQ ID NO: 567 - DNA, 32 bp, synthetic construct
  misc_feature 1..32: note = Enko_B-F primer
  source 1..32: mol_type = other DNA
cgcgaattcagcggagagagttgcgaagcagg

SEQ ID NO: 568 - DNA, 31 bp, synthetic construct
  misc_feature 1..31: note = Enko_B-R primer
  source 1..31: mol_type = other DNA
cgcgtcgactcatcccccaaatgatagatcc

SEQ ID NO: 569 - DNA, 32 bp, synthetic construct; identical to SEQ ID NO: 567
  misc_feature 1..32: note = Kosena_B-F primer
  source 1..32: mol_type = other DNA

SEQ ID NO: 570 - DNA, 31 bp, synthetic construct; identical to SEQ ID NO: 568
  misc_feature 1..31: note = Kosena_B-R primer
  source 1..31: mol_type = other DNA

SEQ ID NO: 571 - DNA, 38 bp, synthetic construct
  misc_feature 1..38: note = Enko A-F primer
  source 1..38: mol_type = other DNA
cgcgaattcagcagggatggagagagtggcgaagcagg

SEQ ID NO: 572 - DNA, 54 bp, synthetic construct
  misc_feature 1..54: note = Enko A-R primer
  source 1..54: mol_type = other DNA
cgcgtcgacttcatcctccaactgatatcccacactcatctgcagatcctcaag

SEQ ID NO: 573 - DNA, 2064 bp, Raphanus sativus
  source 1..2064: mol_type = genomic DNA
atgttggctagggtttgtggattcaagtgttcttcttctcctgctgagtctgcggctagattgttctgtacgagatcgattcgtgatactctggccaaggcaagcggagagagttgcgaagcaggttttggaggagagagtttgaagctgcaaagtgggtttcatgaaatcaaaggtttagaggatgcgattgatttgttcagtgacatgcttcgatctcgtcctttaccttctgtggttgatttctgtaaattgatgggtgtggtggtgagaatggaacgcccggatcttgtgatttctctctatcagaagatggaaaggaaacagattcgatgtgatatatacagcttcaatattctgataaaatgtttctgcagctgctctaagctcccctttgctttgtctacatttggtaagatcaccaagcttggactccaccctgatgttgttaccttcaccaccctgctccatggattatgtgtggaagatagggtttctgaagccttggatttttttcatcaaatgtttgaaacgacatgtaggcccaatgtcgtaaccttcaccactttgatgaacggtctttgccgcgagggtagaattgtcgaagccgtagctctgcttgatcggatgatggaagatggtctccagcctacccagattacttatggaacaatcgtagatgggatgtgtaagaagggagatactgtgtctgcactgaatctgctgaggaagatggaggaggtgagccacatcatacccaatgttgtaatctatagtgcaatcattgatagcctttgtaaagacggacgtcatagcgatgcacaaaatcttttcactgaaatgcaagagaaaggaatctttcccgatttatttacctacaacagtatgatagttggtttttgtagctctggtagatggagcgacgcggagcagttgttgcaagaaatgttagaaaggaagatcagccctgatgttgtaacttataatgctttgatcaatgcatttgtcaaggaaggcaagttctttgaggctgaagaattatacgatgagatgcttccaaggggtataatccctaatacaatcacatatagttcaatgatcgatggattttgcaaacagaatcgtcttgatgctgctgagcacatgttttatttgatggctaccaagggctgctctcccaacctaatcactttcaatactctcatagacggatattgtggggctaagaggatagatgatggaatggaacttctccatgagatgactgaaacaggattagttgctgacacaactacttacaacactcttattcacgggttctatctggtgggcgatcttaatgctgctctagaccttttacaagagatgatctctagtggtttgtgccctgatatcgttacttgtgacactttgctggatggtctctgcgataatgggaaactaaaagatgcattggaaatgtttaaggttatgcagaagagtaagaaggatcttgatgctagtcaccccttcaatggtgtggaacctgatgttcaaacttacaatatattgatcagcggcttgatcaatgaagggaagtttttagaggccgaggaattatacgaggagatgccccacaggggtatagtcccagatactatcacctatagctcaatgatcgatggattatgcaagcagagccgcctagatgaggctacacaaatgtttgattcgatgggtagcaagagcttctctccaaacgtagtgacctttactacactcattaatggctactgtaaggcaggaagggttgatgatgggctggagcttttctgcgagatgggtcgaagagggatagttgctaacgcaattacttacatcactttgatttgtggttttcgtaaagtgggtaatattaatggggctctagacattttccaggagatgatttcaagtggtgtgtatcctgataccattaccatccgcaatatgctgactggtttatggagtaaagaggaactaaaaagggcagtggcaatgcttgagaaactgcagatgagtatggatctatcatttgggggatga

SEQ ID NO: 574 - DNA, 2064 bp, Raphanus sativus
  source 1..2064: mol_type = genomic DNA
atgttggctagggtttgtggattcaagtgttcttcttctcctgctgagtctgcggctagattgttctgtacgagatcgattcgtgatactctggccaaggcaagcggagagagttgcgaagcaggttttggaggagagagtttgaagctgcaaagtgggtttcatgaaatcaaaggtttagaggatgcgattgatttgttcagtgacatgcttcgatctcgtcctttaccttctgtggttgatttctgtaaattgatgggtgtggtggtgagaatggaacgcccggatcttgtgatttctctctatcagaagatggaaaggaaacagattcgatgtgatatatacagcttcaccattctgataaaatgtttctgcagctgctctaagctcccctttgctttgtctacatttggtaagatcaccaagcttggactccaccctgatgttgttaccttcaacaccctgctccacggattgtgcgtggaagatagggtttctgaagctttgaatttgtttcatcaaatgtttgaaacgacatgtaggcccaatgtcgtaaccttcaccactttgatgaacggtctttgccgcgagggtagaattgtcgaagccgtagctctgcttgatcggatgatggaagatggtctccagcctacccagattacttatggaacaatcgtagatgggatgtgtaagaagggagatactgtgtctgcattgaatcttctgaggaagatggaggaggtgagccacatcatacccaatgttgtaatctatagtgcaatcattgatagcctttgtaaagacggacgtcatagcgatgcacaaaatcttttcactgaaatgcaagagaaaggaatctttcccgatttatttacctacaacagtatgatagttggtttttgtagctctggtagatggagcgacgcggagcagttgttgcaagaaatgttagaaaggaagatcagccctgatgttgtaacttataatgctttgatcaatgcatttgtcaaggaaggcaagttctttgaggctgaagaattatacgatgagatgcttccaaggggtataatccctaatacaatcacatatagttcaatgatcgatggattttgcaaacagaatcgtcttgatgctgctgagcacatgttttatttgatggctaccaagggctgctctcccaacctaatcactttcaatactctcatagacggatattgtggggctaagaggatagatgatggaatggaacttctccatgagatgactgaaacaggattagttgctgacacaactacttacaacactcttattcacgggttctatctggtgggcgatcttaatgctgctctagaccttttacaagagatgatctctagtggtttgtgccctgatatcgttacttgtgacactttgctggatggtctctgcgataatgggaaactaaaagatgcattggaaatgtttaaggttatgcagaagagtaagaaggatcttgatgctagtcaccccttcaatggtgtggaacctgatgttcaaacttacaatatattgatcagcggcttgatcaatgaagggaagtttttagaggccgaggaattatacgaggagatgccccacaggggtatagtcccagatactatcacctatagctcaatgatcgatggattatgcaagcagagccgcctagatgaggctacacaaatgtttgattcgatgggtagcaagagcttctctccaaacgtagtgacctttactacactcattaatggctactgtaaggcaggaagggttgatgatgggctggagcttttctgcgagatgggtcgaagagggatagttgctaacgcaattacttacatcactttgatttgtggttttcgtaaagtgggtaatattaatggggctctagacattttccaggagatgatttcaagtggtgtgtatcctgataccattaccatccgcaatatgctgactggtttatggagtaaagaggaactaaaaagggcagtggcaatgcttgagaaactgcagatgagtatggatctatcatttgggggatga

SEQ ID NO: 575 - DNA, 2061 bp, Raphanus sativus
  source 1..2061: mol_type = genomic DNA
atgttggctagggtttgcagattcgagtcttcctcttcgtcttctgtgtctgcggctagatttttctgtacgggatcgattcgtcatgctctggccgagaaaagcagggatggagagagtggcgaagcaggttttagaggagagagtttgaaactgcgaagtggatcttatgaaatcaaagggttagaggatgcgattgatttgttcagtgacatgcttcgatctcgtcctttaccttctgtgattgatttcaacaagctaatgggtgcggtggtgagaatggaacgcccggatcttgtgatttctctctatcaaaagatggaaaggaaacagattcgatgtgatatatacagcttcaccattctgataaaatgtttctgcagttgctctaagctcccctttgctttgtctacatttggtaagctcaccaagcttggactccaccctgatgttgttaccttcaccaccctgctccacggattatgtcttgatcacagggtttctgaagccttggatttgtttcatcaaatttgtagaccagatgtcctaacgttcaccacgctgatgaatggtctttgccgcgagggtcgagttgtcgaagccgtagctctgcttgatcggatggtggaaaatggtctccagcctgaccagattacttacggaacatttgtagatgggatgtgtaagatgggcgacactgtgtctgcattgaatcttctgaggaagatggaggagataagccacatcaaacccaatgtggttatctatagtgccatcattgatggcctttgtaaagatggacgccatagcgattctcataatcttttcattgaaatgcaagacaagggaatctttccaaatatagttacctacaactgtatgatcggtggattttgcatctctggtagatggagtgcagcccagcggttgttgcaagaaatgttagaaaggaagatcagccctaatgttgtaacttataatgctttgatcaatgcatttgtcaaggaaggcaagttcttcgaggctgcagaattatacgatgagatgcttccaaggggtatcattcctaatacaatcacatataattcaatgatcgatgggttttgcaaacaggatcgtcttgatgctgctgaggacatgttttatttgatggctaccaagggctgctctccggacgtattcactttcactactctcatagacggatattgtggggctaagaggatagatgatggaatggaacttctccatgagatgcctagaagaggattagttgctaacacagttacttacaacactcttattcacgggttctgtctggtgggcgatcttaatgctgctctagacctttcacagcagatgatttctagtggtgtgtgccctgatatcgttacttgtaacactttgctggacggtctctgcgataatgggaaactaaaagatgcattggaaatgtttaaggctatgcagaagagtaagatggatcttgatgctagtcaccccttcaatggtgtggaacctgatgttctaacttacaatatattgatctgcggcttgatcaatgaagggaagtttttagaggccgaggaattatacgaggagatgccacacagaggtatagtcccagatactatcacctatagctcaatgatcgatggactatgcaagcagagccgcctagatgaggctacacaaatgtttgtttcgatgggtagcaagagcttctctcccaacgtagtgacatttaacacactcattaatggctactgtaaggcaggaagggttgatgatgggctggagcttttctgcgagatgggtcgaagagggatagttgctgatgcaattatttacatcactttgatttatggttttcgtaaagtgggtaatattaatggggctctagacattttccaggagatgatttcaagtggtgtgtatcctgataccattactatccgcaatatgctgactggtttttggagtaaagaggaactagaaagggcagtggcaatgcttgaggatctgcagatgagtgtggggtatcagttggaggatgaatga

SEQ ID NO: 576 - protein, 687 aa, Raphanus sativus
MLARVCGFKCSSSPAESAARLFCTRSIRDTLAKASGESCEAGFGGESLKLQSGFHEIKGLEDAIDLFSDMLRSRPLPSVVDFCKLMGVVVRMERPDLVISLYQKMERKQIRCDIYSFNILIKCFCSCSKLPFALSTFGKITKLGLHPDVVTFTTLLHGLCVEDRVSEALDFFHQMFETTCRPNVVTFTTLMNGLCREGRIVEAVALLDRMMEDGLQPTQITYGTIVDGMCKKGDTVSALNLLRKMEEVSHIIPNVVIYSAIIDSLCKDGRHSDAQNLFTEMQEKGIFPDLFTYNSMIVGFCSSGRWSDAEQLLQEMLERKISPDVVTYNALINAFVKEGKFFEAEELYDEMLPRGIIPNTITYSSMIDGFCKQNRLDAAEHMFYLMATKGCSPNLITFNTLIDGYCGAKRIDDGMELLHEMTETGLVADTTTYNTLIHGFYLVGDLNAALDLLQEMISSGLCPDIVTCDTLLDGLCDNGKLKDALEMFKVMQKSKKDLDASHPFNGVEPDVQTYNILISGLINEGKFLEAEELYEEMPHRGIVPDTITYSSMIDGLCKQSRLDEATQMFDSMGSKSFSPNVVTFTTLINGYCKAGRVDDGLELFCEMGRRGIVANAITYITLICGFRKVGNINGALDIFQEMISSGVYPDTITIRNMLTGLWSKEELKRAVAMLEKLQMSMDLSFGG

SEQ ID NO: 577 - protein, 687 aa, Raphanus sativus
MLARVCGFKCSSSPAESAARLFCTRSIRDTLAKASGESCEAGFGGESLKLQSGFHEIKGLEDAIDLFSDMLRSRPLPSVVDFCKLMGVVVRMERPDLVISLYQKMERKQIRCDIYSFTILIKCFCSCSKLPFALSTFGKITKLGLHPDVVTFNTLLHGLCVEDRVSEALNLFHQMFETTCRPNVVTFTTLMNGLCREGRIVEAVALLDRMMEDGLQPTQITYGTIVDGMCKKGDTVSALNLLRKMEEVSHIIPNVVIYSAIIDSLCKDGRHSDAQNLFTEMQEKGIFPDLFTYNSMIVGFCSSGRWSDAEQLLQEMLERKISPDVVTYNALINAFVKEGKFFEAEELYDEMLPRGIIPNTITYSSMIDGFCKQNRLDAAEHMFYLMATKGCSPNLITFNTLIDGYCGAKRIDDGMELLHEMTETGLVADTTTYNTLIHGFYLVGDLNAALDLLQEMISSGLCPDIVTCDTLLDGLCDNGKLKDALEMFKVMQKSKKDLDASHPFNGVEPDVQTYNILISGLINEGKFLEAEELYEEMPHRGIVPDTITYSSMIDGLCKQSRLDEATQMFDSMGSKSFSPNVVTFTTLINGYCKAGRVDDGLELFCEMGRRGIVANAITYITLICGFRKVGNINGALDIFQEMISSGVYPDTITIRNMLTGLWSKEELKRAVAMLEKLQMSMDLSFGG

SEQ ID NO: 578 - protein, 686 aa, Raphanus sativus
MLARVCRFESSSSSSVSAARFFCTGSIRHALAEKSRDGESGEAGFRGESLKLRSGSYEIKGLEDAIDLFSDMLRSRPLPSVIDFNKLMGAVVRMERPDLVISLYQKMERKQIRCDIYSFTILIKCFCSCSKLPFALSTFGKLTKLGLHPDVVTFTTLLHGLCLDHRVSEALDLFHQICRPDVLTFTTLMNGLCREGRVVEAVALLDRMVENGLQPDQITYGTFVDGMCKMGDTVSALNLLRKMEEISHIKPNVVIYSAIIDGLCKDGRHSDSHNLFIEMQDKGIFPNIVTYNCMIGGFCISGRWSAAQRLLQEMLERKISPNVVTYNALINAFVKEGKFFEAAELYDEMLPRGIIPNTITYNSMIDGFCKQDRLDAAEDMFYLMATKGCSPDVFTFTTLIDGYCGAKRIDDGMELLHEMPRRGLVANTVTYNTLIHGFCLVGDLNAALDLSQQMISSGVCPDIVTCNTLLDGLCDNGKLKDALEMFKAMQKSKMDLDASHPFNGVEPDVLTYNILICGLINEGKFLEAEELYEEMPHRGIVPDTITYSSMIDGLCKQSRLDEATQMFVSMGSKSFSPNVVTFNTLINGYCKAGRVDDGLELFCEMGRRGIVADAIIYITLIYGFRKVGNINGALDIFQEMISSGVYPDTITIRNMLTGFWSKEELERAVAMLEDLQMSVGYQLEDE

SEQ ID NO: 579 - DNA, 41 bp, synthetic construct
  misc_feature 1..41: note = A-F primer primer
  source 1..41: mol_type = other DNA
aatacgactcactatagaagaagaagctgctttgtggagtg

SEQ ID NO: 580 - DNA, 24 bp, synthetic construct
  misc_feature 1..24: note = A-R primer primer
  source 1..24: mol_type = other DNA
tgcatttatatgctgaagaaaagc

SEQ ID NO: 581 - DNA, 48 bp, synthetic construct
  misc_feature 1..48: note = B-F primer primer
  source 1..48: mol_type = other DNA
tagataatacgactcactatagggatgattacctttttcgaaaaattg

SEQ ID NO: 582 - DNA, 38 bp, synthetic construct
  misc_feature 1..38: note = B-R primer primer
  source 1..38: mol_type = other DNA
aaaccagcttagccaaatatgggccattagcaaataag

SEQ ID NO: 583 - DNA, 42 bp, synthetic construct
  misc_feature 1..42: note = C-F primer primer
  source 1..42: mol_type = other DNA
aatacgactcactatagtatttggctaagctggttttctaac

SEQ ID NO: 584 - DNA, 27 bp, synthetic construct
  misc_feature 1..27: note = C-R primer primer
  source 1..27: mol_type = other DNA
gacacctagccacccatactgaaattc

SEQ ID NO: 585 - protein, 667 aa, Raphanus sativus
MLARVCRFESSSSVPAARLFCTRSIRHTLAKKSSGKAGGFGGERLKLQSGFHEIKGLDDAIDLFGYMVRSRPLPCVIDFCKLLGVVVRMERPDVVISLHRKMEMRRIPCNIYSFTILIKCFCSCSKLPFALSTFGKITKLGFHPSLVTFSTLLHGLCVEDRVSEALHFFHQICKPNVIAFTTLMNGLCREGRVVEAVALLDRMVEDGLQPNQITYGTIVDGMCKMGDTVSALNLLRKMEEVSRIKPNVVIYSAIIDGLWKDGRQTDAQNLFSEMQEKGISPNLFTYNCMINGFCSSGRWSEAQRLLREMFERKMSPDVVTFSVLINALVKEGKFFEAEELYNEMLPRGIIPNTITYNSMIDGFSKQNRLDAAERMFYLMATKGCSPDVITFSILIDGYCGAKRVDDGMKLLHEMSRRGLVANTITYTTLIHGFCQLGNLNAALDLLQEMISSGVCPNVVTCNTLLDGLCNNGKLKDALEMFKVMQKSKMDLDASHPFNDVEPDVQTYNILICGLINEGKFSEAEELYEEMPHRGLVPDTITYNSVIDGLCKQSRLDEATQMFDSMGSKGFSPDVVTFTTLINGYCKVGRVGDGLEVFCEMGRRGIVANAITYRTLIHGFCQVGNINGALDIFQEMISSGVYPDTITIRNMLTGLWSKEELKRAVQCL

SEQ ID NO: 586 - protein, 683 aa, Raphanus sativus
MLARVCRFESSSSSSVSAARFFCTGSIRHALAEKSRDGESGEAGFRGESLKLRSGSYEIKGLEDAIDLFSDMLRSRPLPSVIDFNKLMGAVVRMERPDLVISLYQKMERKQIRCDIYSFTILIKCFCSCSKLPFALSTFGKLTKLGLHPDVVTFTTLLHGLCLDHRVSEALDLFHQICRPDVLTFTTLMNGLCREGRVVEAVALLDRMVENGLQPDQITYGTFVDGMCKMGDTVSALNLLRKMEEISHIKPNVVIYSAIIDGLCKDGRHSDSHNLFIEMQDKGIFPNIVTYNCMIGGFCISGRWSAAQRLLQEMLERKISPNVVTYNALINAFVKEGKFFEAAELYDEMLPRGIIPNTITYNSMIDGFCKQDRLDAAEDMFYLMATKGCSPDVFTFTTLIDGYCGAKRIDDGMELLHEMPRRGLVANTVTYNTLIHGFCLVGDLNAALDLSQQMISSGVCPDIVTCNTLLDGLCDNGKLKDALEMFKAMQKSKMDLDASHPFNGVEPDVLTYNILICGLINEGKFLEAEELYEEMPHRGIVPDTITYSSMIDGLCKQSRLDEATQMFVSMGSKSFSPNVVTFNTLINGYCKAGRVDDGLELFCEMGRRGIVADAIIYITLIYGFRKVGNINGALDIFQEMISSGVYPDTITIRNMLTGFWSKEELERAVAMLEDLQRYQLEDE

SEQ ID NO: 587 - protein, 687 aa, Raphanus sativus
MLARVCGFKCSSSPAESAARLFCTRSIRDTLAKASGESCEAGFGGESLKLQSGFHEIKGLEDAIDLFSDMLRSRPLPSVVDFCKLMGVVVRMKRPDVVISLHKKVEMRRIPCDAYSFNILIKCFCSCSKLPFALSTFGKITKLGLHPDVVTFTTLLHGLCVEDRVSEALNLFHQMFETTCRPNVVTFTTLMNGLCREGRIVEAVALLDRMMEDGLQPTQITYGTIVDGMCKKGDTVSALNLLRKMEEVSHIIPNVVIYSAIIDSLCKDGRHSDAQNLFTEMQEKGIFPDLFTYNSMIVGFCSSGRWSDAEQLLQEMLERKISPDVVTYNALINAFVKEGKFFEAEELYDEMLPRGIIPNTITYSSMIDGFCKQNRLDAAENMFYLMATKGCSPNLITFNTLIDGYCGAKRIDDGMELLHEMTETGLVADTTTYNTLIHGFCLVGDLNAALDLLQEMISSGLCPDIVTCDTLLDGLCDNGKLKDALEMFKVMQKSKKDLDASHPFNGVEPDVQTYNILISGLINEGKFLEAEELYEEMPHRGIVPDTITYSSMIDGLCKQSRLDEATQMFDSMGSKSFSPNVVTFTTLINGYCKAGRVDDGLELFCEMGRRGIVANAITYITLICGFRKVGNINGALDIFQEMISSGVYPDTITIRNMLTGLWSKEELKRAVAMLEKLQMSMDLSFGG

SEQ ID NO: 588 - protein, 690 aa, Raphanus sativus
MLARVCRFESSSSSSVSAARMFCTGSIRHALAKKGRDGESGEAGFGGESLKLRSGFHEIKGLEDAIDLFSDMLRSRPLPSVIDFNKLMGVVVRMERPDLVISLYQKMERKQIPCDVYSFNILIKCFCSCSKLPFALSTFGKITKLGLHPDVATFNTLLHGLCLDKRVSEALDLFHQMFETTCRPNIITFTTLMNGLCYEGRVVEAVALLDRMLEDGLQPDQITYGTIVDGMCKMGDTVSALNLLRKMEELSHIKPNVVIYSAIIDGLWKDGRHTDAQNLFSEMQEKGIFPNLFTYTCMINGFCDSGRWSEAQQLLQEMLVRKISPNVVTYSALINAFVKEGKFFEAEELYDEMLPRGIIPSTVTYSSMIDGFCKQNRLDAAEHMFYLMPTKGCSPNLITFNTLIAGYCRAKRVDDGMELLHEMTETGLVADTTTYNTLIHGFCLVDDLNAALDLSQQMISSGVCPDIVTCNTLLDGLCDNGKLKDALEMFKAMQKSKMDFDASHPFNDVEPDVLTYNILISGLINDGKFLGAEELYEEMPHRGIVPDTFTYNSMICGLCKQNRLDEAKQMSDSMGSKSFSPNVVTFNTLINGYCKAGRVDDGLELFCGMGQRGIVANAITYITLIHGFRKVDNINGALDIFQEMISSGVYPDTITIRNMLTGLWSKEELKRAVAMLEDLQMSVGYQLEDE

SEQ ID NO: 589 - protein, 690 aa, Raphanus sativus
MLARVCRFESSSSSSVSAARMFCTGSIRHALAKKGRDGESGEAGFGGESLKLRSGFHEIKGLEDAIDLFSDMLRSRPLPSVIDFNKLMGVVVRMERPDLVISLYQKMERKQIPCDVYSFNILIKCFCSCSKLPFALSTFGKITKLGLHPDVATFNTLLHGLCLDKRVSEALDLFHQMFETTCRPNIITFTTLMNGLCYEGRVVEAVALLDRMLEDGLQPDQITYGTIVDGMCKMGDTVSALNLLRKMEELSHIKPNVVIYSAIIDGLWKDGRHTDAQNLFSEMQEKGIFPNLFTYTCMINGFCDSGRWSEAQQLLQEMLVRKISPNVVTYSALINAFVKEGKFFEAEELYDEMLPRGIIPSTVTYSSMIDGFCKQNRLDAAEHMFYLMPTKGCSPNLITFNTLIAGYCRAKRVDDGMELLHEMTETGLVADTTTYNTLIHGFCLVDDLNAALDLSQQMISSGVCPDIVTCNTLLDGLCDNGKLKDALEMFKAMQKSKMDFDASHPFNDVEPDVLTYNILISGLINDGKFLGAEELYEEMPHRGIVPDTVTYNSVINGLCKQSRLNEAKQMSDSMGSRSFSPNVVTFNTLINGYCKAGRVDDGLELFCEMGRRGVVANAITYITLIHGFSKVGNINGALDIFQEMMASGVYPDTITIRNMLTGLWSKEEVKRAVAMLEDLQMSVGYQLEDE

SEQ ID NO: 590 - protein, 683 aa, Raphanus sativus
MLARVYRSGSSSSPAVSAARLFCTRSIRHALAKKSRDGESGFGGESLKLRSGFHEIKGLEDAIDLFGDMVRSRPLPSVIDFCKLMGVVVRMGRLDVVISLHRKMEMGRVPCNAYSFTILMKCFCSCSKLPFALSTFGKITKLGLHPDVVTFNTLLHGLCVEDRVSEALYLFHQMCKPNVVTFTTLMKGLCREGRVVEAVALLDRMVEDGLQPNQITYGTIVDGMCKMGDSVSALDLLRKMEEVSHIKPDVVIYSAIIDGLWKDGRHTDAQNLFSEMQDKRIFPDLFTYSCMIDGFCSSGRWSEAQQLLQEMLERKISPDVVTYNALINAFVKEGKFFEAEELYDEMLPRGIIPNTITYSSMIDGFCKQNRLDAAEHMFYLMATKGCSPDVFTFNTLIDGYCGAKRIDDGMELLHEMTEAGLVANTVTYTTLIHGFCQVGDLNSAQDLLQEMISSGVCPNVVTCNTLLDGLCDNGKLKDALKMFKAMQKSKKNFDASHPFNGVEPDVLTYNILICGLINEGKFIEAEELYEEMPHRGIVPDTITYSSMIDGLCKQSRLDEATQMSDSMGSKSFSPNVVTFNTLINGYCKAGRVDDGLELFCEMGRRGIVANAITYITLIYGSRKVGNINGALDIFQEMISSGVYPDTITIRNMLTGLWSKEELERAVAMLEVLQMSVGYQLEDE

SEQ ID NO: 591 - protein, 686 aa, Raphanus sativus
MLARVCRFESSSSSSVSAARLLCTRSIHLPLAEKSRDGENGEAGSGGESLKLQSGSYEIKGLEDAIDLFSDMLRSRPLPSVIDFNKLMGAVVRMERPDLVISLYQKMERKQIRCDIYSFTILIKCFCSCSKLPVALSTFGKLTKLGLHPDVVTFTTLLHGLCVEDRVSEALDLFHQMCKPNVVTFNTLMNGLCREGRVVEAVALLDQMVENGLQPDQITYGTIVDGMCKMGDTVSALNLLRKMEEVSHIIPNVVIYSAIIDGLCKDGRHSDAHNLFIEMQDKGIFPNIVTYNCMIGGFCISGRWSAAQRLLQEMLVRKISPNVVTYNALINAFVKEGKFFEAEELYDEMLPRGIIPNTITYNSMIDGFCKQNRLDAAEHMFYVMATKGCSPDVFTFNTLIDGYCGAKRIDDGMELLHEMTETGLVADTTTYNTLIHGFCLVGDLNAALDLLQEMVSSGVCPDIVTCNTLLDGLCDNGKLKDALEMFKAMQKSKMYIDASHPFNGVEPDVLTYNILICGLINEGKFLEAEELYEEMPHRGIVPDTITYSSMIDGLCKQSRLDEATQMFVSMGSKSFSPNVVTFNTLINGYCKAGRVDDGLELFCEMGRRGIVADAITYITLIYGFRKVGNINGALDIFQEMMASGVYPDTITIRNMLTGLWSKEELKRAVAMLEDLQMSVGYQLEDE